(19) United States
(12) Patent Application Publication
(10) Pub. No.: US 2024/0180467 A1
(43) Pub. Date: Jun. 6, 2024

(54) Measuring urine production and other urine-related parameters

(71) Applicant: FIZE MEDICAL LTD., Modiin (IL)

(72) Inventors: Noam Levine, Modiin (IL); Yuval Kolodny, Haifa (IL); Baruch Yehuda Hermann, Zait Raanan – Talmon (IL)

(21) Appl. No.: 18/550,292

(22) PCT Filed: Apr. 14, 2022
(86) PCT No.: PCT/IB2022/053520

§ 371 Date: Sep. 13, 2023

Related U.S. Application Data
(60) Provisional application No. 63/201,125, filed on Apr. 14, 2021.

(51) Int. Cl. A61B 5/20; A61B 5/00; A61M 25/00

(52) U.S. Cl. CPC A61B 5/208; A61B 5/6852; A61M 25/0017

(57) ABSTRACT

An apparatus (129) for use with a conduit (371) that is configured to carry urine downstream from a bladder (122) of a subject includes one or more force-applying elements (22, 350, 352) configured to reversibly couple to the conduit (371) and apply force to the conduit (371) when coupled to the conduit (371). The apparatus (129) further includes a controller (125) configured to control the force-applying elements (22, 350, 352) such that the force-applying elements (22, 350, 352) apply the force to the conduit (371), thereby forcing the urine from the conduit (371) in a downstream direction, and to calculate a volume of the urine that was forced, based on the controlling of the force-applying elements (22, 350, 352). Other embodiments are also described.

### CROSS-REFERENCE TO RELATED APPLICATIONS

The present application claims the benefit of U.S. Provisional Application 63/201,125, filed Apr. 14, 2021, whose disclosure is incorporated herein by reference.

### FIELD OF THE INVENTION

The present invention relates generally to the field of medical devices, and particularly to systems and methods for facilitating the diagnosis and/or treatment of a subject connected to a urinary catheter.

### BACKGROUND

Co-assigned U.S. Pat. No. 9,752,914 to Levine describes a method, device, and system for determining a flow rate of an excretion stream within an excretion collection assembly. In some embodiments, one of the constituent elements of the collection assembly includes a sensing module including an electrical and/or electromechanical component.

Co-assigned U.S. Pat. No. 10,240,590 to Levine describes a fluid flow meter comprising a fluid pump configured to displace fluid with pumping strokes of one or more pumping stroke types, wherein each of the one or more stroke types displaces a known volume of fluid. The fluid flow meter further comprises a sensor functionally associated with a fluid reservoir and adapted to generate a signal indicative of a fluid pumping condition within the fluid reservoir, which fluid reservoir is integral or functionally associated with the pump, and circuitry configured to trigger one stroke or a sequence of strokes of the pump in response to a signal from the sensor.

International Patent Application Publication WO/2019/106674 describes a dual active valve positive displacement pump comprising a housing holding the pump's components, and a piston with an internal cavity divided into two fluidly-isolated volumes by a freely-moving diaphragm, one of the two volumes being fluidly connected with a volume between the piston and the housing that contains driver pressure from a pressure source. The piston is reciprocally movable inside the housing under positive or negative driver pressure. An active inlet valve operable by driver pressure actuates when the driver pressure is more than the maximum pressure at the pump inlet port. An active outlet valve operable by driver pressure actuates when the driver pressure is less than the minimum pressure at the pump outlet port. The diaphragm separates pumped fluid from operational fluid used to move the diaphragm inside the piston cavity and transmits pressure at the inlet port when the inlet valve is open, and at the outlet port when the outlet valve is open.

International Patent Application Publication WO/2019/106675 describes a device for measuring a rate of production of urine in a subject comprising a catheter, a pressure transducer, and a means for measuring the amount of urine produced. Urine flow from the bladder is prevented until a predetermined pressure is reached in the bladder. Urine flow is then allowed, and the bladder pressure and the volume of urine exiting the bladder are measured. When the bladder pressure reaches a second, lower predetermined pressure, urine flow is again prevented. From the measured pressure during urine flow and from the volume of urine exiting the bladder, the intra-abdominal pressure and the urine production rate can be determined.

### SUMMARY OF THE INVENTION

There is provided, in accordance with some embodiments of the present invention, an apparatus for use with a conduit that is configured to carry urine downstream from a bladder of a subject. The apparatus includes one or more force-applying elements configured to reversibly couple to the conduit and to apply force to the conduit when coupled to the conduit. The apparatus further includes a controller configured to control the force-applying elements such that the force-applying elements apply the force to the conduit, thereby forcing the urine from the conduit in a downstream direction, and to calculate a volume of the urine that was forced, based on the controlling of the force-applying elements.

In some embodiments, the apparatus further includes the conduit.

In some embodiments, the force-applying elements include an actuator configured to reversibly couple to the conduit, and to apply the force to the conduit, via a fluid in a fluid-filled tube.

In some embodiments, the apparatus further includes a pressure sensor configured to:
couple to the fluid-filled tube so as to sense a fluid pressure of the fluid, and
communicate, to the controller, a signal indicating the fluid pressure,
and the controller is configured to control the force-applying elements responsively to the signal.

In some embodiments, the conduit includes a chamber including a moveable wall, and the force-applying elements are configured to apply the force to the moveable wall.

In some embodiments, the apparatus further includes a case coupled to the force-applying elements, and the force-applying elements are configured to reversibly couple to the conduit by virtue of the case reversibly coupling to the conduit.

In some embodiments, the conduit is at least partly contained in a cartridge, the case is shaped to define a slot, and the case is configured to reversibly couple to the conduit via insertion of the cartridge into the slot.

In some embodiments, the conduit is coupled to one or more latches, and the case is configured to reversibly couple to the conduit by virtue of the latches latching onto the case.

In some embodiments, the case includes one or more latches configured to latch onto a housing of the conduit, thereby reversibly coupling the case to the conduit.

In some embodiments, the case includes:
an electrical interface connected to the controller, and configured to couple to a cable such that the controller is powered via the cable; and
a communication interface connected to the controller and configured to couple to the cable,
and the controller is configured to communicate the calculated volume, or a parameter derived therefrom, via the communication interface and the cable.

In some embodiments, the force-applying elements include:
a pressing element; and
an actuator, configured to apply the force to the conduit by causing the pressing element to press against the conduit,
and the controller is configured to control the actuator.

In some embodiments, the conduit includes a tube, and the pressing element is configured to press against the tube.

In some embodiments, the pressing element includes a rotor configured to rotate while pressing against the tube.

In some embodiments, the actuator is further configured to measure a reciprocal force exerted by the conduit on the pressing element, and the controller is configured to control the actuator responsively to the reciprocal force.

In some embodiments, the actuator includes an encoder configured to detect a position of the pressing element, and the controller is configured to control the actuator responsively to the position.

In some embodiments, the apparatus further includes a sensor configured to communicate, to the controller, a signal that varies as a function of an amount of the urine in the conduit or in the bladder, and the controller is configured to control the force-applying elements responsively to the signal.

In some embodiments, the sensor includes a pressure sensor configured to couple to the conduit so as to sense a pressure in the conduit, and the signal indicates the pressure.

In some embodiments,
the conduit includes an expandable portion configured to expand as the urine flows into the expandable portion,
the sensor is configured to sense a degree of expansion of the expandable portion, and
the signal indicates the degree of expansion.

In some embodiments, the expandable portion includes a reservoir disposed upstream from a portion of the conduit to which the force is applied.

In some embodiments, the expandable portion includes a moveable wall and is configured to expand via movement of the moveable wall, and the force-applying elements are configured to apply the force to the moveable wall.

In some embodiments, the sensor includes a pressure sensor configured to sense a pressure that varies with the degree of expansion.

In some embodiments, the sensor includes an optical sensor configured to sense the degree of expansion by emitting light at the expandable portion.

In some embodiments, the conduit is coupled to a first electrical interface configured to connect to the sensor, and the force-applying elements are coupled to a second electrical interface connected to the controller and configured to contact the first electrical interface, when the force-applying elements are coupled to the conduit, such that the sensor communicates the signal to the controller via the first electrical interface and second electrical interface.

In some embodiments, the apparatus further includes a pressure-conveying tube configured to couple to the conduit and to contain a fluid such that a fluid pressure of the fluid varies with an internal pressure in the conduit,
the sensor includes a pressure sensor configured to couple to the pressure-conveying tube so as to sense the fluid pressure, and
the signal indicates the fluid pressure.

In some embodiments, the apparatus further includes an optical sensor configured to sense a visual parameter of the urine and to communicate, to the controller, a signal indicating the visual parameter.

There is further provided, in accordance with some embodiments of the present invention, a method for use with a conduit that is configured to carry urine downstream from a bladder of a subject. The method includes controlling one or more force-applying elements, which are reversibly coupled to the conduit, such that the force-applying elements apply force to the conduit, thereby forcing the urine from the conduit in a downstream direction, and calculating a volume of the urine that was forced, based on the controlling of the force-applying elements.

There is further provided, in accordance with some embodiments of the present invention, an apparatus for use with one or more force-applying elements. The apparatus includes at least one tube, configured to carry urine that flows downstream from a bladder of a subject via a urinary catheter that catheterizes the subject. The apparatus further includes a conduit section configured to couple to the tube in fluid communication with the tube and to reversibly couple to the force-applying elements so as to facilitate the force-applying elements applying force to the conduit section, thereby forcing the urine from the conduit section in a downstream direction.

In some embodiments, the at least one tube includes an upstream tube connected to an upstream end of the conduit section, and the apparatus further includes:
a bypass tube connected to the upstream tube; and
a valve configured to:
   prevent a flow of the urine through the bypass tube when a pressure within the bypass tube is less than a predetermined threshold, and
   allow the flow when the pressure exceeds the threshold, such that the urine bypasses the conduit section.

In some embodiments,
the at least one tube further includes a downstream tube connected to a downstream end of the conduit section,
the apparatus further includes a connector configured to connect the downstream tube to a urine-collection bag, and
the bypass tube is connected to the connector, such that the bypass tube passes between the upstream tube and the connector.

In some embodiments, the valve is integrated into the connector.

In some embodiments, the force-applying elements are coupled to a case, and the conduit section is configured to reversibly couple to the force-applying elements by reversibly coupling to the case.

In some embodiments, the apparatus further includes a cartridge containing the conduit section.

In some embodiments,
the case is shaped to define a slot, and
the conduit section is configured to reversibly couple to the case via insertion of the cartridge into the slot.

In some embodiments, the apparatus further includes one or more latches coupled to the conduit section and configured to latch onto the case, thereby reversibly coupling the conduit section to the case.

In some embodiments, the apparatus further includes a housing that houses the conduit section,
the case includes one or more latches configured to latch onto the housing, and
the conduit section is configured to reversibly couple to the case by virtue of the latches latching onto the housing.

In some embodiments, the force-applying elements include a pressing element configured to apply the force to the conduit section by pressing against the conduit section.

In some embodiments, the conduit section includes a peristaltic pump tube, and the conduit section is configured to reversibly couple to the pressing element so as to facilitate the pressing element pressing against the peristaltic pump tube.

In some embodiments, the conduit section includes a chamber including a moveable wall, and the conduit section is configured to reversibly couple to the force-applying elements so as to facilitate the force-applying elements applying the force to the moveable wall.

In some embodiments, the apparatus further includes a reservoir disposed upstream from the conduit section.

In some embodiments, the reservoir is configured to expand as the urine flows into the reservoir.

In some embodiments, the apparatus further includes a sensor configured to communicate a signal that varies as a function of an amount of the urine in the reservoir.

In some embodiments, the apparatus further includes a sensor configured to communicate a signal that varies as a function of an amount of the urine in the conduit section.

In some embodiments, the apparatus further includes a pressure sensor configured to sense an outlet pressure at an outlet of the urinary catheter and to communicate a signal indicating the outlet pressure.

In some embodiments, the apparatus further includes a connection port coupled to the tube or to the conduit section and configured to couple to a pressure sensor such that the pressure sensor senses an internal pressure in the tube or in the conduit section.

In some embodiments, the apparatus further includes a connection port coupled to the tube or to the conduit section and configured to couple to a pressure-conveying tube containing a fluid such that a fluid pressure in the pressure-conveying tube varies in response to an internal pressure in the tube or in the conduit section.

In some embodiments, the apparatus further includes the pressure-conveying tube.

In some embodiments, the apparatus further includes a first electrical interface coupled to the conduit and configured to connect to a sensor, and the force-applying elements are coupled to a second electrical interface connected to a controller and configured to contact the first electrical interface, when the conduit section is coupled to the force-applying elements, such that the sensor communicates a signal to the controller via the first electrical interface and second electrical interface.

There is further provided, in accordance with some embodiments of the present invention, a system including a pump and a controller. The controller is configured to pump urine from a bladder of a subject, by controlling the pump, and to generate an alert indicating a current or likely upcoming disruption to the pumping.

In some embodiments, the disruption includes an inhibited flow of the urine downstream from the pump.

In some embodiments, the controller is configured to generate the alert in response to an increased amount of power consumed by the pump.

In some embodiments,
the controller is further configured to calculate an amount of the urine that was pumped by the pump,
the controller is configured to pump the urine into a collection bag, and
the controller is configured to generate the alert in response to a difference between a maximum capacity of the collection bag and the amount of the urine that was pumped being less than a predefined threshold.

In some embodiments, the disruption includes an inhibited flow of the urine upstream from the pump.

In some embodiments, the controller is configured to pump the urine through a conduit connected to a urinary catheter that catheterizes the subject, and the inhibited flow is due to a blockage in the conduit upstream from the pump or in the urinary catheter.

In some embodiments,
the conduit includes a reservoir,
the blockage is downstream from the reservoir, and
the controller is configured to generate the alert in response to a signal indicating that an amount of the urine that flowed from the reservoir is less than a pumping volume of the pump.

In some embodiments,
a pressure sensor is coupled to the conduit so as to sense a pressure,
the blockage is downstream from the pressure sensor, and
the controller is configured to generate the alert in response to a change in the pressure.

In some embodiments,
the conduit includes a reservoir,
the blockage is upstream from the reservoir, and
the controller is configured to generate the alert in response to a signal indicating that an increase in an amount of the urine in the reservoir is less than a predefined threshold.

In some embodiments,
a pressure sensor is coupled to the conduit so as to sense a pressure,
the blockage is upstream from the pressure sensor, and
the controller is configured to generate the alert in response to an increase in the pressure being less than a predefined threshold.

There is further provided, in accordance with some embodiments of the present invention, a method, including pumping urine from a bladder of a subject, by controlling a pump, and generating an alert indicating a current or likely upcoming disruption to the pumping.

There is further provided, in accordance with some embodiments of the present invention, a system including a pump and a controller. The controller is configured to continually receive a signal that varies as a function of an amount of urine in a bladder of a subject or in a conduit connected to a urinary catheter that catheterizes the subject, and, in response to the signal, using the pump, pump the urine through the conduit so as to keep the amount of urine in the bladder within a range of 20 ml.

In some embodiments, the controller is configured to pump the urine through the conduit such that the amount of urine in the bladder remains less than 20 ml.

In some embodiments, the controller is configured to pump the urine through the conduit so as to keep a pressure in the conduit less than an atmospheric pressure.

In some embodiments, the signal indicates a pressure within the conduit.

In some embodiments, the signal indicates a fluid pressure of a fluid contained within a tube coupled to the conduit.

In some embodiments, the conduit includes an expandable portion configured to expand as the urine flows into the expandable portion, and the signal indicates a degree of expansion of the expandable portion.

In some embodiments, the controller is configured to activate the pump in response to the signal crossing a predefined threshold.

In some embodiments, the controller is configured to activate the pump in response to the signal crossing the predefined threshold in a first direction, and the controller is further configured to stop the pump in response to the signal crossing the predefined threshold in a second direction opposite from the first direction.

In some embodiments,
the predefined threshold is a first predefined threshold, and
the controller is configured to stop the pump in response to the signal crossing a second predefined threshold in the second direction after crossing the first predefined threshold in the second direction.

In some embodiments, the controller is further configured to stop the pump in response to the pump having pumped a predefined volume of the urine.

There is further provided, in accordance with some embodiments of the present invention, a method, including continually receiving a signal that varies as a function of an amount of urine in a bladder of a subject or in a conduit connected to a urinary catheter that catheterizes the subject, and, in response to the signal, using a pump, pumping the urine through the conduit so as to keep the amount of urine in the bladder within a range of 20 ml.

There is further provided, in accordance with some embodiments of the present invention, a system including a display and a processor. The processor is configured to obtain a noisy signal representing a rate of urine production by kidneys of a subject as a function of time, to filter noise from the noisy signal so as to obtain a clean signal, to compute a representative rate of change in the clean signal over at least 12 hours, and to display an output, indicating the representative rate of change, on the display.

In some embodiments, the output includes a graphical output.

In some embodiments, the processor is further configured to generate an alert in response to a magnitude of the representative rate of change exceeding a predefined threshold.

There is further provided, in accordance with some embodiments of the present invention, a method including obtaining a noisy signal representing a rate of urine production by kidneys of a subject as a function of time, filtering noise from the noisy signal so as to obtain a clean signal, computing a representative rate of change in the clean signal over at least 12 hours, and generating an output indicating the representative rate of change.

There is further provided, in accordance with some embodiments of the present invention, a system including a conduit, configured to connect to a urinary catheter that catheterizes a subject, and a controller. The controller is configured to ascertain that urine has at least partly ceased to flow downstream from a bladder of the subject through the conduit, and in response to the ascertaining, increase a pressure in the conduit.

In some embodiments, the controller is configured to increase the pressure by causing the urine to flow upstream, toward the bladder.

In some embodiments, the controller is configured to increase the pressure by pressing the conduit.

In some embodiments, the system further includes a plunger, and the controller is configured to press the conduit using the plunger.

In some embodiments, the controller is further configured to:
ascertain that the urine has resumed flowing from the bladder, and
in response to ascertaining that the urine has resumed flowing from the bladder, stop increasing the pressure.

In some embodiments, the system further includes a pump, and the controller is further configured to:
using the pump, cause the urine to flow downstream by pumping the urine downstream, and
in response to the ascertaining, stop pumping the urine downstream.

In some embodiments, the controller is configured to increase the pressure by operating the pump in an upstream pumping direction.

In some embodiments, the controller is further configured to resume pumping the urine downstream in response to ascertaining that the urine has resumed flowing from the bladder.

There is further provided, in accordance with some embodiments of the present invention, a method, including ascertaining that urine has at least partly ceased to flow downstream from a bladder of a subject through a conduit connected to a urinary catheter that catheterizes the subject, and in response to the ascertaining, increasing a pressure in the conduit.

There is further provided, in accordance with some embodiments of the present invention, a system including a display and a controller. The controller is configured to empty a bladder of a subject, by pumping urine from the bladder, to calculate an estimated amount of time from the emptying of the bladder required for a predefined volume to flow into the bladder, to receive a signal that varies as a function of a pressure within the bladder after the estimated amount of time from the emptying of the bladder, to ascertain the pressure within the bladder based on the signal, and to display, on the display, an output indicating that the pressure within the bladder is an intraabdominal pressure of the subject.

In some embodiments, the controller is configured to pump the urine through a urinary catheter that catheterizes the subject, and the signal is generated by a pressure sensor coupled to the urinary catheter.

In some embodiments, the controller is configured to calculate the estimated amount of time based on an amount of the urine pumped during a preceding period of time.

In some embodiments, the controller is further configured to verify that the pressure within the bladder is the intraabdominal pressure, by:
re-emptying the bladder, and
ascertaining that an amount of the urine pumped from the bladder during the re-emptying deviates from the predefined volume by less than a predefined threshold,
and the controller is configured to display the output in response to the verifying.

There is further provided, in accordance with some embodiments of the present invention, a method including emptying a bladder of a subject, by pumping urine from the bladder, calculating an estimated amount of time from the emptying of the bladder required for a predefined volume to flow into the bladder, after the estimated amount of time from the emptying of the bladder, receiving a signal that varies as a function of a pressure within the bladder, based on the signal, ascertaining the pressure within the bladder, and generating an output indicating that the pressure within the bladder is an intraabdominal pressure of the subject.

There is further provided, in accordance with some embodiments of the present invention, a fluid conduit including a first tube configured to carry urine downstream from a bladder of a subject and including one or more flexible walls configured to collapse into the first tube, as a pressure within the first tube decreases, until the first tube is closed, and a second tube coupled to the first tube and configured to carry the urine downstream from the first tube.

In some embodiments, the flexible walls include:
a first wall including a first face; and
a second wall including a second face coupled to the first face at opposing edges of the first face such that, as the pressure decreases, the first wall and second wall collapse toward one another until the first face and second face are fully in contact with one another between the edges.

In some embodiments, an upstream portion of the flexible walls is more flexible than is a downstream portion of the flexible walls.

There is further provided, in accordance with some embodiments of the present invention, a kit for fluid collection. The kit includes a tube having an upstream end for receiving a fluid output by a subject and having a downstream end, and a non-spill connector fixed to the downstream end of the tube to prevent outflow of the fluid and adapted to connect to a mating connector coupled to a fluid-collection bag, such that insertion of the mating connector into the non-spill connector opens the non-spill connector, whereby the fluid flows out of the tube through the connector and mating connector and into the fluid-collection bag.

In some embodiments, the upstream end of the tube is configured to receive urine from a urinary catheter.

In some embodiments, the non-spill connector includes multiple flexible leaves, which close together across the non-spill connector.

In some embodiments, the multiple flexible leaves include sections of a polymer diaphragm that extends across the non-spill connector.

In some embodiments, the kit further includes the mating connector and the fluid-collection bag, which is coupled to the mating connector so as to receive and store the fluid flowing out of the tube.

There is further provided, in accordance with some embodiments of the present invention, fluid collection apparatus, including a connector having an upstream end for connection to a urinary catheter and having a downstream end, a tube coupled to the downstream end of the connector so as to receive urine flowing through the catheter, and a temperature sensor configured to estimate a temperature of the urine flowing into the connector.

In some embodiments, the temperature sensor is functionally associated with the connector.

In some embodiments, the temperature sensor is configured to output an electrical signal that is indicative of the temperature of the urine, and the apparatus includes a wire, which is connected to the temperature sensor so as to convey the electrical signal to a measurement circuit.

In some embodiments, the temperature sensor is configured to output a pressure that is indicative of the temperature of the urine, and the apparatus includes a capillary tube, which is connected to the temperature sensor at an upstream end of the capillary tube, and is connected at a downstream end thereof to a pressure measurement device, which estimates the temperature responsively to a pressure in the capillary tube.

In some embodiments, the apparatus includes means for controlling the urine flow.

In some embodiments, the urine flow is stopped for a predefined time and the temperature is estimated at the end of that time.

In some embodiments, the predefined time is calculated based on the urine amount flowing through the connector during a predefined period prior to stopping the urine flow.

There is further provided, in accordance with some embodiments of the present invention, a pump, including a pumping mechanism, which is configured to propel a fluid through a tube, and a release mechanism, which is coupled to receive an indication of a malfunction in a fluid circuit to which the tube is connected, and, in response to the indication, to release the fluid from the tube.

In some embodiments, the indication of the malfunction includes a pressure increase in the tube at a location upstream of the pump.

In some embodiments, a part of the tube is flexible, and the pumping mechanism includes a plurality of rollers, which are configured to roll and press against the flexible part of the tube, and the pump includes a clamp, which is configured to press the flexible part of the tube against the pumping mechanism, so that the rollers compress the tube, and in response to the indication, the release mechanism is configured to release the clamp from the flexible part of the tube.

In some embodiments, the release mechanism includes a moveable rod having a first end in contact with the flexible part of the tube, and the indication of the malfunction causes a movement of the rod, which releases the clamp.

In some embodiments, the pump further includes a spring, which is connected to apply a compression against the clamp so as to press the clamp against the flexible part of the tube, and the release mechanism is configured to release the compression in the spring in response to the indication.

In some embodiments, the release mechanism includes an electromechanical element, which is configured to release the fluid in response to an electrical signal that is indicative of the malfunction.

There is further provided, in accordance with some embodiments of the present invention, a peristaltic pump, including a flexible tube, which is configured to receive a fluid from a fluid source, a plurality of pressing elements, which are configured to press sequentially against a part of the flexible tube, a clamp, which is configured to press the part of the flexible tube against the pressing elements, so that the pressing elements compress the tube, and one or more springs, which are coupled to apply a compression between the pressing elements and the clamp so that the pressing elements apply a force against the part of the flexible tube such that the force remains substantially constant irrespective of variations in mechanical characteristics of components of the pump.

In some embodiments, the one or more springs include a linear spring.

In some embodiments, the one or more springs include a coiled spring.

In some embodiments, the one or more springs are functionally associated with the clamp.

In some embodiments, the one or more springs are coupled to shift the pressing elements toward the clamp.

In some embodiments, the pump further includes a rotor, which includes a rotating drum, and the pressing elements include rollers, which are mounted on the drum, and the one or more springs are coupled to shift the rollers radially outward within the drum.

In some embodiments, the rollers are mounted on respective rods, which are configured to pivot about respective axes on the drum, and the one or more springs are coupled to exert a rotational force on the rods about the respective axes.

In some embodiments, the one or more springs are attached to the rods.

In some embodiments, the one or more springs are attached to the rollers.

In some embodiments, the rollers are mounted within respective radial slots in the drum, so that the compression applied by the one or more springs shifts the rollers radially within the radial slots.

In some embodiments, the rollers include rotational bearings, which are disposed at respective ends of the rollers and are configured to slide radially within the slots.

In some embodiments, the one or more springs are coupled to press the rotor toward the clamp.

In some embodiments, the pump further includes a lock, which is configured, upon insertion of the flexible tube into the pump, to permit the one or more springs to drive the pressing elements toward the clamp to a location at which the pressing elements apply the substantially constant force against the flexible tube, and then to lock the pressing elements in the location during operation of the pump.

In some embodiments, the pump further includes a lock, which is configured, upon insertion of the flexible tube into the pump, to permit the one or more springs to drive the clamp toward the pressing elements to a location at which the pressing elements apply the substantially constant force against the flexible tube, and then to lock the clamp in the location during operation of the pump.

There is further provided, in accordance with some embodiments of the present invention, a fluid collection or delivery apparatus. The apparatus includes a hanger configured to hold a fluid bag while the fluid bag receives fluid excreted from or perfuses fluid to a body of a subject, such that the fluid bag is suspended from the hanger, and a sensor coupled to the hanger and configured to sense a quantity of the fluid in the fluid bag.

In some embodiments, the fluid bag is coupled to receive urine from a urinary catheter. In some embodiments, the sensor is configured to measure a weight of the fluid in the fluid bag.

In some embodiments, the sensor is configured to measure a level of the fluid in the fluid bag.

In some embodiments, the apparatus further includes a controller, which is configured to issue an alarm when the quantity of the fluid reaches a predefined limit.

In some embodiments, the apparatus further includes a communication link coupled to convey an indication of the sensed quantity of the fluid to a receiver.

In some embodiments, the apparatus further includes a monitoring system, which is configured to receive the indication of the sensed quantity and to compute and display information regarding excretion of the fluid by the subject or fluid delivery to the subject over time.

In some embodiments, the monitoring system is configured to display data regarding one or more further fluids that are input to or output from the body of the subject.

In some embodiments, the monitoring system is configured to display data with respect to multiple subjects concurrently.

In some embodiments, the apparatus further includes a display.

The present invention will be more fully understood from the following detailed description of embodiments thereof, taken together with the drawings, in which:

### BRIEF DESCRIPTION OF THE DRAWINGS

FIG. 1A is a schematic illustration of a peristaltic pump, in accordance with some embodiments of the present invention;

FIG. 1B is a schematic illustration of a peristaltic pump tube, in accordance with some embodiments of the present invention;

FIG. 1C is a schematic illustration of a peristaltic pump mechanically coupled to a peristaltic pump tube, in accordance with some embodiments of the present invention;

FIG. 2 is a plot showing an example of an aging graph of a peristaltic pump tube;

FIGS. 3A-C are schematic illustrations of a urine reservoir in different respective states, in accordance with some embodiments of the present invention;

FIGS. 4A-C are schematic illustrations of a urine reservoir, in accordance with some embodiments of the present invention;

FIGS. 5A-C are schematic illustrations of the urine reservoir of FIGS. 4A-C in different respective states, in accordance with some embodiments of the present invention;

FIGS. 6A-C are schematic illustrations of a longitudinal cross-section through a urine reservoir in different respective states, in accordance with some embodiments of the present invention;

FIGS. 7A-C, FIGS. 8A-C, FIG. 9, and FIGS. 10A-C are schematic illustrations of optical sensors functionally coupled to a urine reservoir, in accordance with various different embodiments of the present invention;

FIGS. 11A-B are schematic illustrations of a contact sensor functionally coupled to a urine reservoir, in accordance with some embodiments of the present invention;

FIGS. 12A-B and FIG. 13 are schematic illustrations of a disposable kit, in accordance with various different embodiments of the present invention;

FIG. 14 is a schematic illustration of a catheter connector, in accordance with some embodiments of the present invention;

FIG. 15 is a schematic illustration of a dual-lumen tube, in accordance with some embodiments of the present invention;

FIG. 16 is a schematic illustration of the operation of a urine-pumping system, in accordance with some embodiments of the present invention;

FIG. 17 shows a flow diagram for the operation of the urine-pumping system per FIG. 16, in accordance with some embodiments of the present invention;

FIG. 18 shows a flow diagram for a control algorithm executed by a controller, in accordance with some embodiments of the present invention;

FIG. 19 is an example plot of a sensor signal as a function of time, in accordance with some embodiments of the present invention;

FIG. 20 is a schematic illustration of a urine-pumping system, in accordance with some embodiments of the present invention;

FIG. 21 is a schematic illustration of a disposable kit per FIG. 20, in accordance with some embodiments of the present invention;

FIG. 22 is a schematic illustration of a urine-pumping system, in accordance with some embodiments of the present invention;

FIG. 23 is a schematic illustration of a disposable kit per FIG. 22, in accordance with some embodiments of the present invention;

FIGS. 24A-C are schematic illustrations of a pressure valve, in accordance with some embodiments of the present invention;

FIG. 24D is a schematic illustration of a transverse cross-section through a prior-art tube in inflated and deflated states;

FIG. 24E is a schematic illustration of a transverse cross-section through a pressure-valve tube, in accordance with some embodiments of the present invention;

FIGS. 25-26 are schematic illustrations of a urine-pumping system, in accordance with different respective embodiments of the present invention;

FIG. 27 schematically illustrates an example performance of suction relief, in accordance with some embodiments of the present invention;

FIG. 28 is a schematic illustration of a urine-pumping system, in accordance with some embodiments of the present invention;

FIG. 29 shows a flow diagram for an algorithm for measuring intra-abdominal pressure, in accordance with some embodiments of the present invention;

FIG. 30 is a schematic illustration of a urine-pumping system, in accordance with some embodiments of the present invention;

FIG. 31 shows a block diagram of some components of a urine-pumping system, in accordance with some embodiments of the present invention;

FIG. 32 shows a flow diagram for a controlling-and-alerting algorithm executed by a controller, in accordance with some embodiments of the present invention;

FIG. 33 shows a plot tracking a reservoir volume over time, in accordance with some embodiments of the present invention;

FIG. 34A is a schematic side view of a replaceable fluid bag with a spill-proof connector, in accordance with some embodiments of the present invention;

FIG. 34B is a schematic detail view of the spill-proof connector of FIG. 34A;

FIGS. 34C and 34D are schematic frontal views of the spill-proof connector of FIG. 34A in closed and open configurations, respectively;

FIG. 35 is a schematic illustration of example displayed output, in accordance with some embodiments of the present invention;

FIGS. 36A and 36B are schematic side views of a peristaltic pump with a spring-loaded safety release in normal and released configurations, respectively, in accordance with some embodiments of the present invention;

FIG. 37 is a schematic illustration of a disposable kit comprising a pressure-regulating bypass tube, in accordance with some embodiments of the present invention;

FIGS. 38A and 38B are schematic side views of springs used in controlling pressure exerted by a clamp in a peristaltic pump, in accordance with some embodiments of the present invention;

FIGS. 39A, 39B and 39C are schematic side views of peristaltic pumps with spring-loaded pressure clamps, in accordance with embodiments of the invention;

FIG. 40 is a schematic side view of a catheter-tube connector with an integral temperature sensor, in accordance with some embodiments of the present invention;

FIG. 41 is a schematic side view of spring-loaded rollers of a peristaltic pump, in accordance with some embodiments of the present invention;

FIG. 42 is a schematic pictorial view of spring-loaded rollers of a peristaltic pump, in accordance with another embodiment of the invention;

FIG. 43 is a schematic side view of spring-loaded rollers of a peristaltic pump, in accordance with yet another embodiment of the invention;

FIG. 44A is a schematic detail view of a roller in a peristaltic pump with a spring-loaded rotational bearing, in accordance with some embodiments of the present invention;

FIG. 44B is a schematic pictorial view of spring-loaded rollers of a peristaltic pump, in accordance with a further embodiment of the invention;

FIGS. 45A and 46A are schematic pictorial and side views, respectively, of a peristaltic pump with spring-loaded rollers, in accordance with still another embodiment of the invention;

FIGS. 46A and 46B are schematic side views of a peristaltic pump with a replaceable cartridge before and after attachment of the cartridge to the pump, in accordance with some embodiments of the present invention;

FIG. 47A is a schematic side view of a hanging scale for a urine-collection bag, in accordance with some embodiments of the present invention;

FIG. 47B is a schematic detail view of the scale of FIG. 47A;

FIG. 47C is a schematic detail view of a controller that is integrated into the hanging scale of FIG. 47A;

FIG. 47D is a block diagram that schematically illustrates circuitry in the controller of FIG. 47C;

FIG. 48 is a schematic representation of a display screen showing a fluid-management dashboard, in accordance with some embodiments of the present invention;

FIG. 49 is a schematic illustration of a system for displaying urine-production parameters, in accordance with some embodiments of the present invention;

FIGS. 50-51 are schematic illustrations of a urine-pumping system, in accordance with different respective embodiments of the present invention;

FIGS. 52A-B are schematic illustrations of a conduit section, in accordance with different respective embodiments of the present invention;

FIGS. 53-54 are schematic illustrations of a control unit, in accordance with different respective embodiments of the present invention;

FIG. 55 is a schematic illustration of a control unit connected to power-supply box, in accordance with some embodiments of the present invention;

FIG. 56 is a schematic illustration of displayed output, in accordance with some embodiments of the present invention;

FIG. 57 is a schematic illustration of a control unit, in accordance with some embodiments of the present invention;

FIG. 58 is a schematic illustration of a urine-pumping system, in accordance with some embodiments of the present invention;

FIGS. 59-60 are schematic illustrations of a control unit, in accordance with different respective embodiments of the present invention;

FIG. 61 is a schematic illustration of a urine-pumping system, in accordance with some embodiments of the present invention;

FIG. 62 is a schematic illustration of a control unit, in accordance with some embodiments of the present invention;

FIGS. 63A-B and 64A-B are schematic illustrations of a reciprocating pump together with a conduit section, in accordance with various different embodiments of the present invention;

FIG. 65 is a schematic illustration of a urine-pumping system, in accordance with some embodiments of the present invention;

FIG. 66A is a schematic illustration of a disposable kit for facilitating measuring urine output and/or production, in accordance with some embodiments of the present invention;

FIG. 66B is a schematic illustration of a system for measuring urine output and/or production, in accordance with some embodiments of the present invention; and

FIG. 67 is a schematic illustration of a reciprocating pump together with a conduit section, in accordance with some embodiments of the present invention.

### DETAILED DESCRIPTION OF EMBODIMENTS

### Overview

Embodiments of the present invention include systems and methods for measuring a subject's urine output, i.e., a volume of urine excreted from the subject's bladder, and/or urine production, i.e., a volume of urine produced by the subject's kidneys, accurately and in real time. Embodiments of the present invention further include systems and methods for communicating and/or displaying the urine output or production, or any relevant parameter derived therefrom, such as a time-varying rate of urine production. Embodiments of the present invention further include systems and methods for measuring other parameters such as intra-abdominal pressure (IAP), core body temperature, and urine optical parameters such as opacity.

In some embodiments, a urine-pumping system is configured to measure the subject's urine output and/or production. The urine-pumping system comprises a disposable apparatus, referred to herein as a “kit,” along with a non-disposable urine-pumping device. The disposable kit, which is for one-time use, comprises a urine conduit configured to connect a urinary catheter (e.g., a Foley catheter), which catheterizes the bladder of the subject, to a urine-collection bag. The urine-pumping device comprises a controller (i.e., control circuitry) configured to pump urine through the conduit using a positive-displacement pump, typically so that the bladder remains substantially void of urine. Based on the pumping, the controller calculates the subject's urine output and/or production and, typically, the subject's rate of urine production. Optionally, the controller may display the rate of urine production (and/or other related parameters), and/or communicate the rate (and/or other related parameters) to one or more other devices or systems such as a patient monitor, a nurse station monitor, or an electronic medical record (EMR).

In general, any type of positive-displacement pump may be used. Typically, the positive-displacement pump comprises one or more force-applying elements configured to apply force to the conduit, thereby pumping urine through the conduit. At least some of these elements may belong to the non-disposable urine-pumping device; in such embodiments, the non-disposable force-applying elements of the pump may be calibrated, and the calibration parameters may be stored in a non-volatile memory in the device. Alternatively or additionally, at least some of the pump elements may belong to the disposable kit.

Typically, the force-applying elements comprise an actuator. In the context of the present application, including the claims, the term “actuator” may include any device that uses power (e.g., electrical, mechanical, pneumatic, or hydraulic power) supplied to the actuator to cause movement of another element.

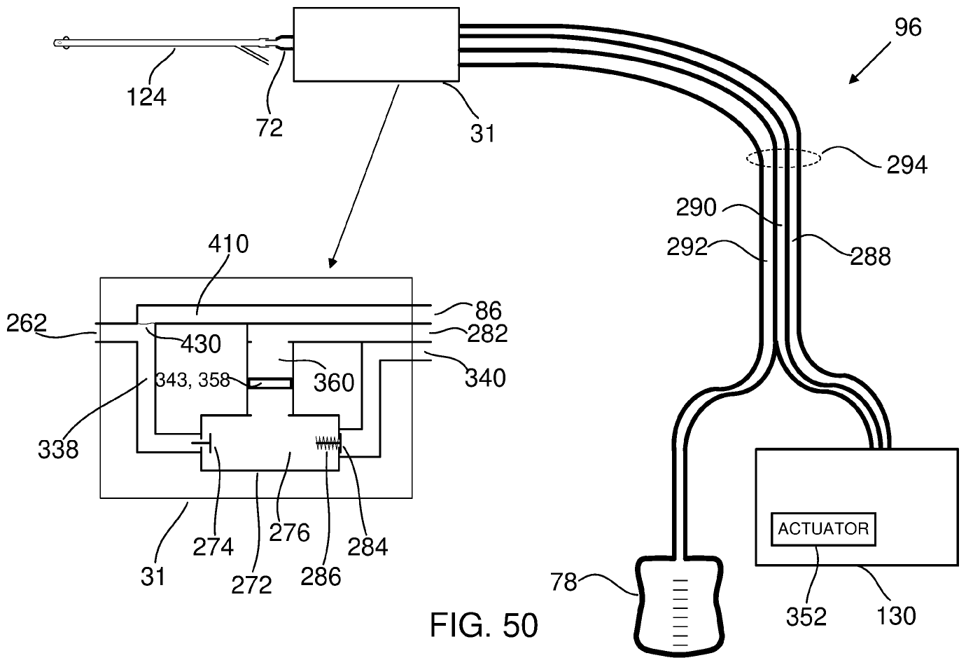

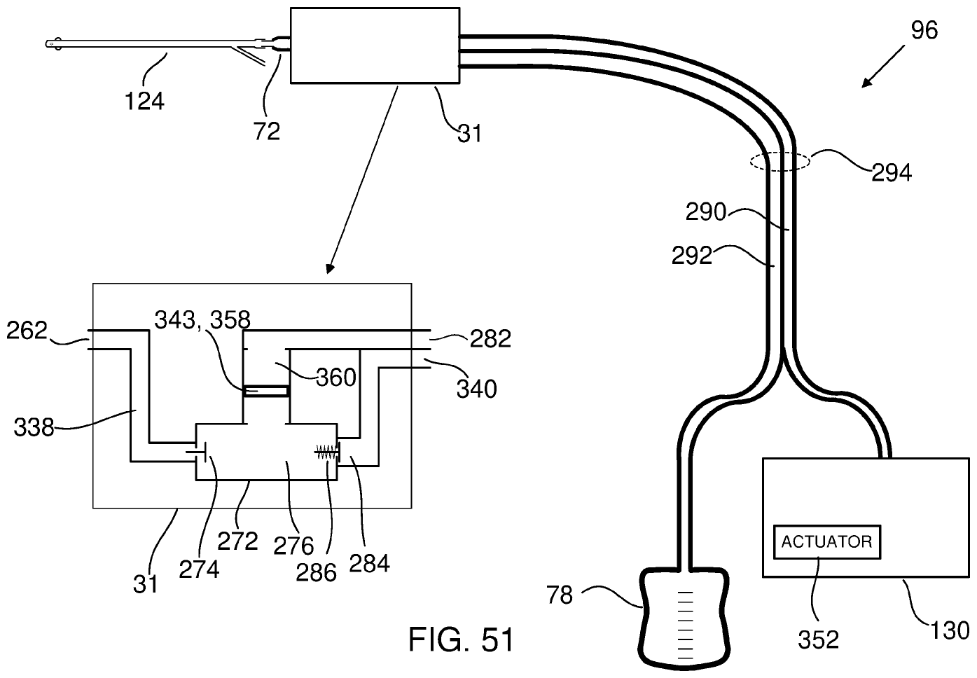

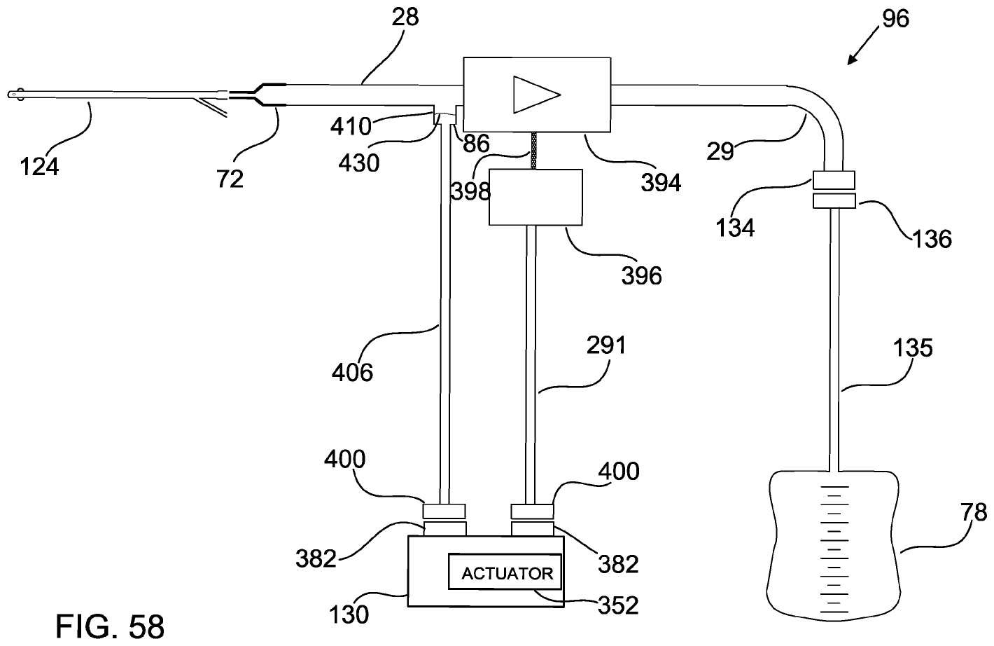

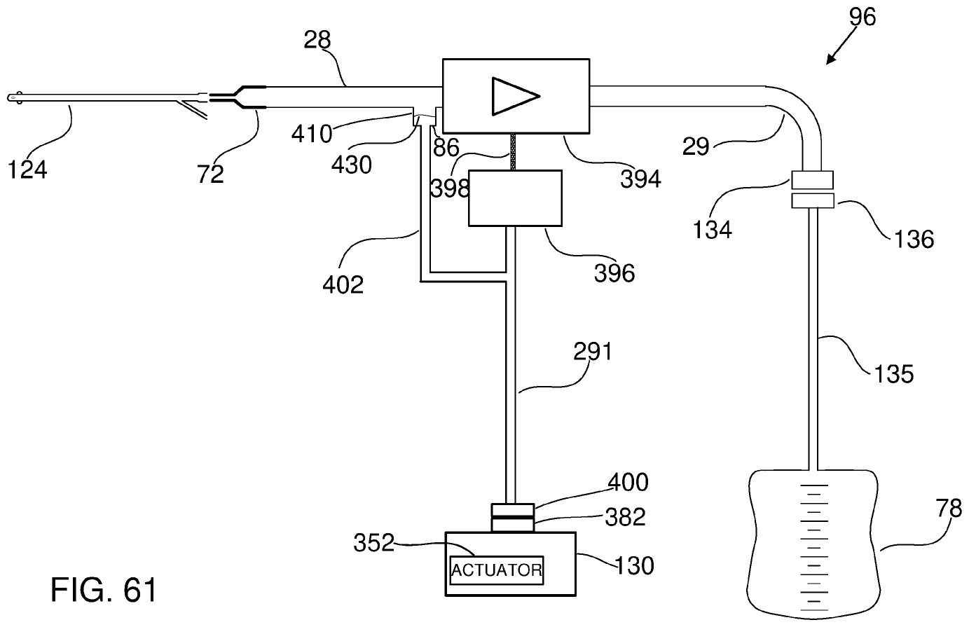

For example, FIGS. 50-51, 58, and 61 show embodiments in which an actuator reversibly couples to the conduit, and applies force to the conduit, via a fluid (i.e., a gas or liquid) contained within a tube. In these embodiments, the actuator converts the supplied power into pneumatic or hydraulic power, which in turn causes movement of a moveable wall of the conduit (FIGS. 50-51) or movement of a shaft (FIGS. 58 and 61).

Alternatively, the actuator may be coupled to a pressing element configured to press the conduit (while in contact with the conduit) when actuated by the actuator. In such embodiments, the actuator uses the supplied power to move the pressing element.

For example, in some embodiments, the conduit comprises a peristaltic pump tube, and the pressing element comprises a rotor or an array of linear translational elements configured to squeeze urine from the peristaltic pump tube in a desired direction when actuated by the actuator. In some such embodiments, to facilitate a more accurate computation of urine flow, the system comprises one or more additional components (e.g., springs) that render the pumping force applied to the peristaltic pump tube independent from manufacturing tolerances in, and/or wear over time in, one or more components of the system. Such components may include the tube, a clamp that clamps the tube to the pressing element, and/or the pressing element.

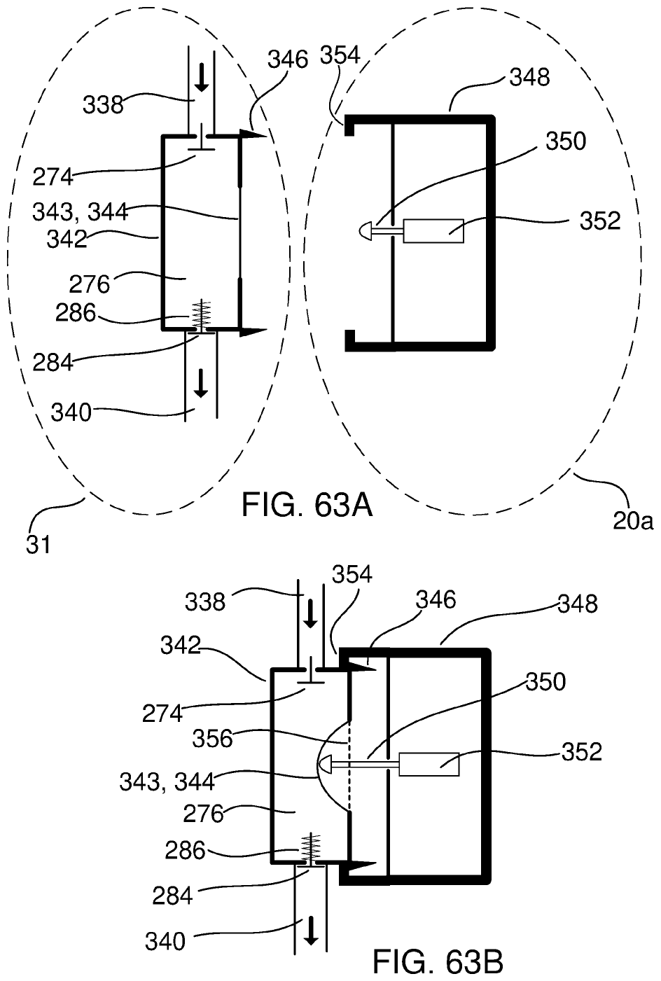

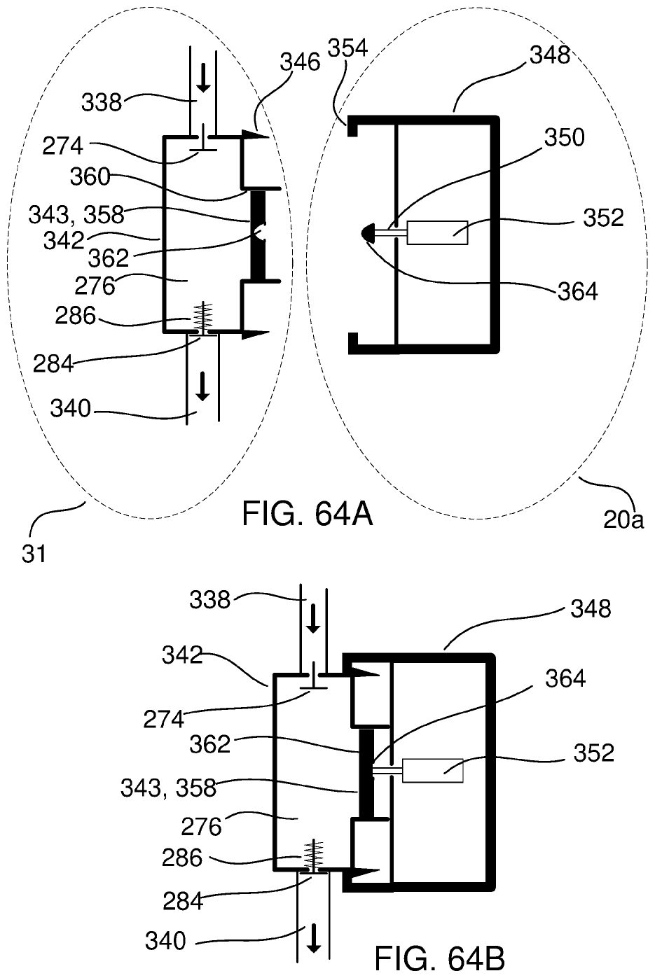

As another example, as shown in FIGS. 63A-B and 64A-B, the conduit may comprise a pump chamber having a moveable wall (e.g., a diaphragm or piston), and the pressing element may comprise a plunger configured to press against the moveable wall when actuated by the actuator, thereby forcing urine from the pump chamber in a desired direction.

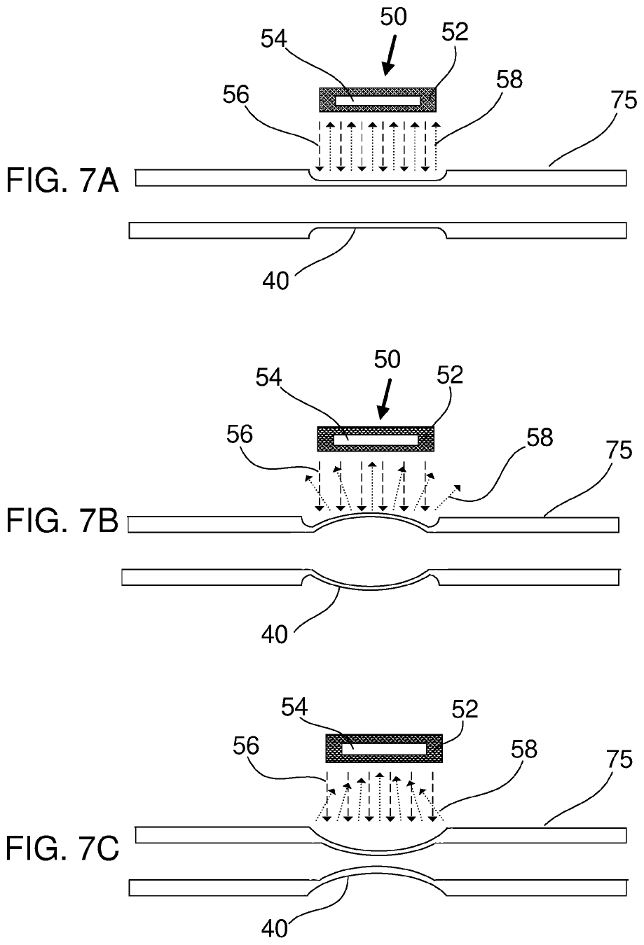

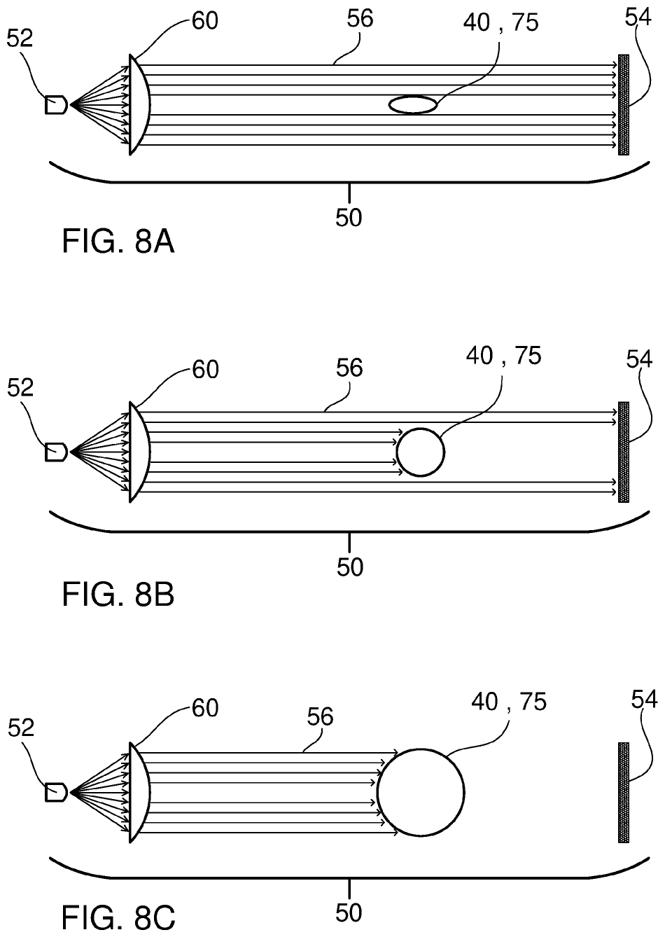

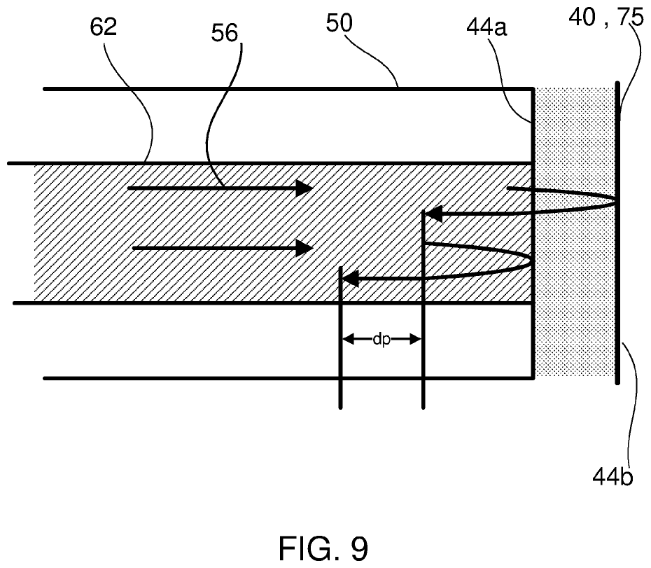

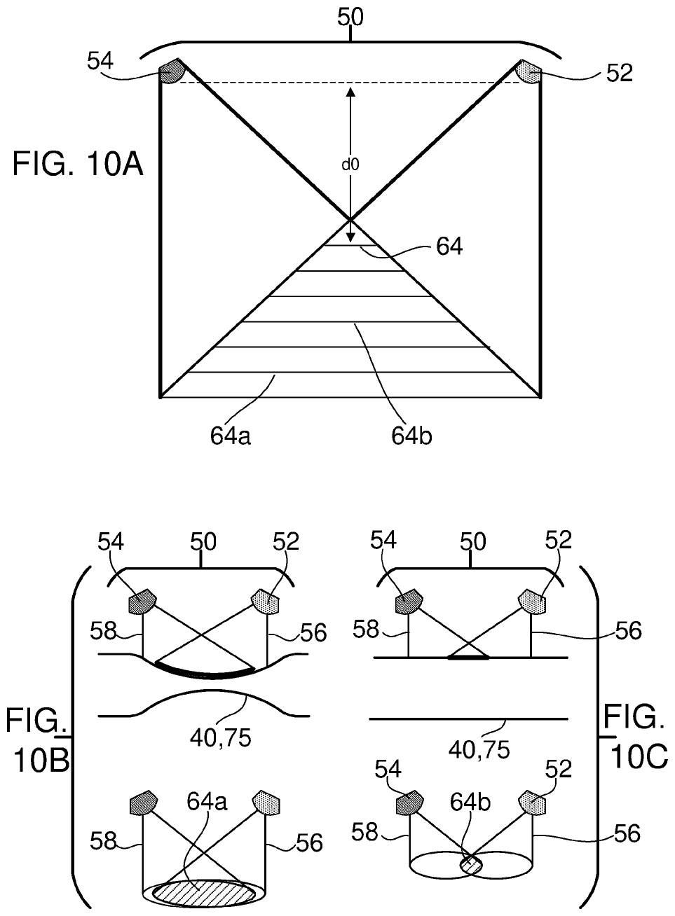

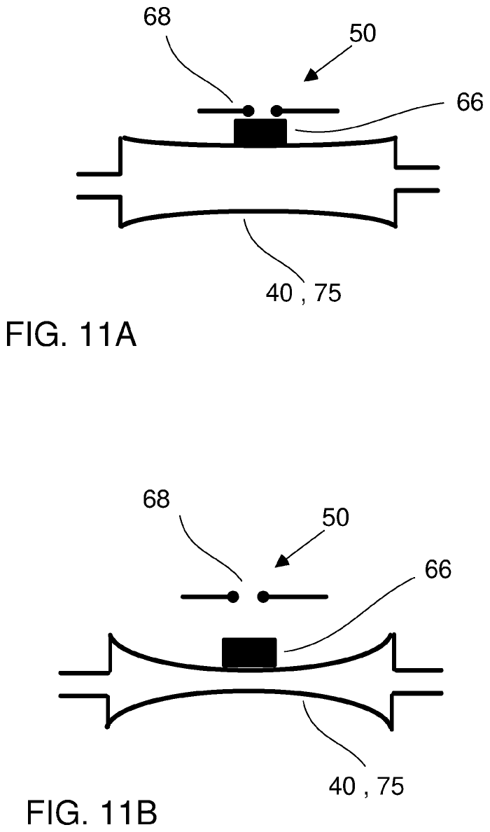

In some embodiments, the system further comprises a sensor for facilitating control of the pump. In particular, the sensor is configured to communicate, to the controller, a signal that varies as a function of the amount of the urine in the conduit or in the subject's bladder, and the controller is configured to control the force-applying elements of the pump responsively to the signal. The sensor may comprise, for example, a pressure sensor, a volume sensor, an optical sensor (FIGS. 7-10), a capacitive sensor, a resistive sensor, an inductive sensor, an ultrasonic sensor, and/or a contact sensor (FIGS. 11A-B). The sensor may sense the displacement of a diaphragm, an expandable reservoir wall, or the wall of another expandable portion of the conduit. The displacement may be sensed optically, resistively, capacitively, inductively, by ultrasound, by contact, magnetically, or in any other suitable way.

A capacitive sensor may be implemented in several ways. For example, the wall or diaphragm may be coated with a conducting material that serves as one plate of a capacitor, another plate may be fixed near the first plate, and both plates may be electrically connected to a circuit that measures the capacitance. As the wall or diaphragm is displaced, the distance between the plates, and hence, the capacitance, changes, such that the capacitance is indicative of the displacement.

In some embodiments, the capacitor belongs to an oscillator, such that the oscillator's frequency depends on the capacitor's capacitance. By measuring the oscillator's frequency (e.g., by counting the number of cycles per a given time period), the capacitor's capacitance may be ascertained.

A resistive sensor may also be implemented in several ways. For example, the wall or diaphragm may be coated with a resistive material and electrically connected at two opposing edges to an electrical circuit, such that the wall or diaphragm functions as a resistor in the circuit. The resistance of the resistor may be determined in a similar way to that described above for the capacitive sensor (e.g., by determining the frequency of an oscillator). As the wall or diaphragm is displaced, it stretches, and thus, the resistance between the two opposing contact points changes. Hence, the change in resistance is indicative of the displacement of the wall or diaphragm.

An inductive sensor may be implemented in several ways. For example, the wall or diaphragm may be coated with a ferromagnetic material and placed at the spine of a coil that may belong to an electrical circuit. As the wall or diaphragm is displaced, the inductance of the coil changes, and hence, the inductance is indicative of the displacement of the wall or diaphragm. The inductance of the coil may be determined in a similar way to that described above for the capacitive sensor (e.g., by determining the frequency of an oscillator).

In some embodiments, the sensor comprises an ultrasound transducer disposed near the wall or diaphragm. The round-trip-delay of an ultrasonic signal (wave) may be measured to determine the distance of the wall or diaphragm from the transducer.

A magnetic sensor may comprise a magnetometer, and the wall or diaphragm may be coated with a metallic material. The wall or diaphragm displacement may be ascertained from the intensity of the magnetic field measured by the magnetometer.

In some embodiments, the conduit comprises an expandable portion configured to expand as urine flows into the expandable portion. The sensor is configured to sense a degree of expansion of the expandable portion and to communicate a signal, indicating the degree of expansion, to the controller. For example, a pressure sensor may sense a pressure that varies with the degree of expansion, such as the pressure in a fluid-filled tube or chamber that is separated, by a flexible diaphragm, from the expandable portion or a portion of the conduit near the expandable portion. As another example, an optical sensor may sense an amount of light reflected from the expandable portion, this amount varying with the degree of expansion. Responsively to the degree of expansion, the controller controls the force-applying elements of the pump.

In some such embodiments, the expandable portion comprises a urine reservoir disposed upstream from the portion of the conduit to which force is applied by the force-applying elements. The reservoir may comprise at least one moveable (e.g., flexible) wall, the position and/or shape of which varies with the amount of urine in the reservoir. Optionally, the reservoir and pump may be manufactured together as part of a single integrated disposable unit.

In other such embodiments (e.g., as shown in FIGS. 50-51, 52A-B, 63A-B, and 64A-B), the expandable portion comprises a pump chamber comprising a moveable wall, such as a diaphragm or piston, to which force is applied by the force-applying elements. The pump chamber is configured to expand, via movement of the moveable wall, as urine flows into the pump chamber.

Typically, the controller is contained in a control unit, which may be conveniently coupled to the subject's bedside. Other components of the urine-pumping device may be contained in the control unit or remotely therefrom.

In some embodiments, the control unit also comprises a pump pressing element. In such embodiments, a portion of the conduit may be coupled to (e.g., inserted into) the control unit such that the conduit is coupled to the pressing element. Optionally, the coupled (e.g., inserted) portion of the conduit may comprise a reservoir, and the control unit may comprise a sensor configured to monitor the reservoir.

In other embodiments, though the control unit comprises the actuator for the pump, the pressing element is external to the control unit. Alternatively, the pump may lack a pressing element, in that the actuator may pump the urine by applying pneumatic or hydraulic force to the conduit. In such embodiments, the actuator may be coupled to the pressing element or to the conduit via wires and/or tubes.

For example, a pumping force may be applied to the conduit through a fluid-filled tube. Optionally, the control unit may further comprise a pressure sensor connected to the tube, and the controller may control the pumping force responsively to the pressure sensed by the pressure sensor when the pumping force is not applied. Thus, advantageously, a single tube may be used (alternatingly) for both sensing and pumping.

In yet other embodiments, the actuator is external to the control unit, and is powered via electrical wiring running from the control unit.

In general, the pump may be actuated electrically, by hydraulic or pneumatic force, or by mechanical force. The pump actuator may comprise a motor (e.g., an electric motor, a hydraulic motor, or a pneumatic motor), a solenoid, or a hydraulic or pneumatic piston, for example.

In some embodiments, instead of a reservoir as described above, the conduit comprises a thin membrane (also referred to herein as a “diaphragm”) coupled to the inlet of a pump chamber, which is deflected as urine flows into or out of the pump chamber. In such embodiments, a pressure sensor may measure a fluid pressure—i.e., a pneumatic or hydraulic pressure—that varies as the membrane is deflected. Alternatively, an optical sensor may sense the deflection of the membrane by emitting light at the membrane.

In some embodiments, the urine-pumping device and/or the disposable kit further comprises a sensor (e.g., a pressure sensor) coupled to the inlet of the pump, and/or a sensor (e.g., a pressure sensor) coupled to the outlet of the pump. In such embodiments, the controller may control the pumping responsively to signals from any of these sensors.

In some embodiments, the disposable kit comprises a machine-readable data-storage medium such as a barcode, a quick response (QR) code, a volatile memory, a non-volatile memory (e.g., a flash memory, a read-only memory (ROM), or an electrically erasable programmable read-only memory (EEPROM)), a radio frequency identification (RFID) tag, a flash memory, and/or machine-readable printing or engraving. The data-storage medium may store various parameters such as pump-tube characteristics, calibration parameters, security parameters, subject-specific parameters (e.g., the subject's ID), or measurement values. Some of these parameters (e.g., pump-tube characteristics) may be stored, printed, or engraved during the manufacture of the disposable kit.

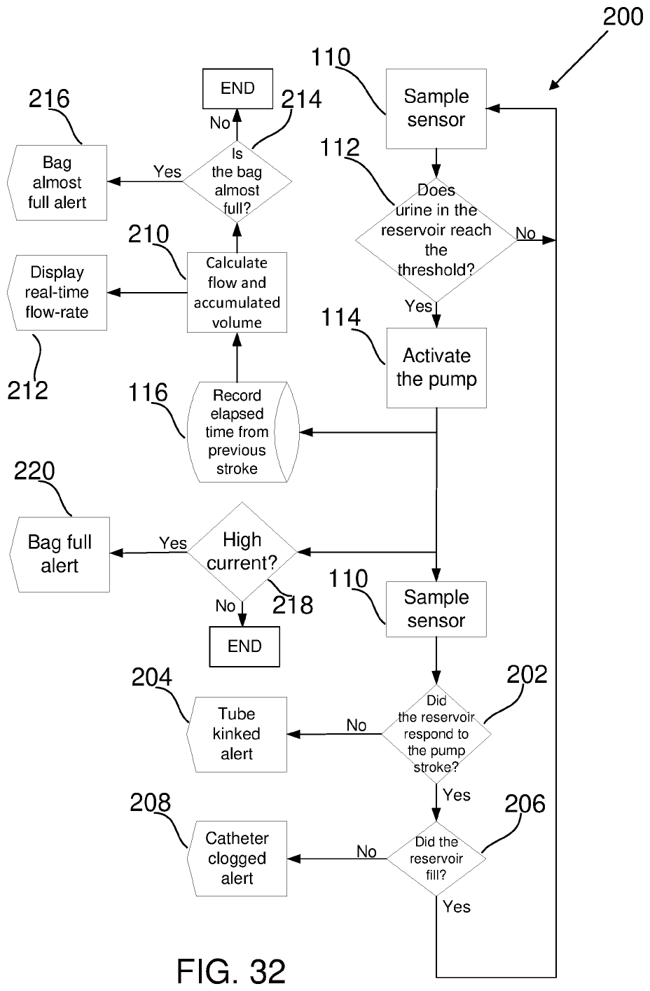

In some embodiments, the controller is configured to generate alerts, e.g., as described below with reference to FIG. 32. For example, the controller may generate an alert indicating an impeded flow of urine upstream or downstream from the pump, an alert indicating that the urine-collection bag is almost full, or an alert indicating a failure of the pump.

In some embodiments, the system comprises a suction-relief mechanism configured to relieve the bladder from any excess suction forces that cause the bladder tissue to be sucked into the urinary catheter. In some such embodiments, the suction-relief mechanism comprises a pressing element, such as a plunger, configured to squeeze a portion of the conduit upstream from the pump. To facilitate this squeezing, this portion of the conduit may be more flexible than other portions of the conduit, e.g., by virtue of having a thinner wall.

In other embodiments, suction relief is performed by operating the pump in the reverse (upstream) pumping direction.

In some embodiments, the urine-pumping device comprises one or more batteries. The batteries, which may be rechargeable or non-rechargeable, may power the control unit when the control unit is disconnected from the main power, e.g., when the subject is moved to a different bed or is taken for an intrabody image.

For embodiments in which the batteries are rechargeable, the system may comprise battery-charging circuitry configured to charge the batteries when the control unit is connected to the main power supply. The battery-charging circuitry may also ascertain the battery charge level, monitor the battery temperature, and adjust the charging accordingly if the temperature is too high. Alternatively or additionally, the battery charger may monitor the battery health and send a signal to the controller when a battery needs to be replaced.

Alternatively or additionally, the urine-pumping device may comprise a power supply for supplying power to all the system components. In some such embodiments, the power supply is integral with the control unit. In other such embodiments, the power supply is separate from the control unit and is connected to the control unit by an electric cable. For example, the power supply may be in a box configured to couple to the wall. Optionally, the power-supply box may also comprise communication circuitry and/or communication ports, and the cable may comprise communication wires in addition to power wires. An advantage of having the communication circuitry and/or ports belong to the power-supply box, rather than to the control unit, is that it is relatively simple to connect or disconnect the control unit when moving the bed or replacing the control unit, given that only a single cable is connected to the control unit.

In general, the controller may be configured for performing various tasks. For example, the controller may be configured to communicate with a sensor upstream or downstream from the pump, calibrate the sensor, and/or control the sensor. Alternatively or additionally, the controller may control a pump actuator, a suction-relief mechanism, and/or a battery charger. Alternatively or additionally, the controller may control a display, display data on the display, control a touch screen, and/or receive commands from the touch screen. Alternatively or additionally, the controller may communicate with one or more external devices or systems such as a patient monitor, an EMR, or a device (e.g., a mobile phone or tablet) of the subject's physician.

More specifically, in some embodiments, the controller is configured to execute a pumping algorithm, per which the controller decides when to activate the pump and, optionally, how much urine to pump during each activation. The controller is further configured to log the number of strokes that were pumped during each activation, and to calculate the amount of pumped urine based on various parameters such as the number of pumped strokes during the activation, the number previously-pumped strokes for the conduit, elapsed times between strokes, the total duration of the strokes, the ambient temperature, the temperature of the urine, the pump inlet pressure, the pump outlet pressure, calibration parameters, and/or manufacturing parameters of the conduit. For example, for a rotary peristaltic pump, the pumped volume may be calculated based on the number of rotations (including fractional rotations) of the pump rotor and the respective volumes pumped during the rotations. For a linear peristaltic pump, the pumped volume may be calculated based on the number of times the translational elements of the pump pressed on the pump tube. The controller may further calculate the instantaneous urine flow rate (which, assuming the volume in the bladder is kept relatively constant, i.e., within a relatively small range, is generally equal to the instantaneous rate of urine production) by dividing the pumped volume by the elapsed time from the previous pump activation.

Alternatively, the controller may communicate basic pumping information (e.g., the number and/or times of executed strokes and/or stroke volumes) to another computer processor, and the latter processor may calculate total urine production, rates of urine production, and/or any other relevant parameters.

In some embodiments, the controller activates the pump in response to ascertaining, based on a signal from a sensor, that a pumping threshold was reached. For example, based on the sensor signal, the controller may ascertain that the urine volume in the reservoir or the urine pressure in the conduit exceeds a predefined value.

Optionally, following the activation of the pump, the controller may stop the pump in response to ascertaining, based on the sensor signal, that a stopping threshold was reached. For example, the controller may ascertain, based on the sensor signal, that the urine volume in the reservoir or the urine pressure is below a predefined value. Alternatively, the controller may cause the pump to execute a predefined number of strokes, such that the pump stops after the strokes are executed. (The number of strokes may be based, for example, on the elapsed time from the most recent stroke.)

In yet other embodiments, the controller operates the pump so as to keep a parameter, such as the volume in the reservoir or the pressure in the conduit, as close as possible to a predetermined value. This may be done, for example, using a Proportional Integral Derivative (PID) algorithm. In such embodiments, the pumped volume may be calculated based on any of the parameters described above (e.g., the number of strokes and the respective volume of each stroke), or based on the number of rotations of the pump rotor and the speed of rotation.

In the event that a series of multiple strokes is performed, the strokes may share the same movement profile; for example, in the case of a peristaltic pump, during each stroke, the rotor may accelerate, remain at a constant speed, and then decelerate. Alternatively, some strokes may have different respective movement profiles so as to achieve a more continuous pumping; for example, in the case of a peristaltic pump, the rotor may accelerate at the beginning of the first stroke, turn at a constant speed (or at a varying speed, e.g., per a PID algorithm), and then decelerate at the end of the last stroke.

In some embodiments, the controller is further configured to execute a suction relief algorithm, per which the controller decides when and how to perform suction relief, and executes the suction relief. In some embodiments, suction relief is performed when a pressure sensor upstream from the pump does not show any pressure increase for a predetermined period of time, indicating that the outflow of urine through the catheter is likely blocked, e.g., by the bladder tissue.

In some embodiments, the controller is further configured to read data from the disposable kit. In some embodiments, these data include physical parameters of the conduit, which the controller may use for calculating the volume of urine flow. For example, the controller may calculate the stroke volume based on the inner and outer diameters and the hardness of the peristaltic pump tube. Alternatively or additionally, these data may include a disposable-kit identifier (e.g., a serial number), which the controller may use to associate the disposable kit with a particular subject. Thus, even if the disposable kit is disconnected from the control unit and later reconnected, the controller may identify the subject from whom urine is being pumped. Moreover, provided the control units in the hospital are configured to communicate relevant urine-flow data to an EMR or another centralized information-management system, the subject may be moved, together with subject's disposable kit, from a first control unit to a second control unit, given that the second control unit may use the disposable-kit identifier to retrieve, from the EMR, any urine-flow data calculated by the first control unit.

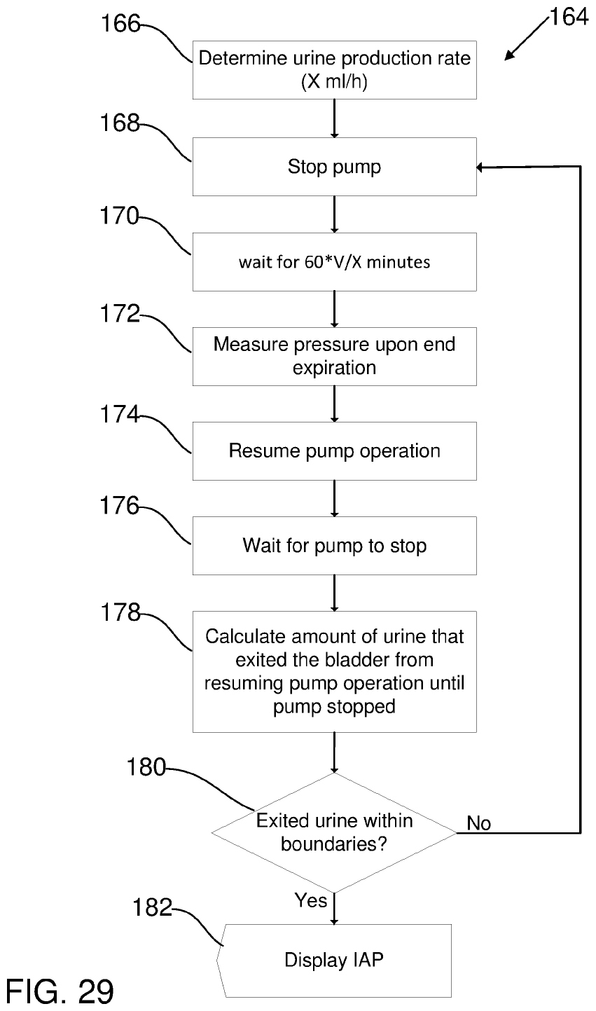

In some embodiments, the controller is further configured to execute an IAP-measurement algorithm as described, for example, with reference to FIG. 29.

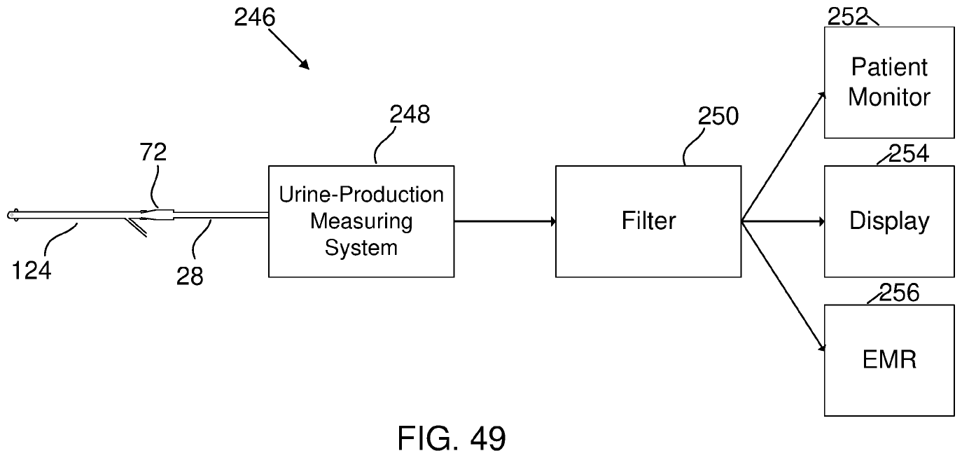

In some embodiments, the controller is further configured to execute a filtering algorithm for filtering noise from the urine-production signal, thereby producing a clean urine-production signal, as described, for example, with reference to FIG. 49.

Alternatively or additionally to facilitating control of the pumping, a sensor may be used to estimate the IAP of the subject. Alternatively or additionally, a pressure reading from a pressure sensor upstream from the pump and/or a pressure sensor downstream from the pump may be used in the calculation of the urine flow, given that the pressure in the conduit upstream and/or downstream from the pump may influence the pump stroke volume. Alternatively or additionally, sensor readings may be used to identify an impeded flow of urine upstream or downstream from the pump, e.g., due to a blockage in the conduit or in the urinary catheter, or due to the urine-collection bag being full.

Alternatively to using a sensor, the pressure downstream from the pump may be estimated by measuring the electric current consumed by the pump actuator during pumping, as the amount of current increases with the downstream pressure.

In some embodiments, the conduit comprises a tube having a single lumen for urine flow. In other embodiments, the tube has two lumens, one for urine flow and another for electrical wires that carry electric power and/or signals between the control unit and any other component such as a pressure sensor, a temperature sensor, or a pump actuator. Alternatively, the second lumen may contain a gas or liquid (e.g., oil) for sensing pressure or temperature, and/or for applying pneumatic or hydraulic force. Alternatively, the second lumen may contain a wire or thread for applying mechanical force for actuating a pump.

As another alternative, the tube may have three lumens: one for urine, another for transferring electric power and/or signals or for applying force (as described above), and a third containing a fluid (i.e., a gas or liquid) for pressure or temperature sensing.

As yet another alternative, the tube may have four lumens: one for urine, another for transferring electric power and/or signals, another containing a fluid for applying force, and a fourth containing a fluid for pressure sensing.

In some embodiments, the disposable kit further comprises any one or more of the urinary catheter, a catheter connector for connecting the urinary catheter to the conduit, a temperature sensor, a urine sampling port, and the urine-collection bag. The urine-collection bag may comprise a bottom valve for emptying the bag, such that the bag need not be replaced. Alternatively or additionally, the collection bag may comprise an inlet connector via which the bag may be disconnected from a complementary tube connector (described immediately below), thereby facilitating replacement of the bag. In some embodiments, the urine-collection bag further comprises a one-way inlet valve, which inhibits spilling of urine from the bag.

In some embodiments, the disposable kit comprises a tube segment through which urine flows to the urine-collection bag. The tube segment may be permanently connected to the urine-collection bag. Alternatively, the tube segment may comprise a tube connector at its end, the tube connector being configured to mate with the aforementioned inlet connector of the urine-collection bag. In some embodiments, the tube connector comprises a non-spill connector, which inhibits spilling of urine when the tube segment is not connected to the bag.

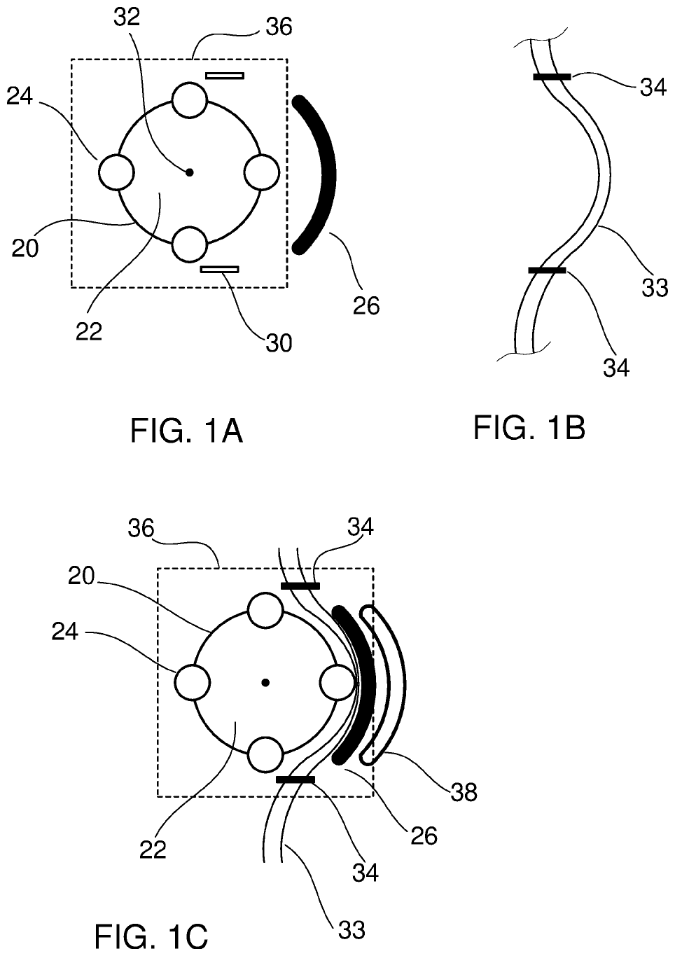

In general, it is noted that the disposable kit may comprise any of the system components described herein, even those components that do not contact the urine. For example, the disposable kit may comprise a peristaltic pump tube along with clamp 26 (FIG. 1A) and/or tube anchors 34 (FIG. 1B). Alternatively or additionally, parts of the peristaltic pump itself, such as the rotor and/or rollers, may belong to the disposable kit. Alternatively or additionally, for any type of pump, the actuator of the pump may belong to the disposable kit.

It is noted that each of the features described above may be implemented in any one of the embodiments described below with reference to the figures, as applicable.

In the context of the present application, including the claims, a “reversible coupling” of two items to one another refers to any coupling that may be undone without the use of any tools (and without breaking any of the items).

Some advantages of embodiments of the present invention include the following:
1. Given that the urine is pumped, the flow of urine from the bladder does not rely on the force of gravity. Therefore, the control unit and urine-collection bag may be placed at any height. (In contrast, in a gravity-based system, it might be necessary to place the collection bag on the floor, where it might become contaminated.) For example, the control unit may be coupled to the railing of the subject's bed, which is generally at a convenient height, and the urine-collection bag may be raised from the floor.
2. Given the lack of reliance on gravity as described above, the conduit may have any length. (In contrast, in a gravity-based system, it may be necessary to limit the length of the tube that drains the bladder.) Thus, for example, the control unit may be placed behind or at the foot of the subject's bed, and the conduit may pass from the urinary catheter to the control unit. In this regard, it is noted that there are several advantages to placing the control unit behind or at the foot of the bed. For example, the control unit is less likely to strike against an object (e.g., a doorpost) when the subject's bed is moved. Moreover, due to the greater length of the conduit, the subject may be treated or turned over without any tangling of the conduit.
3. The bladder may be continually emptied by the pump, such that the amount of urine output from the bladder serves as an accurate proxy for the real-time urine production by the kidneys. In contrast, in gravity-based systems, the bladder may retain a significant amount of urine, such as ˜100 ml of urine on average, and this amount may change over time. Hence, in gravity-based systems, the urine output from the bladder may not serve as an accurate proxy for the real-time urine production by the kidneys. Moreover, the residual urine increases the risk of catheter-associated urinary tract infections.
4. The urine-pumping system described herein may be configured to overcome certain factors that may inhibit the release of urine from the bladder, including blockages in the conduit and/or suction of the bladder into the eyelets of the urinary catheter.

### System Description

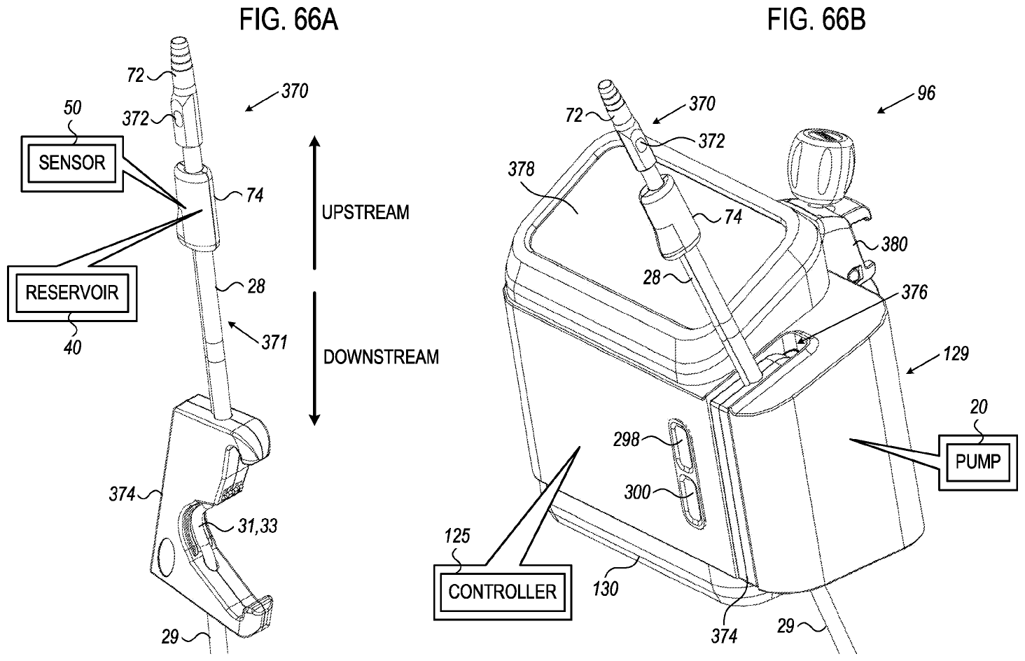

Reference is initially made to FIG. 66A, which is a schematic illustration of a disposable kit 370 for facilitating measuring urine output and/or production, in accordance with some embodiments of the present invention. Reference is also made to FIG. 66B, which is a schematic illustration of a system 96 (referred to herein as a “urine-pumping system”) for measuring urine output and/or production, in accordance with some embodiments of the present invention.

System 96 comprises kit 370 along with a non-disposable urine-pumping device 129. Kit 370 comprises a fluid conduit 371 configured for the flow of urine therethrough. Conduit 371 comprises at least one tube, which is configured to carry urine that flows downstream from a bladder of a subject via a urinary catheter (e.g., a Foley catheter) that catheterizes the subject. Conduit 371 further comprises a conduit section 31, which is coupled to the tube in fluid communication with the tube. In some embodiments, kit 370 further comprises a cartridge 374 (which may also be referred to as a “cassette”) or another type of housing, which contains conduit section 31.

Device 129 comprises one or more force-applying elements. The force-applying elements are configured to reversibly couple to conduit 371 (in particular, to conduit section 31), and to apply force to the conduit (in particular, to conduit section 31) when coupled to the conduit. As the force is applied, urine is squeezed from the conduit section in a downstream direction, i.e., away from the subject's bladder.

In some embodiments, the force-applying elements of the urine-pumping device comprise a pressing element, i.e., an element configured to apply force to conduit section 31 by pressing against the conduit section, along with an actuator configured to actuate the pressing element. For example, conduit section 31 may comprise a peristaltic pump tube 33, and the urine-pumping device may comprise a peristaltic pump 20 comprising a rotor or one or more linear translational elements configured to press against peristaltic pump tube 33. Alternatively, conduit section 31 may comprise a pump chamber comprising a moveable wall (e.g., a diaphragm wall or piston wall), and the urine-pumping device may comprise a plunger configured to press against the moveable wall, e.g., as described below with reference to FIGS. 63A-B and 64A-B.

In other embodiments, the force-applying elements comprise an actuator configured to apply pneumatic or hydraulic force to the conduit section via a fluid-filled tube. In such embodiments, the conduit section may comprise a pump chamber comprising a moveable wall, and the force may be applied to the moveable wall. For example, as described below with reference to FIGS. 50-51 and 52A-B, the conduit section may comprise a diaphragm wall or piston wall to which force is applied via the fluid in the tube.

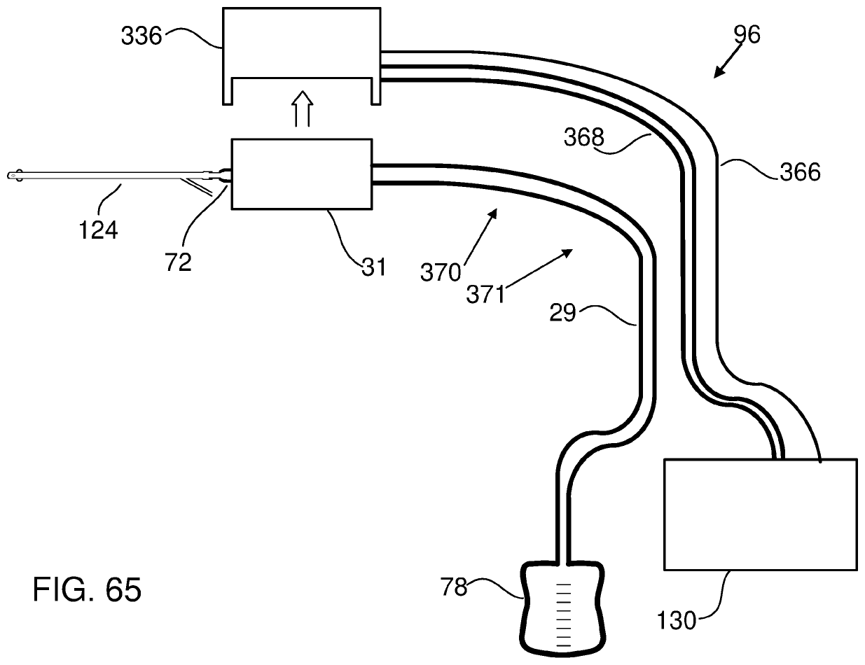

In some embodiments, as shown in FIG. 66A, conduit 371 comprises both an upstream tube 28, which is connected to the upstream end of conduit section 31 and therefore carries urine to the conduit section, and a downstream tube 29 (also referred to hereinbelow as an “exit tube”), which is connected to the downstream end of the conduit section and therefore carries urine from the conduit section. (Thus, conduit 371 may comprise at least three tubes in fluid communication with each other: upstream tube 28, peristaltic pump tube 33, and exit tube 29.) The downstream end of exit tube 29 is connected to a urine-collection bag 78 (shown in FIG. 65, for example), which may also belong to kit 370.

In other embodiments (e.g., as shown in FIGS. 50-51 and 65), conduit 371 does not comprise upstream tube 28.

Device 129 further comprises a controller 125 (which may be alternatively referred to as a “processor”), configured to control the pumping of urine through the conduit and perform other functions described herein.

In some embodiments, conduit 371 further comprises an expandable portion configured to expand as urine flows into the expandable portion. A sensor 50, which may belong to kit 370 or to the urine-pumping device, is configured to sense the degree of expansion of the expandable portion, and to generate a signal indicating the degree of expansion. The signal is communicated to controller 125, which pumps the urine through the conduit in response to the signal and, optionally, in response to other parameters, as detailed below in the section entitled “Pump control.”

For example, conduit 371 may comprise an expandable reservoir 40 disposed upstream from conduit section 31. For example, reservoir 40 may be coupled to the upstream end of tube 28, such that the urine flows from the reservoir into tube 28. In such embodiments, sensor 50 may be configured to communicate, to the controller, a signal that varies as a function of the amount of urine in the reservoir. Optionally, the reservoir and sensor may be housed in a housing 74.

Alternatively, conduit section 31 itself may be expandable, in that the conduit section may comprise a moveable wall that expands outward as urine flows into the conduit section. For example, the moveable wall may expand outward from its default (or “relaxed”) position as urine flows into the conduit section, and then collapse back to its default position as urine is pumped out. Alternatively, the moveable wall may collapse inward from its default position as urine is pumped out, and then expand back to its default position as urine flows in. In such embodiments, the sensor may be configured to communicate, to the controller, a signal that varies as a function of the amount of urine in the conduit section.

In other embodiments, conduit 371 comprises a reservoir that does not expand, and sensor 50 senses the amount of urine in the reservoir.

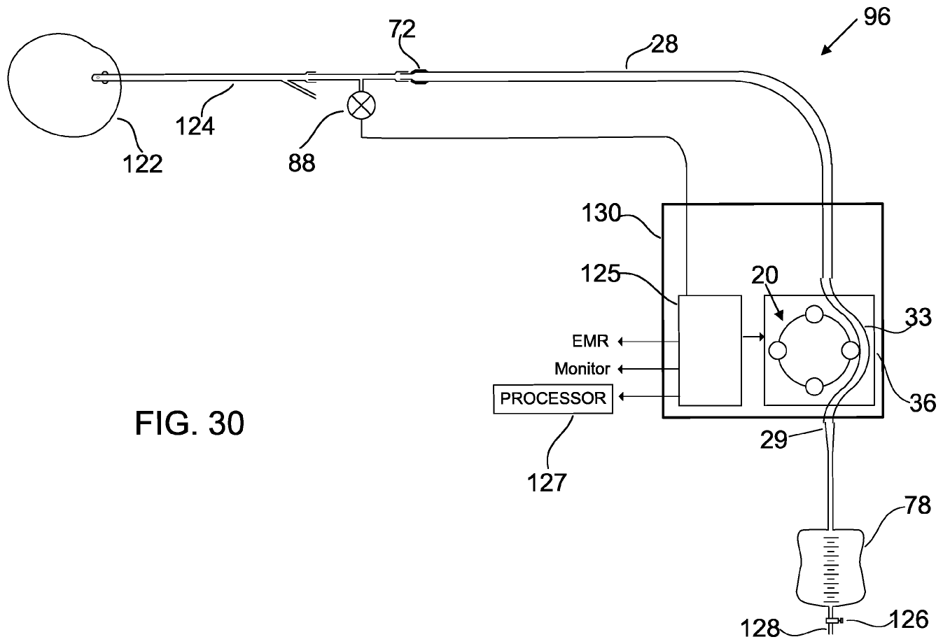

In some embodiments, as described below with reference to FIG. 30, kit 370 comprises a pressure sensor configured to sense the pressure at the outlet of the urinary catheter and to communicate a signal indicating the pressure to the controller. Alternatively, the pressure sensor may be connected to the catheter connector or to any other portion of conduit 371 near the catheter or downstream therefrom. In such embodiments, though kit 370 may comprise reservoir 40 and sensor 50, the kit need not necessarily comprise these components, given that the controller may control the pumping of urine responsively to the signal from the pressure sensor. (In effect, the bladder functions as a reservoir, in that the pressure at the outlet of the urinary catheter increases as a function of the amount of urine in the bladder.)

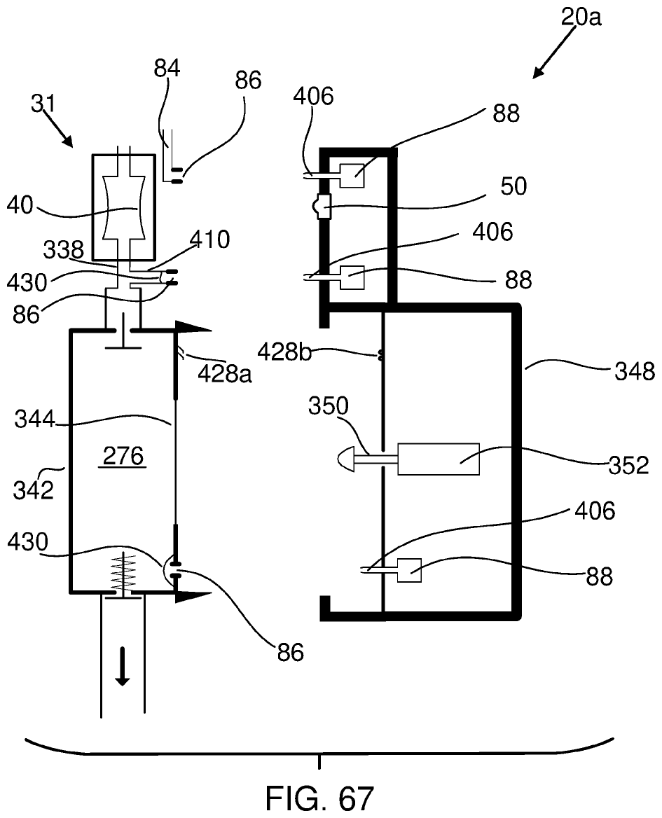

Alternatively, for embodiments in which the amount of urine in the conduit section increases as urine is produced (e.g., for embodiments in which the conduit section comprises a chamber comprising a moveable wall that expands outward as urine flows into the chamber), kit 370 may comprise a sensor (e.g., a pressure sensor) configured to communicate a signal that varies as a function of the amount of urine in the conduit section. In such embodiments, as well, conduit 371 need not necessarily comprise reservoir 40 or sensor 50. (In effect, the conduit section functions as a reservoir.) Such embodiments are described below with reference to FIG. 67, for example.

Alternatively, the (non-disposable) urine-pumping device, rather than kit 370, may comprise a pressure sensor. In such embodiments, kit 370 may further comprise a connection port coupled to tube 28 or to conduit section 31 and configured to couple to a tube containing a fluid, such that the pressure of the fluid varies in response to the pressure in the tube or conduit section. (Optionally, kit 370 may further comprise the fluid-filled tube.) The pressure sensor belonging to the urine-pumping device may thus sense the fluid pressure, and the controller may control the pumping of urine responsively to the fluid pressure. Such embodiments are described below with reference to FIGS. 58, 61 and 67, for example.

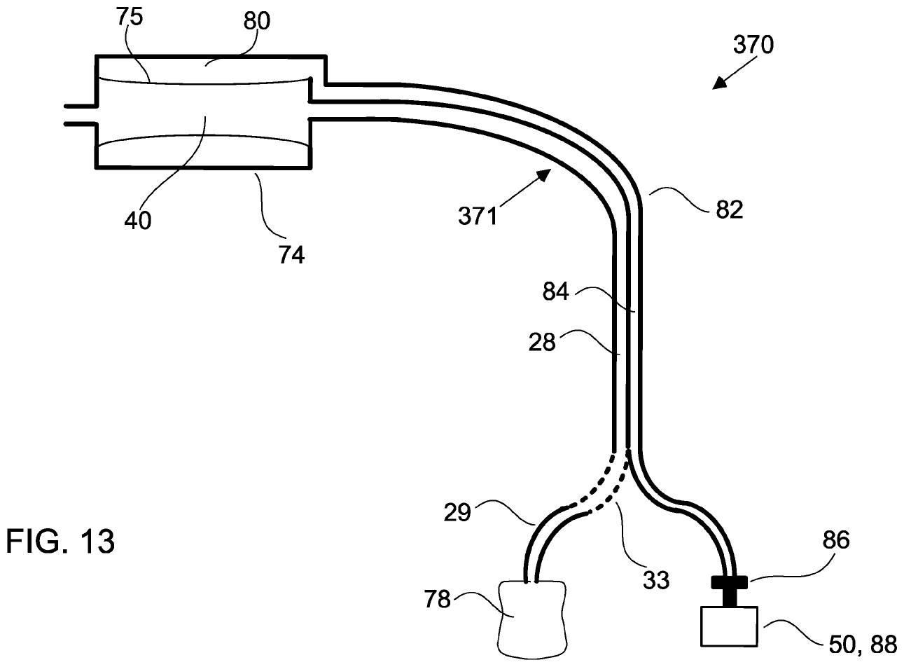

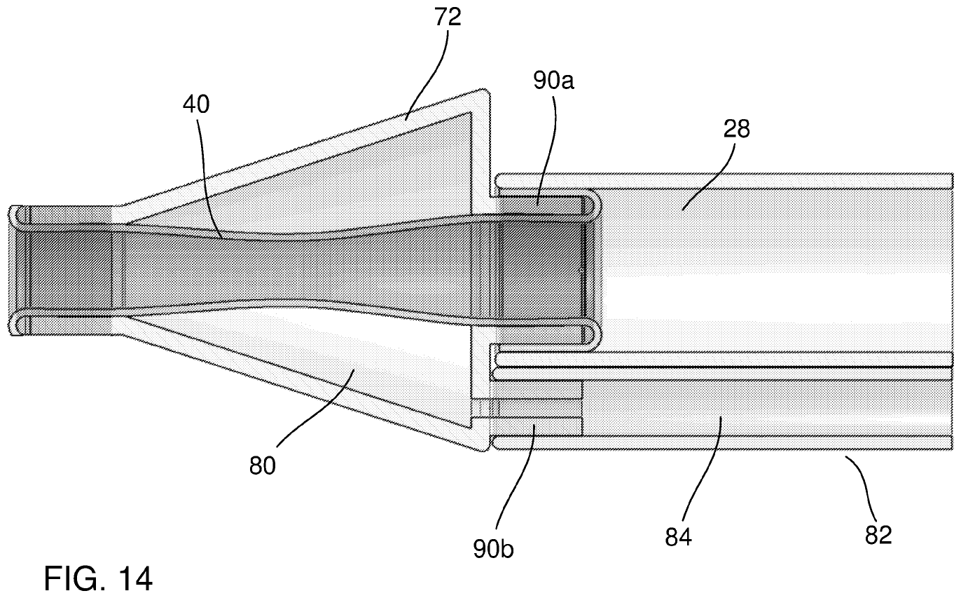

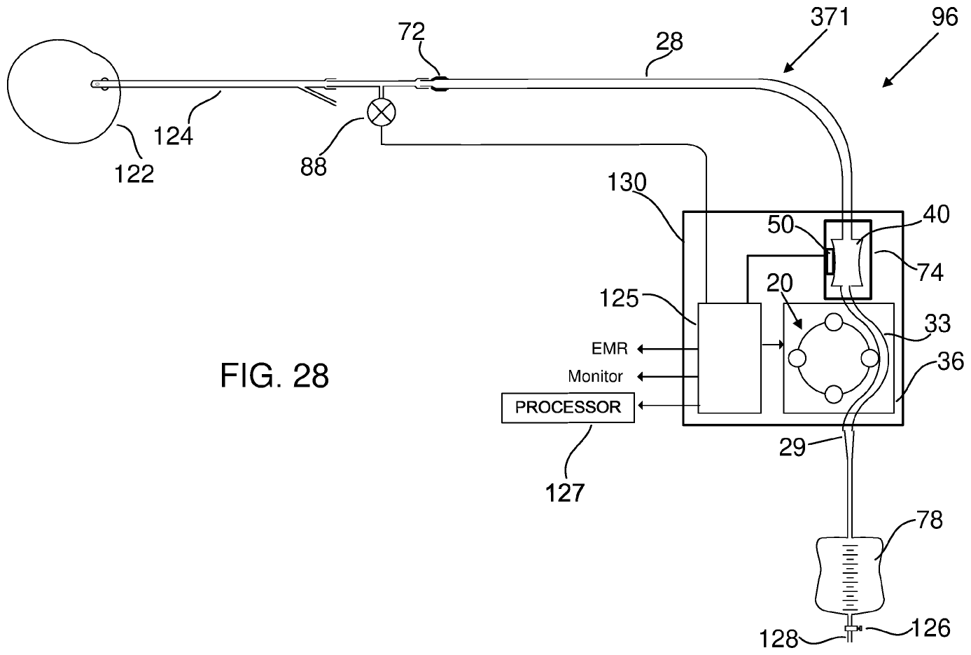

In some embodiments, kit 370 further comprises a catheter connector 72, which is configured to couple, at its upstream end, to the urinary catheter (optionally via another connector as shown in FIG. 30), so as to establish fluid communication between the urine lumen of the catheter and the lumen of connector 72. The downstream end of catheter connector 72 may couple to reservoir 40 such that the lumen of the catheter connector is in fluid communication with the reservoir. Alternatively, for embodiments in which the reservoir is omitted or is integral with the catheter connector (e.g., per FIG. 14), the downstream end of catheter connector 72 may couple to tube 28 (as in FIG. 28) or to conduit section 31 (as in FIGS. 50-51 and 65).

In some embodiments, catheter connector 72 is shaped to define a sampling port 372, via which a sample of urine may be extracted from the lumen of the catheter connector. Alternatively, sampling port 372 may be located in tube 28 or at any other suitable location along the conduit.

In other embodiments, catheter connector 72 is omitted, and the urinary catheter is coupled directly to reservoir 40, to tube 28, or to conduit section 31.

In some embodiments, conduit 371 further comprises the urinary catheter, which may optionally comprise a temperature sensor configured to sense the temperature of the urine.

As described above, controller 125 is configured to control the force-applying elements such that the force-applying elements apply pressure to the conduit, thereby squeezing urine downstream from the conduit. The controller is further configured to calculate the volume of urine that was squeezed, based on the controlling of the force-applying elements. For example, a rotary peristaltic pump 20 may be configured to pump a volume of urine that is known for any given rotation or fractional rotation, such that the controller may calculate the volume of pumped urine based on the number of rotations or fractional rotations executed by the pump and the respective volumes pumped during the rotations or fractional rotations. Further details regarding such calculation are described below in the section entitled “Calculating the pumped volume.”

In general, for embodiments in which the force-applying elements belong to the urine-pumping device, conduit 371 (in particular, conduit section 31) and the force-applying elements may be reversibly coupled to one another via any suitable mechanism.

For example, urine-pumping device 129 may comprise a case coupled to the force-applying elements, and the force-applying elements may reversibly couple to the conduit by virtue of the case reversibly coupling to the conduit. An example of a case that may be reversibly coupled to the conduit is a control unit 130, which contains controller 125.

For example, the conduit (or at least conduit section 31) may be at least partly contained in a cartridge 374, the case may be shaped to define a slot 376, and the case may reversibly couple to the conduit via insertion of the cartridge into the slot. For example, for embodiments in which pump 20 comprises a rotor and conduit section 31 comprises peristaltic pump tube 33, cartridge 374 may be inserted into slot 376 such that the rotor contacts the peristaltic pump tube. To uncouple the conduit from the case (e.g., when transferring urine-pumping device 129 to another subject), the cartridge may be simply slid from the slot, optionally following the execution of a release mechanism.

As another example, as further described below with reference to FIGS. 63A-B and 64A-B, the conduit may be coupled to one or more latches, and the case may reversibly couple to the conduit by virtue of the latches latching onto the case. Alternatively, the case may comprise one or more latches configured to latch onto a housing of the conduit, thereby reversibly coupling the case to the conduit.

In some embodiments, the control unit comprises a start/stop button 298. Alternatively or additionally, the control unit may comprise an insert/eject button 300 that is pressed when coupling the conduit to the case and prior to uncoupling the conduit from the case. (The pressing of button 300 prior to uncoupling the cartridge may execute the aforementioned release mechanism.)

In some embodiments, control unit 130 further comprises a display (or “monitor”) 378, typically comprising a touch screen. In such embodiments, controller 125 is configured to display relevant output, and/or receive relevant input, via display 378. Alternatively or additionally, the controller may be configured to display relevant output, and/or receive relevant input, via another peripheral device (such as a patient monitor, a display, a keyboard, or a mouse) or another computer connected wiredly or wirelessly to the control unit.

In some embodiments, control unit 130 comprises a coupling mechanism 380 comprising, for example, one or more clamps or hooks. Using coupling mechanism 380, the control unit may be coupled to the railing of a subject's bed or to any other suitable structure.

As described in detail below, many variations of system 96 are within the scope of the present invention. For example, a pressure sensor, reservoir, and/or pressure regulator may be connected to or integrated into catheter connector 72, housing 74, or the catheter itself. (The reservoir may comprise or function as a pressure safety valve.) Alternatively or additionally, kit 370 may comprise, at the downstream end of tube 29, a bag connector configured to connect to the urine-collection bag. Alternatively or additionally, the kit may comprise the urine-collection bag. Optionally, the bag may comprise a draining valve for draining urine therefrom. Alternatively or additionally, the bag may comprise a one-way valve at the bag inlet. Alternatively or additionally, the kit may comprise a data-storage medium (e.g., a QR code and/or memory) for storing data including, for example, a subject ID number, a serial number, a manufacturing lot number, an expiration date, kit calibration parameters, security codes, a kit type, or measured parameters associated with the subject. Alternatively or additionally, the kit may comprise a suction-relief tube for increasing the upstream pressure. Alternatively or additionally, cartridge 374 may comprise additional parts that interface with pump 20, such as the clamp described below.

In general, controller 125, in addition to each of the other processors described herein, may be embodied as a single processor, or as a cooperatively networked or clustered set of processors. The functionality of controller 125, and/or the functionality of any of the other processors described herein, may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, controller 125, and/or any of the other processors described herein, may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the controller or processor in electronic form, over a network, for example. Alternatively or additionally, the program code and/or data may be provided and/or stored on non-transitory tangible media, such as magnetic, optical, or electronic memory. Such program code and/or data, when provided to the controller or processor, produce a machine or special-purpose computer, configured to perform the tasks described herein.

Reference is now made to FIG. 65, which is a schematic illustration of system 96, in accordance with some embodiments of the present invention.

In some embodiments, system 96 comprises a case 336, which is coupled to at least some force-applying elements (e.g., a pump rotor) and is separate from control unit 130. Typically, case 336 couples to conduit section 31 upstream from the control unit.

In such embodiments, case 336 may be reversibly or non-reversibly connected to control unit 130 (and hence, to the controller contained therein) by any suitable connection medium. For example, for embodiments in which the pump actuator is coupled to case 336, the controller may be connected to the actuator via electrical wiring 366, and may control the actuator by controlling the voltage, current, duty cycle, and/or frequency of electrical power supplied over wiring 366. Alternatively, the controller may be connected to the actuator via an optical fiber, and may control the actuator by controlling the intensity and/or wavelength of light supplied through the fiber. Alternatively, the controller may be connected to the actuator via a wire or string inside a tube, and may control the actuator by controlling the (linear or radial) mechanical force supplied to the wire or string. Similarly, for embodiments in which the actuator is inside control unit 130, the actuator may actuate the upstream pump components via a wire or string inside a tube.

Alternatively or additionally, for embodiments comprising pneumatic or hydraulic sensing and/or pump actuation, the control unit may be connected to the case via one or more tubes 368. To control the pneumatic or hydraulic actuation, the controller may control the pressure in the appropriate tube 368, e.g., by controlling an air compressor and/or one or more valves. For pneumatic or hydraulic sensing, the control unit may comprise a pressure sensor configured to sense the pressure in the appropriate tube 368.

Similarly, for embodiments in which a sensor is coupled to case 336, signals from the sensor may be communicated to the control unit through electrical wiring 366, tubes 368, or any other connection medium. The sensor may comprise, for example, an optical sensor configured to detect the deflection of a reservoir wall or a diaphragm 430 (FIG. 67) toward or away from the optical sensor.

In general, the aforementioned connection media may be permanently connected to control unit 130, or reversibly connected via matching connectors.

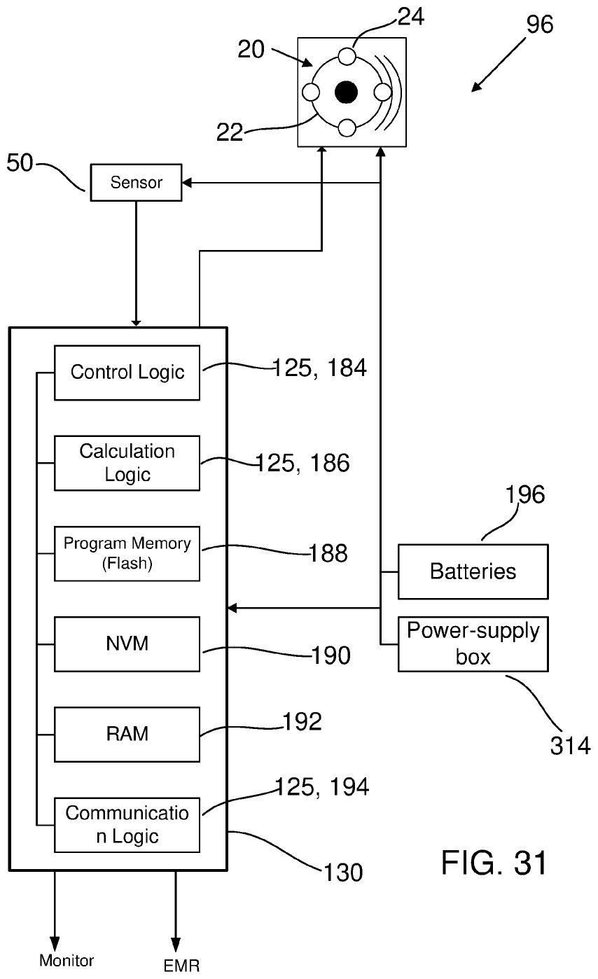

Reference is now made to FIG. 31, which shows a block diagram of some components of system 96, in accordance with some embodiments of the present invention.

In some embodiments, as further described below with reference to FIG. 1A, peristaltic pump 20 comprises a rotor 22, which comprises one or more rollers 24. In some embodiments, the rotation of rotor 22 is quantized into fractional rotations referred to herein as “strokes.” Typically, the number of strokes in a full rotation is equal to the number of rollers 24; for example, with four rollers, each rotation includes four strokes. (More generally, for any type of pump, the term “stroke” is used herein to refer to a single pumping action performed by the pump.)

Typically, controller 125 executes a control-logic module 184, which controls pump 20 in response to output from sensor 50, which monitors a reservoir, and/or one or more other sensors (e.g., a pressure sensor), as further described below in the section entitled “Reservoirs and sensor for pump control.” As the pump is operated, control-logic module 184 communicates data relating to the activity of the pump (e.g., the time of each stroke and/or the time between successive strokes) to a calculation-logic module 186, which is also executed by the controller. Based on these data, calculation-logic module 186 calculates the pumped volume of urine, and hence the rate of urine output and/or production, as a function of time. In some embodiments, as further described below in the section entitled “Noise filtering and display,” the calculation-logic module also filters out any noise that may affect the calculation; such noise may be due to mechanical or biological factors.

In addition to the pump-activity data described above, the calculation-logic module may calculate the volume of each stroke based on any other relevant data such as an elapsed number of previous strokes, an elapsed amount of time (e.g., an elapsed amount of time from the previous stroke or from the start of operation), the ambient temperature, the urine temperature, the pump inlet pressure, the pump outlet pressure, calibration parameters, or the pump speed.

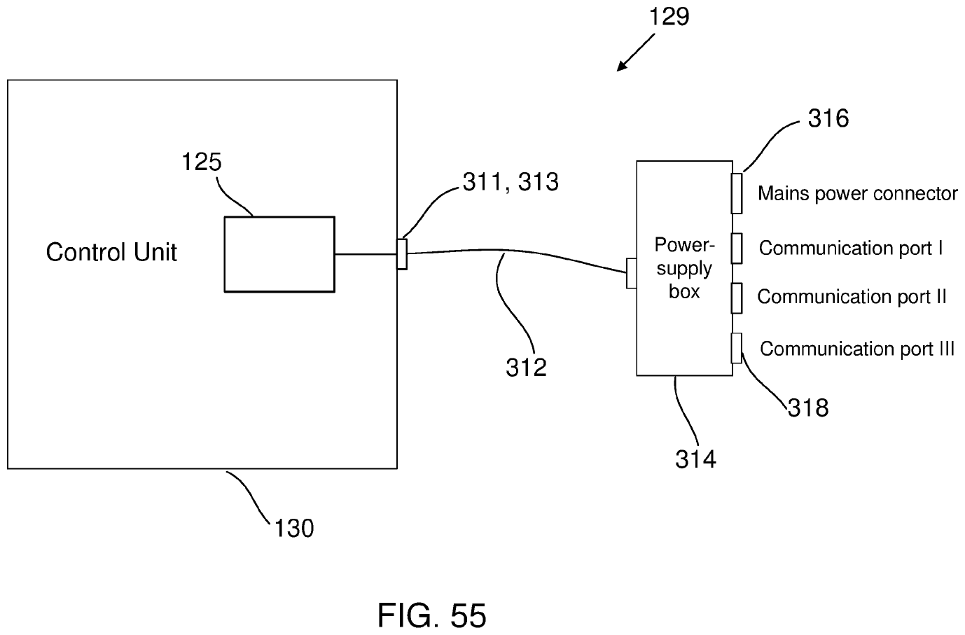

Typically, control unit 130 is connected wiredly or wirelessly to a patient monitor, a dedicated display (e.g., display 378 (FIG. 66B)), a computer network, a gateway, a nurse station monitor, a cellphone, a tablet, an EMR, another computer, and/or another device (e.g., an intravenous pump). (Typically, any wired connections pass through a cable, as described below with reference to FIG. 55.) Controller 125 may further execute a communication-logic module 194, which communicates relevant output, such as calculated parameters relating to the production of urine, to any of these entities. Optionally, communication-logic module 194 may also receive relevant input, such as subject data (e.g., an ID or weight of the subject) or alert thresholds, from any of these entities.

Typically, control unit 130 comprises a program memory (e.g., flash memory) 188, which may store software code for the aforementioned modules. In some embodiments, the control unit further comprises a non-volatile memory (NVM) 190, which may store data such as calibration parameters, measurement data, subject data, or alert thresholds. Alternatively or additionally, the control unit may comprise a random access memory (RAM) 192 for executing the aforementioned modules.

In some embodiments, system 96 further comprises a power-supply box 314, which is configured to power components of the system such as the controller, the pump (in particular, the pump actuator), and sensor 50, as further described below with reference to FIG. 55. System 96 may further comprise one or more batteries 196, which are configured to power the aforementioned components when the control unit is disconnected from the power-supply box or when the power-supply box is disconnected from the mains. Batteries 196 may be rechargeable, and may be charged by power-supply box 314. Alternatively, the system may comprise batteries 196 without power-supply box 314.

Reference is now made to FIG. 55, which is a schematic illustration of control unit 130 connected to power-supply box 314, in accordance with some embodiments of the present invention.

In some embodiments, control unit 130 is connected to a single cable 312 used for both power and communication. In such embodiments, even if the control unit is coupled to the subject's bed, it is relatively easy to move the bed, given that only a single cable needs unplugging.

In such embodiments, control unit 130 comprises an electrical interface 311 connected to controller 125 and configured to couple to cable 312 such that the controller is powered via cable 312. (One or more other components of urine-pumping device 129, such as a pump actuator and/or a sensor, may also be powered via the cable.) The control unit further comprises a communication interface 313, such as an Ethernet networking interface, connected to the controller and configured to couple to the cable. (Optionally, as indicated in FIG. 55, electrical interface 311 and communication interface 313 may be contained in a single unit such as a Universal Serial Bus (USB) Type-C connector.)

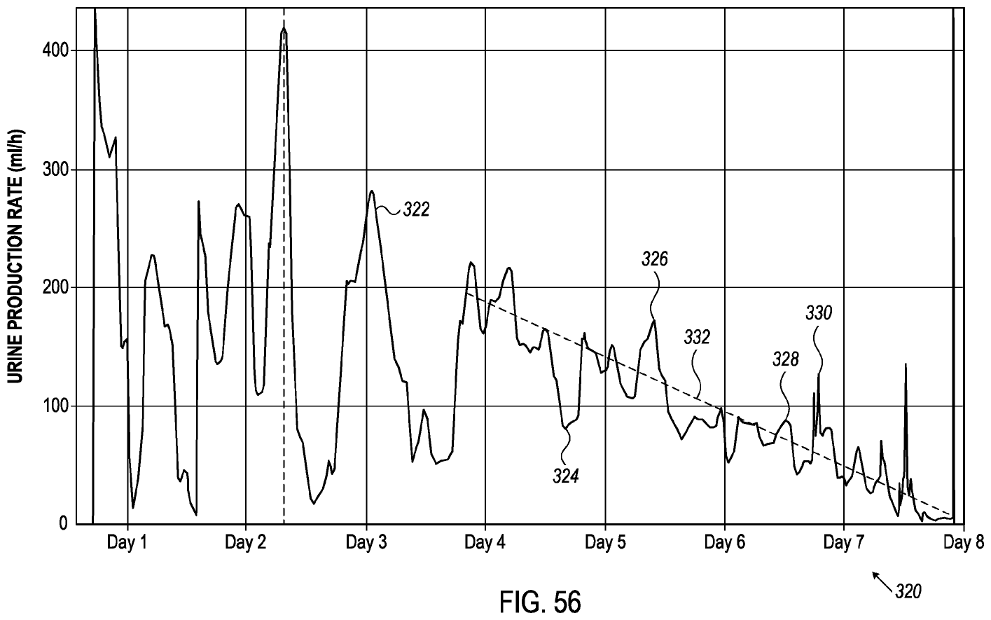

The controller is configured to exchange any relevant communication via the communication interface and the cable. For example, via the communication interface and the cable, the controller may output a calculated volume of pumped urine, a parameter derived from the aforementioned volume (e.g., a rate of urine production or a representative rate of change in this rate, as described below with reference to FIG. 56), or an intra-abdominal pressure (IAP). Alternatively or additionally, via the communication interface and the cable, the controller may receive input such as an operation command (e.g., a start or stop command), a subject ID or weight, or an alert threshold.

Typically, urine-pumping device 129 further comprises power-supply box 314, which facilitates the exchange of power and communication. Advantageously, given that the power-supply box is stationary, the EMR may locate the subject based on communication from the power-supply box.

Power-supply box 314 comprises a mains power connector 316 for connecting to an alternating current (AC) main power supply of the hospital. The power-supply box further comprises one or more communication ports 318, each of which may be connected to a patient monitor, a hospital network, an EMR, or any other suitable device or system. The power-supply box may further comprise one or more electronic components associated with the communication lines such as a surge protector, an electromagnetic interference (EMI) filter, a radio frequency interference (RFI) filter, or an isolator. Alternatively or additionally, the power-supply box may comprise circuitry for intermediating communication between the controller and the devices or systems to which the communication ports are connected.

In some embodiments, the power-supply box further comprises a non-volatile memory configured to store information relating to the subject, such as the subject's ID and/or physiological parameters. An advantage of storing these data in the power-supply box is that even if a control unit is replaced, the data may be restored from the power-supply box.

In some embodiments, to further facilitate moving the subject's bed, the control unit comprises a breakaway connector configured to mate with a breakaway connector at the end of cable 312. The breakaway connectors are configured to separate from one another when a force pulling the breakaway connectors apart from one another exceeds the force that holds the two connectors together. For example, the breakaway connectors may be coupled to one another by magnets, by a spring, by friction between the walls of the connectors, or by a vacuum force.

In alternate embodiments, instead of an external power-supply box, the control unit comprises an integrated power supply, and the components of power-supply box 314 detailed above are integrated into the control unit.

Various aspects of system 96 are hereby described in further detail.

### Pumps

I. Pumps with Pressing Elements

### (a) Peristaltic Pumps

Reference is now made to FIG. 1A, which is a schematic illustration of peristaltic pump 20, in accordance with some embodiments of the present invention.

As described above with reference to FIGS. 66A-B, in some embodiments, the force-applying elements that apply force to the conduit, thereby forcing urine downstream, comprise a pressing element and an actuator, which is controlled by controller 125.

For example, the urine-pumping device may comprise peristaltic pump 20. In some embodiments, the peristaltic pump comprises a rotor 22 comprising a plurality of (e.g., four) rollers 24. Rotor 22 is configured to rotate, in response to torque applied by an actuator, while pressing against peristaltic pump tube 33 (FIG. 66A), thereby displacing urine from the tube in a direction corresponding to the rotation direction of the rotor. The actuator may comprise a direct current (DC) motor, a stepper motor, a brushless motor, a pneumatic or hydraulic motor, or a pneumatic or hydraulic piston.

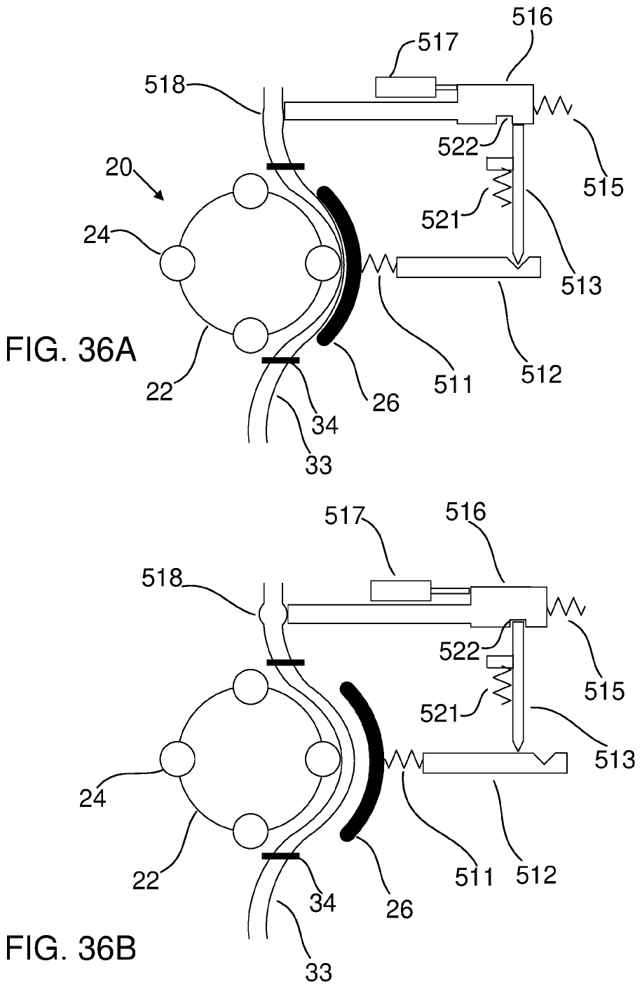

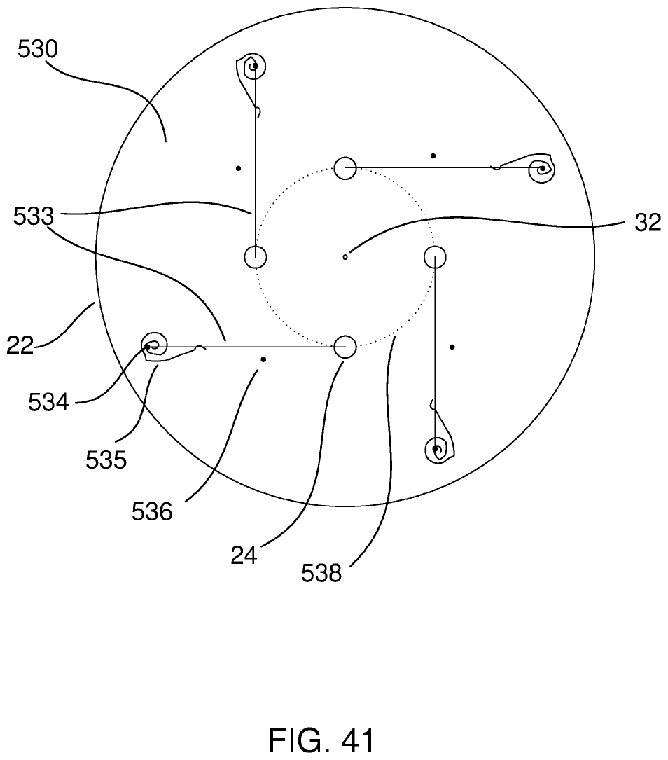

Typically, pump 20 further comprises a clamp 26, which is configured to clamp the peristaltic pump tube onto rotor 22 so as to facilitate this operation. In some embodiments, as described below with reference to FIGS. 36A-B and 39A-C, clamp 26 is coupled to a spring, which presses the clamp onto the rotor. Alternatively or additionally, one or more springs may be coupled to the rotor, as described below with reference to FIGS. 41-46.

Typically, rotor 22 is mounted onto, and rotates about, an axle 32, which is coupled to a pump base 36. In some embodiments, pump base 36 is shaped to define a pair of sockets 30, whose function is described immediately below. Pump base 36 may be contained, for example, within control unit 130 (FIG. 66B).

Reference is now further made to FIG. 1B, which is a schematic illustration of peristaltic pump tube 33 in accordance with some embodiments of the present invention, and to FIG. 1C, which is a schematic illustration of pump 20 mechanically coupled to the peristaltic pump tube in accordance with some embodiments of the present invention.

In some embodiments, a pair of tube anchors 34, which may be U-shaped, anchor the peristaltic pump tube to base 36 both upstream and downstream from clamp 26, e.g., by virtue of being lodged into sockets 30 such that the tube is held against the base by the tube anchors. Advantageously, the tube anchors inhibit the portion of tube 33 between the tube anchors from being stretched or compressed, thereby facilitating a more precise calculation of the volume of urine displaced from the tube by pump 20. In such embodiments, cartridge 374 (FIG. 66A) may comprise tube anchors 34 such that, as the cartridge is inserted into the control unit (which contains the pump base), the tube anchors enter the sockets.

In other embodiments, tube anchors 34 (permanently) anchor tube 33 to the cartridge, and sockets 30 are omitted.

FIG. 1C shows clamp 26 pressing tube 33 against rotor 22, thereby mechanically coupling the tube to the rotor. A silhouette 38 of the clamp marks an initial position of the clamp prior to the mechanical coupling.

As described below in the section entitled “Reservoirs and sensors for pump control,” peristaltic pump 20 may be controlled responsively to various types of sensor signals.

### (b) Reciprocating Pumps

Reference is now made to FIGS. 63A-B, which are schematic illustrations of a reciprocating pump 20a together with conduit section 31, in accordance with some embodiments of the present invention.

In some embodiments, urine-pumping device 129 (FIG. 66B) comprises reciprocating pump 20a, which comprises a plunger 350 (or another type of pressing element) and actuator 352. In response to control signals from the controller, the actuator advances and retracts plunger 350 such that the plunger repeatedly presses against the conduit section, thereby displacing urine from the conduit section. Typically, the plunger and actuator are coupled to a case 348 that may be coupled to control unit 130 (FIG. 66B), e.g., within the control unit. (As noted above with reference to FIG. 66B, the control unit itself may also be referred to as a “case.”)

In such embodiments, typically, conduit section 31 comprises a chamber housing 342 that encloses a pump chamber 276. The front wall of chamber housing 342, which faces pump 20a, is shaped to define an opening that is filled by a moveable wall 343. Conduit section 31 and pump 20a are configured to couple to one another such that, as the plunger is advanced, the plunger presses against moveable wall 343.

Conduit section 31 further comprises an inlet port 338, which is separated from pump chamber 276 by an inlet valve 274. Inlet port 338 is configured to couple to the urinary catheter (optionally via catheter connector 72 and/or tube 28 (FIG. 66A)) such that, as urine is produced by the subject's kidneys, the urine flows into the pump chamber via inlet port 338 and inlet valve 274.

Conduit section 31 further comprises an outlet port 340, which is separated from pump chamber 276 by an outlet valve 284. Outlet valve 284 may be held closed by a biasing spring 286. As the plunger presses against moveable wall 343, the moveable wall moves inward, such that the volume of the pump chamber is reduced and urine is forced through outlet valve 284 and into outlet port 340. Outlet port 340 is coupled to exit tube 29 (FIG. 66A) or directly to the urine-collection bag.

Conduit section 31 and pump 20a may couple to one another using any suitable mechanism. For example, the conduit section may be contained in cartridge 374, which may be inserted into control unit 130 as described above with reference to FIG. 66B. Alternatively, for example, housing 342 may be coupled to one or more latches 346 that latch onto case 348. For example, the side walls of case 348 may comprise respective frontal protrusions 354 that protrude inward, toward the middle of the frontal opening of case 348 that faces conduit section 31. Latches 346 may be inserted through the frontal opening, between frontal protrusions 354, such that, upon passing the front protrusions, the latches snap outward (i.e., sideward) and latch onto the frontal protrusions, as shown in FIG. 63B. Alternatively, frontal protrusions 354 may face outward, and the latches may snap inward and latch onto the frontal protrusions.

In some embodiments, moveable wall 343 comprises a diaphragm 344. During each pump stroke, actuator 352 advances plunger 350 such that the plunger pushes diaphragm 344 from its relaxed position 356 into chamber 276, thereby forcing urine through outlet valve 284 and outlet port 340. In some embodiments, the advancement of the plunger is performed at the start of the stroke; following the advancement, the plunger is retracted, such that the diaphragm returns to relaxed position 356 and urine is drawn into the chamber from inlet port 338 through inlet valve 274. In other embodiments, during each pump stroke, the actuator first retracts the plunger, thereby drawing urine into the chamber, and then advances the plunger, thereby forcing urine from the chamber.

Actuator 352 may comprise an electrical actuator, a pneumatic actuator, or a hydraulic actuator. For example, the actuator may comprise an electrical solenoid, a linear motor, a motor with a leadscrew, a motor with a ball screw, a motor with a roller screw, or a motor with a traveling nut. Alternatively, the actuator may comprise a DC motor, a stepper motor, a brushless motor, a pneumatic or hydraulic motor, or a pneumatic or hydraulic piston, any of which may be coupled to a camshaft for linear actuation. The actuator is connected to the controller by control lines (not shown).

Reference is now made to FIGS. 64A-B, which are schematic illustrations of reciprocating pump 20a together with conduit section 31, in accordance with other embodiments of the present invention.

In some embodiments, housing 342 comprises a cylinder 360, which opens into (and is thus in fluid communication with) chamber 276. Moveable wall 343 comprises a piston 358 disposed within cylinder 360. During each pump stroke, actuator 352 advances plunger 350 such that the plunger presses against piston 358, thereby pushing urine through outlet valve 284. The plunger may be advanced at the start or end of each stroke, as described above for FIGS. 63A-B.

In some embodiments, piston 358 is shaped to define a socket 362, which is configured to fittingly receive a plunger head 364 of plunger 350. Thus, as the plunger is retracted, the plunger pulls the piston along.

As described below in the section entitled “Reservoirs and sensors for pump control,” reciprocating pump 20a may be controlled responsively to various types of sensor signals.

II. Pumps with Fluid-Filled Tubes for Applying Force

In some embodiments, instead of pressing the conduit with a pressing element, a pumping force is applied to the conduit via a fluid, i.e., a gas or liquid, contained within a tube. As the pressure of the fluid is increased, the fluid moves a moveable wall of the conduit.

In this regard, reference is now made to FIG. 50, which is a schematic illustration of system 96, in accordance with some embodiments of the present invention.

The embodiment of FIG. 50 is similar to that of FIGS. 64A-B in that movement of piston 358 causes urine to flow through pump chamber 276. In FIG. 50, however, piston 358 is moved by pneumatic or hydraulic force. In particular, cylinder 360 terminates at a connection port 282, which is configured to couple (or is permanently coupled) to a fluid-filled tube. As actuator 352, which is typically disposed in control unit 130, varies the pressure of the fluid, piston 358 advances toward or withdraws from the opposing wall of the pump chamber.

In some embodiments, conduit section 31 is coupled to three separate tubes. In particular, outlet port 340 is coupled to exit tube 29 (FIG. 66A), connection port 282 is coupled to a fluid-filled tube 291 (FIG. 58), and another connection port 86 is coupled to a (fluid-filled) pressure-conveying tube 406 (FIG. 58), which is used for sensing as further described below in the section entitled “Reservoirs and sensors for pump control.” In such embodiments, fluid-filled tubes 291 and 406 may belong to the (non-disposable) urine-pumping device. Alternatively, fluid-filled tubes 291 and 406 may be permanently coupled to the conduit section, and thus belong to the disposable kit.

In other embodiments, connection port 282 and connection port 86 are coupled to different respective fluid-filled lumens of a single tube, such that conduit section 31 is coupled to two tubes in total. In such embodiments, also, the fluid-filled tube may belong to the urine-pumping device or to the disposable kit.

In yet other embodiments, outlet port 340 and the two connection ports are coupled to different respective lumens of a multi-lumen tube 294 belonging to the disposable kit. In particular, connection port 86 is coupled to a pressure-measurement lumen 288, connection port 282 is coupled to a pressure-application lumen 290, and outlet port 340 is coupled to a urine lumen 292, which leads to urine-collection bag 78.

Actuator 352 may be reversibly coupled to conduit section 31 by reversibly coupling multi-lumen tube 294, or a separate fluid-filled tube, to connection port 282 and/or to the actuator. Any suitable tube connectors known in the art may be used for this coupling.

In some embodiments, the upstream end 262 of inlet port 338 is coupled to catheter connector 72 or directly to urinary catheter 124. In other embodiments, upstream end 262 is coupled to a tube that carries urine from the urinary catheter.

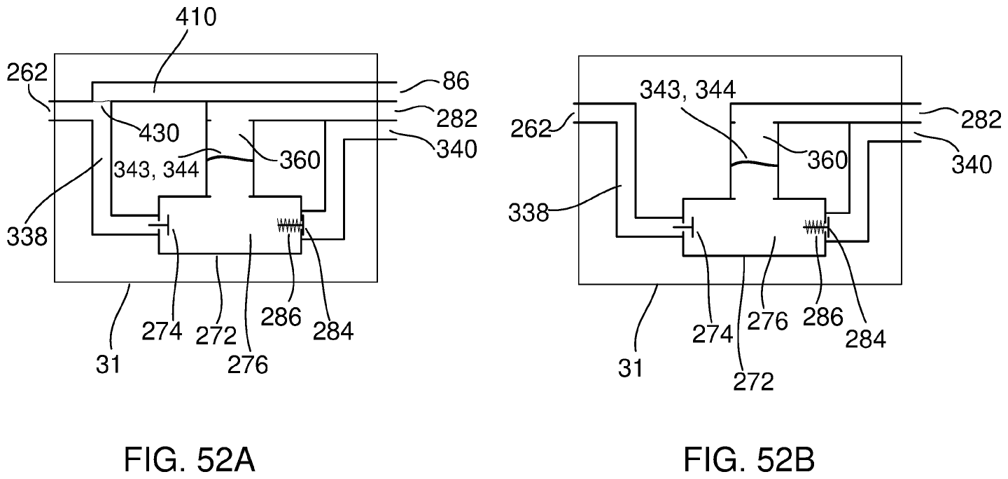

Reference is now made to FIG. 52A, which is a schematic illustration of conduit section 31, in accordance with some embodiments of the present invention.

In FIG. 52A, conduit section 31 is as shown in FIG. 50, except for conduit section 31 comprising diaphragm 344 instead of piston 358. The edge of diaphragm 344 is anchored to the wall of cylinder 360. As the pressure in cylinder 360 is varied, the diaphragm distends toward or away from the opposing wall of the pump chamber.

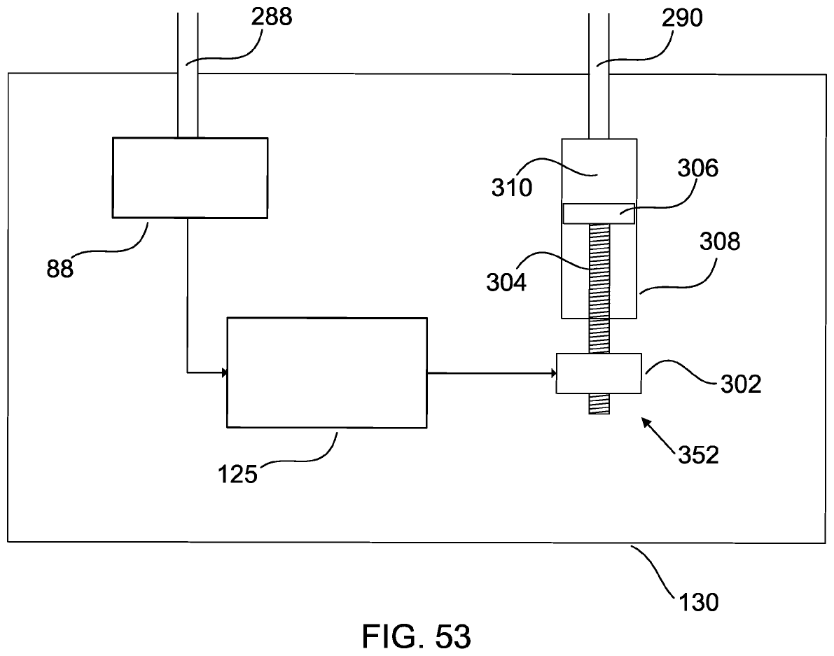

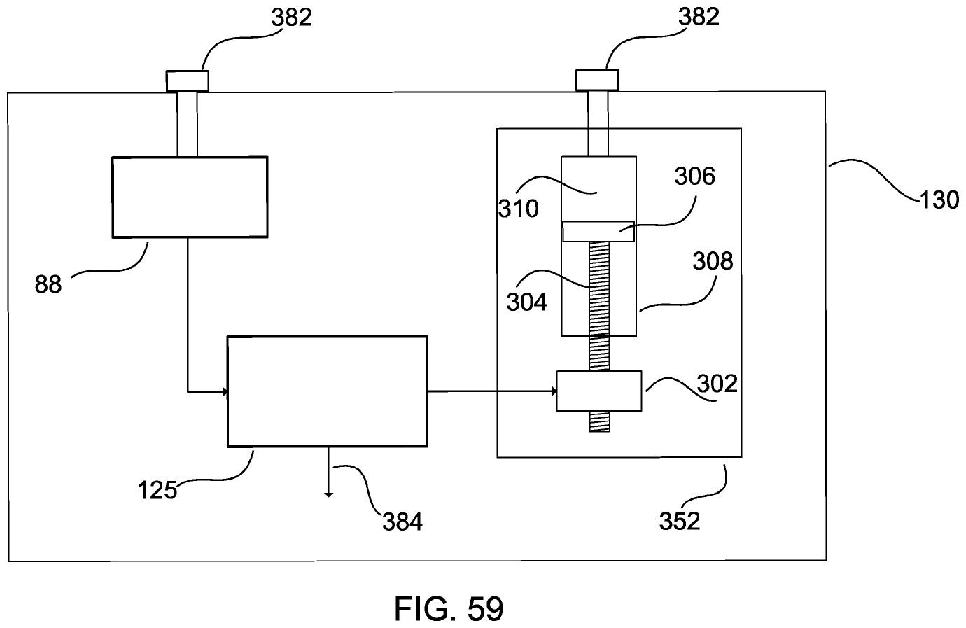

Reference is now made to FIGS. 53 and 59, which are schematic illustrations of control unit 130, in accordance with different respective embodiments of the present invention.

In some embodiments, actuator 352 comprises an actuating component 302, a screw 304 coupled to actuating component 302, and a piston 306 coupled to the end of screw 304. Piston 306 is disposed in a chamber 308, a compartment 310 of which is in fluid communication with pressure-application lumen 290 (or a separate fluid-filled tube). In some embodiments, actuating component 302 comprises a motor such as a DC motor, a brushless motor, or a stepper motor.

To increase the pressure in pressure-application lumen 290 (and hence in cylinder 360 (FIG. 50)), controller 125 drives actuating component 302 to turn the screw such that piston 306 is advanced into compartment 310, thereby compressing the fluid in pressure-application lumen 290. Conversely, to decrease the pressure in the pressure-application lumen, the controller drives the actuating component to turn the screw in the opposite direction. In some embodiments, the controller controls the actuating component responsively to a signal from a pressure sensor that senses the pressure in compartment 310.

In other embodiments, screw 304 is omitted, and actuating component 302 comprises a linearly-actuating solenoid coupled to piston 306 directly.

As further described below in the section entitled “Reservoirs and sensors for pump control,” a pressure sensor 88 may be coupled to pressure-measurement lumen 288 (or to the lumen of a separate fluid-filled tube) and configured to communicate, to the controller, a signal indicating the pressure in the lumen.

In some embodiments, as shown in FIG. 53, the multi-lumen tube is inserted (or separate fluid-filled tubes are inserted) into control unit 130 such that pressure-measurement lumen 288 is in fluid communication with pressure sensor 88 and pressure-application lumen 290 is in fluid communication with chamber 308. In other embodiments, as shown in FIG. 59, the control unit is connected to the tube(s) via connectors 382. (FIG. 59 also shows an output signal 384 from the controller that may indicate, for example, an amount or rate of produced urine.)

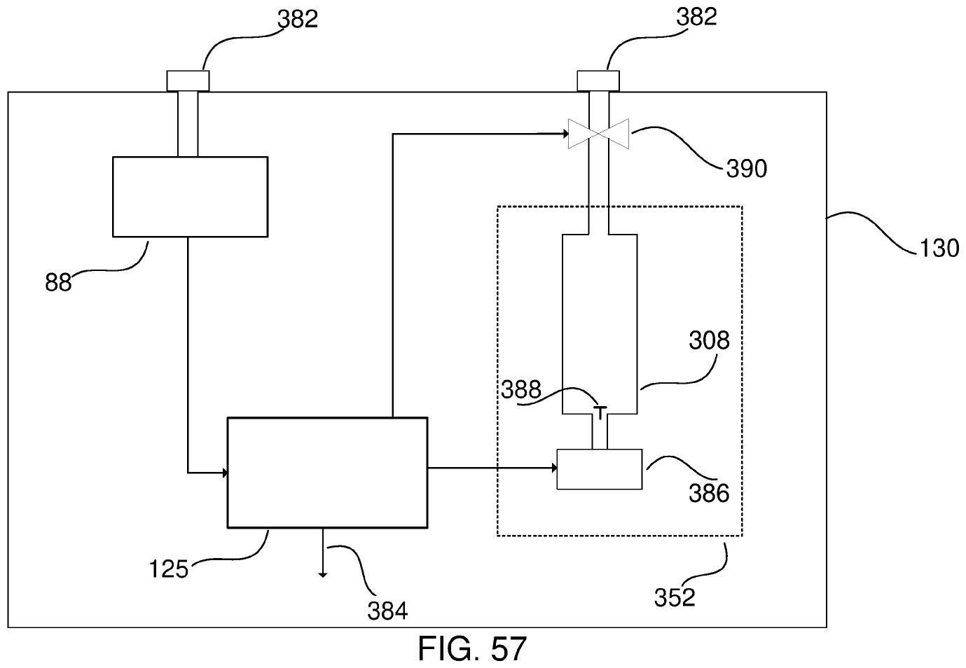

Reference is now made to FIG. 57, which is a schematic illustration of control unit 130, in accordance with other embodiments of the present invention.

In some embodiments, actuator 352 comprises a pump 386, configured to pump a gas (e.g., air) into chamber 308 through an inlet valve 388. An outlet valve 390 regulates the flow of the gas from chamber 308 to pressure-application lumen 290 (FIG. 50) or a separate gas-filled tube, and a third valve (not shown) regulates the release of the gas from the pressure-application lumen. The gas may be released into the surrounding environment or into another chamber similar to chamber 308, which is kept below atmospheric pressure by another pump similar to pump 386.

To increase the pressure in the pressure-application lumen, controller 125 opens outlet valve 390. Conversely, to decrease the pressure in the pressure-application lumen, the controller opens the third valve, such that gas is released from the pressure-application lumen.

Controller 125 also controls pump 386 so as to keep chamber 308 filled with an amount of gas that is sufficient to raise the pressure in the pressure-application lumen to the desired target value whenever the outlet valve is opened. In some embodiments, the controller controls the pump responsively to a signal from a pressure sensor that senses the pressure in chamber 308.

In addition, if the gas is released into another chamber as described above, the controller controls the other pump so as to keep the pressure in the other chamber sufficiently low such that, whenever the third valve is opened, gas is sucked into the other chamber until the desired target value is reached. In some embodiments, the controller controls the other pump responsively to a signal from a pressure sensor that senses the pressure in the other chamber. It is emphasized that the embodiments of FIGS. 53, 57, and 59 are presented herein by way of example only, and that any suitable actuator may be used to apply a pneumatic or hydraulic force to the conduit section. Such an actuator may have two outputs, one for moving the moveable wall in one direction, and the other for moving the moveable wall in the opposite direction. To accommodate the second output, multi-lumen tube 294 (FIG. 51) may be shaped to define an additional fluid-filled lumen.

Reference is now made to FIG. 58, which is a schematic illustration of system 96, in accordance with some embodiments of the present invention.

In some embodiments, the force-applying elements of system 96 comprise a compound actuator comprising two components: actuator 352, which varies the pressure within fluid-filled tube 291 (e.g., as described above with reference to FIG. 57 or FIG. 59), and another actuator 396, which moves a shaft 398 in response to the pressure. Alternatively, a first actuator may apply a linear or radial force to a cable running through a tube, and actuator 396 may move shaft 398 in response to the force.

In such embodiments, system 96 may comprise any suitable positive-displacement pump 394. For example, pump 394 may comprise peristaltic pump 20 (FIG. 66B), in that shaft 398 may be coupled to a rotor or to linear translational elements of the pump. Alternatively, for example, pump 394 may comprise reciprocating pump 20a (FIGS. 63A-B and 64A-B), in that shaft 398 may be coupled to plunger 350 or may comprise plunger 350.

As further shown in FIG. 58, fluid-filled tube 291 and pressure-conveying tube 406 (the function of which is further described below in the following section) may be coupled to connectors 382 via respective connectors 400, such that fluid-filled tube 291 is in fluid communication with the actuator and pressure-conveying tube 406 is in fluid communication with pressure sensor 88 (FIG. 57). Similarly, for embodiments in which a cable-carrying tube substitutes for pressure-conveying tube 406, the cable-carrying tube may be coupled via a connector 400 such that the actuator within the control unit may apply force to the cable.

### Reservoirs and Sensors for Pump Control

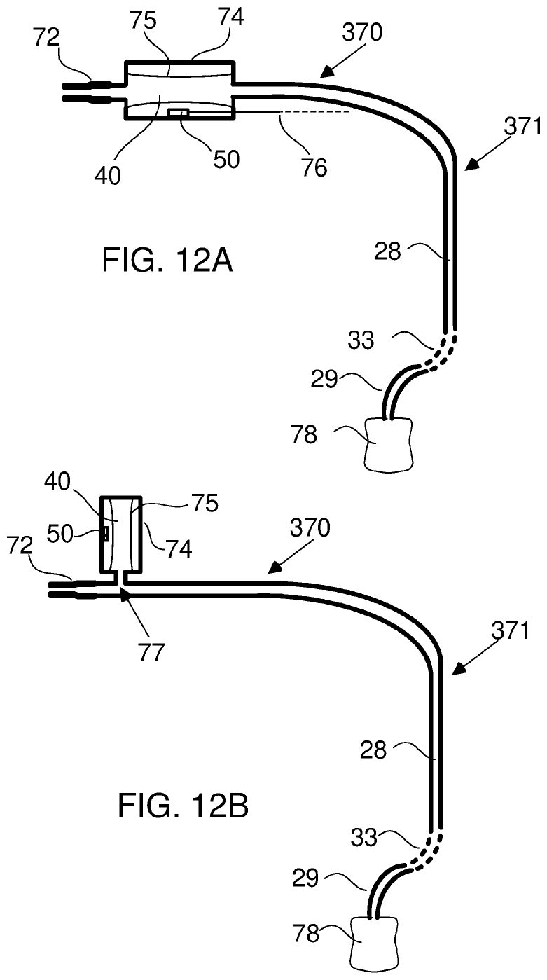

Reference is now made to FIGS. 12A-B, which are schematic illustrations of disposable kit 370, in accordance with some embodiments of the present invention.

As described above with reference to FIG. 66A, conduit 371 of kit 370 may comprise reservoir 40 upstream from the pump. Reservoir 40 may be housed, together with sensor 50, in housing 74 or in catheter connector 72. Typically, reservoir 40 comprises an expandable tube 75 configured to expand as urine flows into tube 75. For example, expandable tube 75 may expand outward from its default (or “relaxed”) state as urine flows into the expandable tube, and then collapse back to its default state as urine is pumped out. Alternatively, the expandable tube may collapse inward from its default state as urine is pumped out, and then expand back to its default state as urine flows in.

Sensor 50 is configured to monitor a parameter indicative of the amount of urine in the reservoir, and to communicate a signal, which indicates the value of the parameter, to the controller. In some embodiments, a signal-carrying element 76, such as a wire or an optical fiber, carries the signal to the controller, e.g., as described below with reference to FIG. 67. (Signal-carrying element 76 may run alongside tube 28 or within a lumen of tube 28.) In other embodiments, sensor 50 comprises a wireless transmitter configured to transmit the signal wirelessly.

As further described above with reference to FIG. 66A and FIGS. 1B-C, conduit 371 may further comprise peristaltic pump tube 33, which is connected to a urine-collection bag 78 via exit tube 29. (As described above with reference to FIG. 1C, peristaltic pump tube 33 is configured to reversibly couple to pump base 36, as indicated by the dashed lines in FIGS. 12A-B.) Alternatively, for example, as described above with reference to FIGS. 63A-B and 64A-B, conduit 371 may comprise pump chamber 276, and outlet port 340 may be connected to urine-collection bag 78 via exit tube 29.

(Similarly, just as reservoir 40 and sensor 50 as shown in FIGS. 12A-B may be combined with any suitable type of conduit section and pump, it is noted that embodiments described below with reference to other figures, such as FIGS. 13 and 67, may be combined with any suitable type of conduit section or pump, notwithstanding the particular conduit sections or pumps shown in these figures.)

In some embodiments, as shown in FIG. 12A, reservoir 40 is coupled (e.g., via housing 74 and/or an intervening tube) at its upstream end to catheter connector 72 (or directly to the urinary catheter) and at its downstream end to tube 28. In other embodiments, as shown in FIG. 12B, reservoir 40 is in fluid communication with tube 28 via a lateral opening 77 in tube 28.

Example embodiments of sensor 50 for FIGS. 12A-B are described below in the section entitled “Example reservoirs and sensors.”

Reference is now made to FIG. 13, which is a schematic illustration of disposable kit 370, in accordance with some embodiments of the present invention.

In some embodiments, a pressure-conveying tube 82, which is shaped to define a fluid-filled capillary lumen 84 (also referred to herein as a “pressure-conveying lumen”), is coupled at its upstream end to housing 74 such that pressure-conveying lumen 84 is in fluid communication with the volume 80 of housing 74 between expandable tube 75 and the walls of the housing. (Alternatively, for embodiments in which catheter connector 72 (FIGS. 12A-B) comprises tube 75, pressure-conveying tube 82 may be coupled directly to the catheter connector.) Volume 80 and lumen 84 are filled with air or another gas.

In such embodiments, sensor 50 (which typically belongs to the urine-pumping device rather than to the disposable kit) comprises a pressure sensor 88. Pressure-conveying tube 82 is coupled at its downstream end to a connection port 86, which is configured to connect to pressure sensor 88 such that the pressure sensor senses the internal pressure within lumen 84 and volume 80. As reservoir 40 expands (or “inflates”) and contracts (or “deflates”), the internal pressure within lumen 84 and volume 80 changes; hence, the internal pressure is indicative of the amount of urine in the reservoir.

In some embodiments, kit 370 comprises a multi-lumen tube shaped to define a pressure-conveying lumen, which functions similarly to the lumen of tube 82, and at least one other lumen, which may carry urine (similarly to the lumen of tube 28) or serve any other function.

Reference is now made to FIG. 14, which is a schematic illustration of catheter connector 72, in accordance with some embodiments of the present invention.

FIG. 14 shows a variation of FIG. 13, in which reservoir 40 is disposed within catheter connector 72. In such embodiments, tube 28 may be coupled to a first port 90a of the catheter connector such that tube 28 is in fluid communication with the internal volume of reservoir 40, and tube 82 may be coupled to a second port 90b of the catheter connector such that capillary lumen 84 is in fluid communication with volume 80. Alternatively, different respective lumens of a multi-lumen tube may couple to first port 90a and second port 90b.

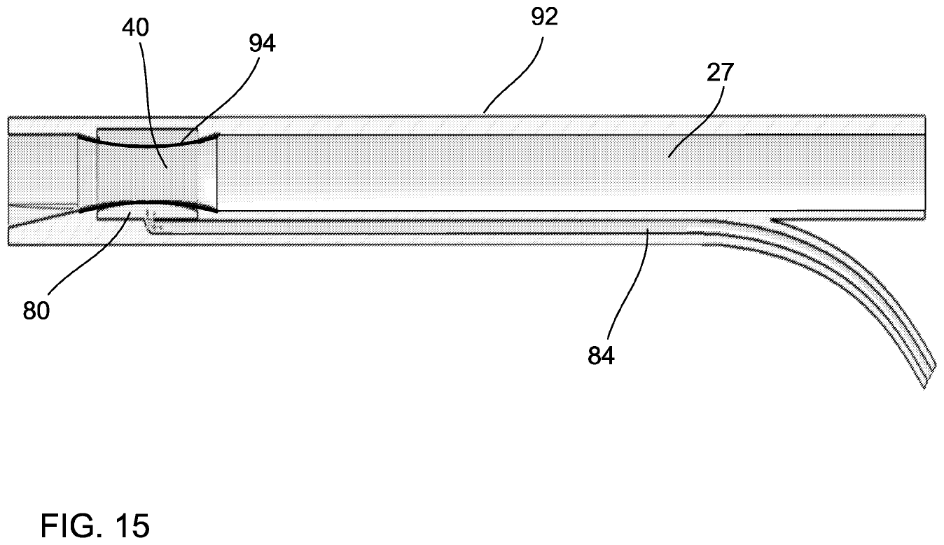

Reference is now made to FIG. 15, which is a schematic illustration of a dual-lumen tube 92, in accordance with some embodiments of the present invention.

In some embodiments, conduit 371 comprises a dual-lumen tube 92 shaped to define two lumens: a wider lumen 27 for carrying urine from reservoir 40, and capillary lumen 84 for pressure conveyance.

In such embodiments, reservoir 40 (comprising expandable tube 75, for example) and volume 80 may be integrated into tube 92. In particular, a wall 94 of the reservoir, which is thinner (and hence more flexible) than (i) the outer wall of tube 92 and (ii) the inner wall separating lumen 84 from lumen 27, may pass through a compartment of tube 92 such that, as wall 94 expands or contracts as a result of changes in pressure inside the reservoir, the pressure within volume 80 of the compartment and lumen 84 changes. (In effect, in such embodiments,

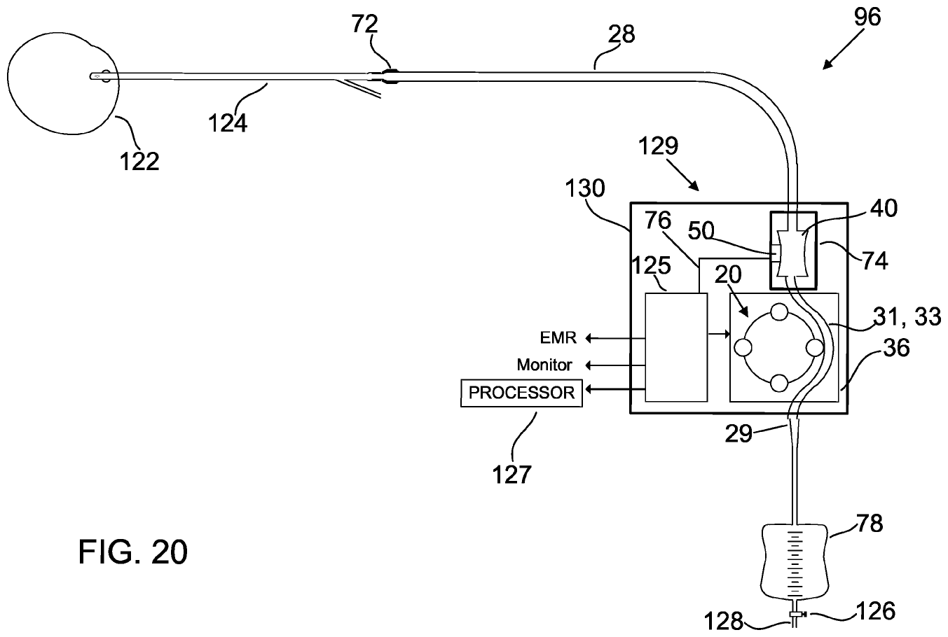

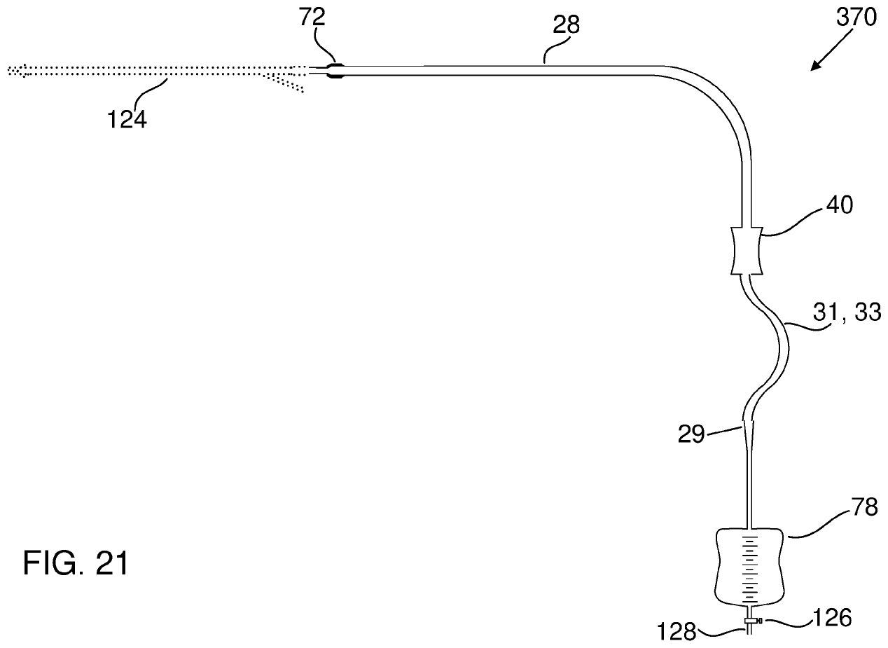

Reference is now made to FIG. 20, which is a schematic illustration of system 96, in accordance with some embodiments of the present invention. Reference is further made to FIG. 21, which is a schematic illustration of disposable kit 370 per FIG. 20, in accordance with some embodiments of the present invention. (In other words, FIG. 21 shows disposable components of FIG. 20.)

In some embodiments, disposable kit 370 is configured to couple to urine-pumping device 129 such that reservoir 40 and conduit section 31 are both disposed within control unit 130. For example, the reservoir may be disposed downstream from tube 28, near conduit section 31, such that the reservoir and conduit section may both be coupled to the control unit. (For example, the reservoir and conduit section may both be disposed in cartridge 374, which may be inserted into slot 376 (FIG. 66B) in the control unit.) The upstream end of tube 28 may be connected (e.g., via catheter connector 72) to the urinary catheter 124 (e.g., the Foley catheter) that catheterizes the bladder 122 of the subject.

In some such embodiments, control unit 130 comprises sensor 50, which is disposed near the position at which the reservoir is coupled to the control unit, e.g., near the slot in the control unit. In other such embodiments, the sensor belongs to kit 370; for example, the sensor may be disposed within cartridge 374 (FIG. 66B), near the reservoir. In either case, as described above with reference to FIGS. 12A-B, controller 125 receives signals from sensor 50 and controls pump 20 responsively thereto.

For embodiments in which the sensor belongs to the kit, signal-carrying element 76 may terminate at a first electrical interface, as described below with reference to FIG. 67. As the kit is coupled to the urine-pumping device (e.g., as the cartridge is inserted into the slot), the first electrical interface may couple with a second electrical interface connected to the controller, such that the signals from the sensor may reach the controller.

Controller 125 may further communicate output, wiredly or wirelessly, to a patient monitor, display 378 (FIG. 66B) and/or an external display (e.g., a display in the doctor's room), an electronic medical record (EMR), and/or another computer processor 127, such as a processor belonging to the doctor's cellphone or tablet. Optionally, all communication from the controller to another device may be delivered via a gateway, a server, or the cloud.

In some embodiments, system 96 further comprises a drainage valve 126 for draining urine-collection bag 78 into a drainage tube 128.

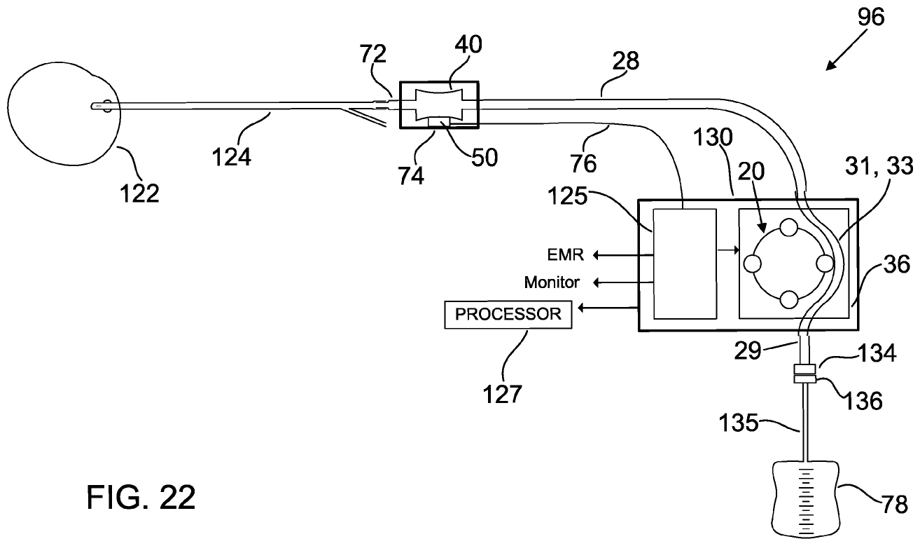

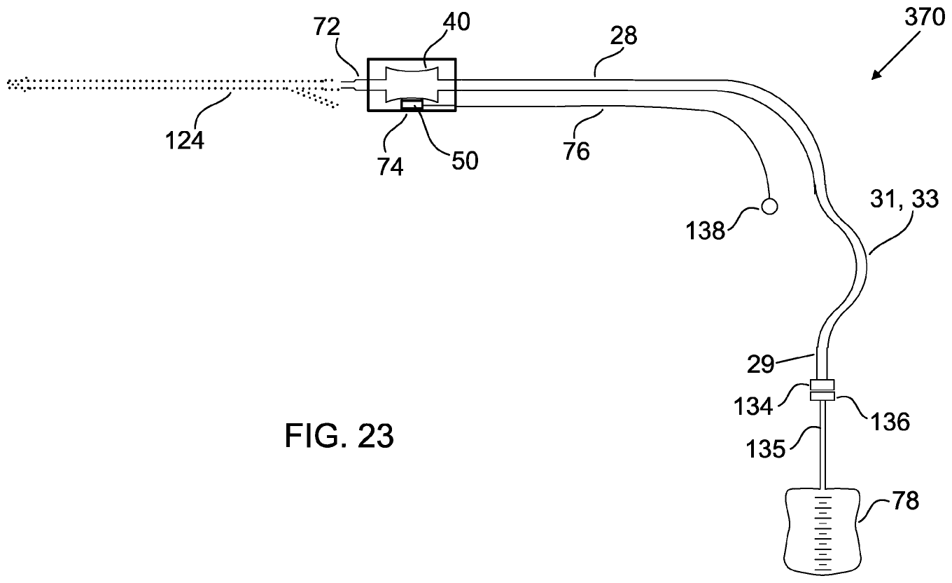

Reference is now made to FIG. 22, which is a schematic illustration of system 96 in accordance with some embodiments of the present invention. Reference is further made to FIG. 23, which is a schematic illustration of disposable kit 370 per FIG. 22, in accordance with some embodiments of the present invention. (In other words, FIG. 23 shows disposable components of FIG. 22.) As opposed to FIG. 20, in FIG. 22, reservoir 40 is upstream from tube 28 (as in FIGS. 12A-B, for example). FIG. 22 also differs from FIG. 20 in that the downstream end of exit tube 29 is coupled to a spill-proof connector 134, which is configured to couple to a bag connector 136 connected to bag 78 via a connecting tube 135. Bag connector 136 is described below in the section entitled “Bag connector for replaceable fluid bag.”

(Notwithstanding the above, it is noted that connectors may be used as in FIG. 22, or a drainage valve may be used as in FIG. 20, regardless of the position of the reservoir.)

As shown in FIG. 23, signal-carrying element 76 may terminate at an electrical and/or optical connector 138 for connecting to the controller. Connector 138 may comprise, for example, electrical interface 428a (FIG. 67).

Reference is now made to FIG. 67, which is a schematic illustration of reciprocating pump 20a together with conduit section 31, in accordance with some embodiments of the present invention.

FIG. 67 shows several techniques per which, when the urine-pumping device is coupled to the conduit section, a (non-disposable) sensor belonging to the urine-pumping device (e.g., by virtue of being coupled to case 348) may sense a parameter that varies with the amount of urine in the conduit section or an upstream portion of the conduit, such as a reservoir. (Typically, only one of these techniques is implemented in any given embodiment.) As noted above with reference to FIGS. 12A-B, although FIG. 67 shows reciprocating pump 20a, some of these techniques may be implemented with another type of pump, such as a peristaltic pump.

Per one such technique, pressure-conveying lumen 84 conveys a fluid pressure that varies with the amount of urine in an upstream reservoir, as described above with reference to FIGS. 13-15. Pressure sensor 88 is connected to pressure-conveying tube 406. As the urine-pumping device is coupled to the conduit section, tube 406 couples to connection port 86 (e.g., by sliding through the connection port), thereby effectively extending pressure-conveying lumen 84 such that the pressure sensor senses the fluid pressure in lumen 84.

Alternatively, the pressure sensor may sense a fluid pressure via a pressure-conveying tube coupled to the conduit section itself.

For example, pressure-measurement tube 410 may be connected to inlet port 338 of chamber 276 (or, in the case of a peristaltic pump or another positive-displacement pump, to a downstream portion of tube 28). Connection port 86 may be disposed at the end of pressure-measurement tube 410, and a diaphragm 430 may be disposed behind connection port 86, at any point along the pressure-measurement tube (e.g., at the end of the pressure-measurement tube, between the pressure-measurement tube and the inlet port). As the conduit section is coupled to the urine-pumping device, pressure-conveying tube 406 is coupled to connection port 86. Hence, changes in pressure in the conduit, which are due to changes in the volume of urine in pump chamber 276, in an upstream reservoir, and/or in the bladder, cause diaphragm 430 to distend toward or away from tube 406, thereby changing the fluid pressure in tube 406. These changes are sensed by pressure sensor 88.

Alternatively, connection port 86 may be coupled to the front wall of chamber housing 342 (i.e., the wall facing pump 20a), e.g., adjacent to diaphragm 344, and diaphragm 430 may be disposed behind the connection port. Following the sliding of tube 406 through connection port 86, changes in the volume of urine in the pump chamber cause diaphragm 430 to distend toward or away from tube 406, thereby changing the fluid pressure in tube 406. These changes are sensed by pressure sensor 88.

Alternatively, reservoir 40 may be disposed at inlet port 338 (or, in the case of a peristaltic pump or another positive-displacement pump, at the downstream portion of tube 28), and sensor 50 may monitor the reservoir, e.g., as described below in the section entitled “Example reservoirs and sensors.”

As yet another alternative, actuator 352 may function as a sensor.

For example, the actuator may measure the force exerted by the conduit on the pressing element (e.g., plunger 350), which varies as a function of the pressure within the conduit, and the controller may control the actuator responsively to the force. For example, for embodiments in which actuator 352 comprises a solenoid, the solenoid may sense changes in a magnetic field resulting from the varying force applied to plunger 350. Alternatively, a small amount of current, which is not enough to move the plunger, may be applied, and changes in the current may be measured.

As another example, the actuator may comprise an encoder configured to detect the position of the pressing element (e.g., plunger 350), which varies as a function of the pressure and/or volume within the conduit, and the controller may control the actuator responsively to the position.

Reference is again made to FIGS. 50 and 58.

In FIGS. 50 and 58, as in one embodiment shown in FIG. 67, pressure-measurement tube 410 is connected to the inlet of the pump, and diaphragm 430 is disposed between the pressure-measurement tube and the inlet (FIG. 50) or within the pressure-measurement tube (FIG. 58). A fluid-filled lumen, such as pressure-measurement lumen 288 (FIG. 50) or the lumen of pressure-conveying tube 406 (FIG. 58), is coupled to connection port 86, and a pressure sensor (e.g., within control unit 130) is coupled to the fluid-filled lumen so as to sense the pressure of the fluid, which varies with the internal pressure in the conduit.

As urine accumulates in the bladder, the increased pressure at the pump inlet deflects diaphragm 430 away from the inlet, such that the pressure in pressure-measurement lumen 288 or pressure-conveying tube 406 is increased. The controller detects this increase based on an output signal from the pressure sensor. In response to the pressure reaching a predetermined threshold, the controller may execute one or more pumping strokes.

Reference is now made specifically to FIG. 50.

Each stroke begins with diaphragm 430 at an initial position, and pressure-measurement tube 410 and pressure-measurement lumen 288 at an initial pressure.

In some embodiments, in each stroke, the controller first drives actuator 352 to decrease the pressure in cylinder 360, such that piston 358 is retracted (i.e., the pump chamber expands) and urine is suctioned from the bladder into the pump chamber through inlet valve 274. (In some cases, the suctioning of urine from the bladder empties the bladder.) Subsequently, the controller drives the actuator to increase the pressure, such that the piston is advanced. Due to the resulting increased pressure in the pump chamber, inlet valve 274 closes, outlet valve 284 opens, and urine flows out of outlet port 340, through urine lumen 292, and into urine-collection bag 78.

The pumping of the urine causes the pressure in the bladder to decrease. As a result of this decrease in pressure, diaphragm 430 distends from its initial position toward the inlet port, and hence, the volume in pressure-measurement tube 410 and pressure-measurement lumen 288 increases. As a result of this increase in volume, the pressure in pressure-measurement tube 410 and pressure-measurement lumen 288 decreases from its initial value.

Following the pumping, the bladder begins to refill with urine produced by the kidneys, such that the pressure at inlet port 338 is increased, diaphragm 430 returns to its initial position, and pressure-measurement tube 410 and pressure-measurement lumen 288 return to their initial pressure. In response to the returning to the initial pressure, the controller initiates another set of one or more strokes.

In other embodiments, in each stroke, the controller first advances the piston, thereby pumping urine out of the pump chamber as described above. Subsequently, the controller retracts the piston by reducing the pressure in cylinder 360, thereby suctioning more urine into the pump chamber.

In some embodiments, the number of strokes is predefined. In other embodiments, the controller executes a series of one or more strokes until the pressure in pressure-measurement tube 410 and pressure-measurement lumen 288 reaches another predefined threshold value.

In some embodiments, diaphragm 430 is elastic. In such embodiments, following each stroke, the diaphragm exerts a suction force on the bladder such that, advantageously, urine is drawn from the bladder even while the pump is idle. In other embodiments, diaphragm 430 is not elastic. However, even in such embodiments, the negative pressure in pressure-measurement tube 410 and pressure-measurement lumen 288 exerts a suction force on the bladder such that, advantageously, urine is drawn from the bladder even while the pump is idle.

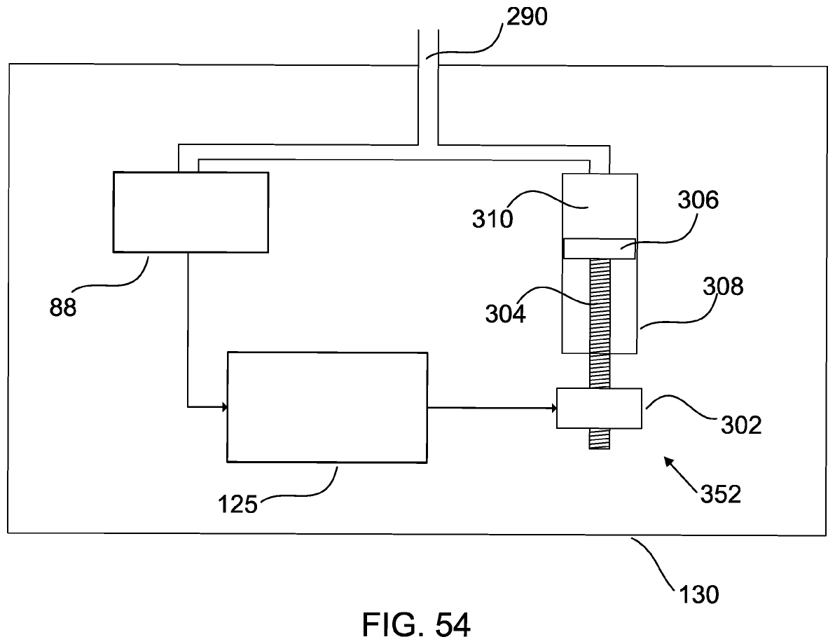

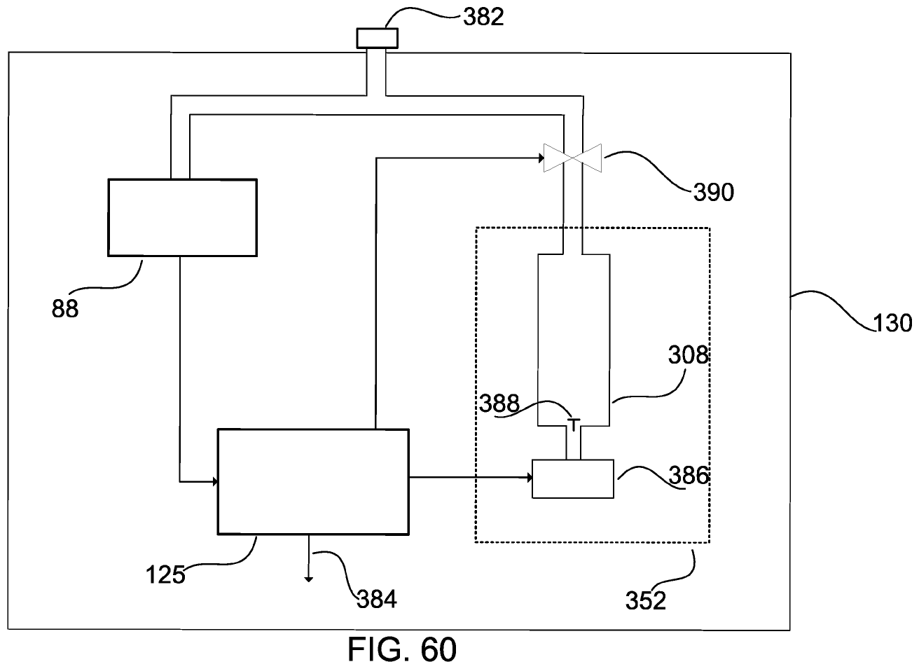

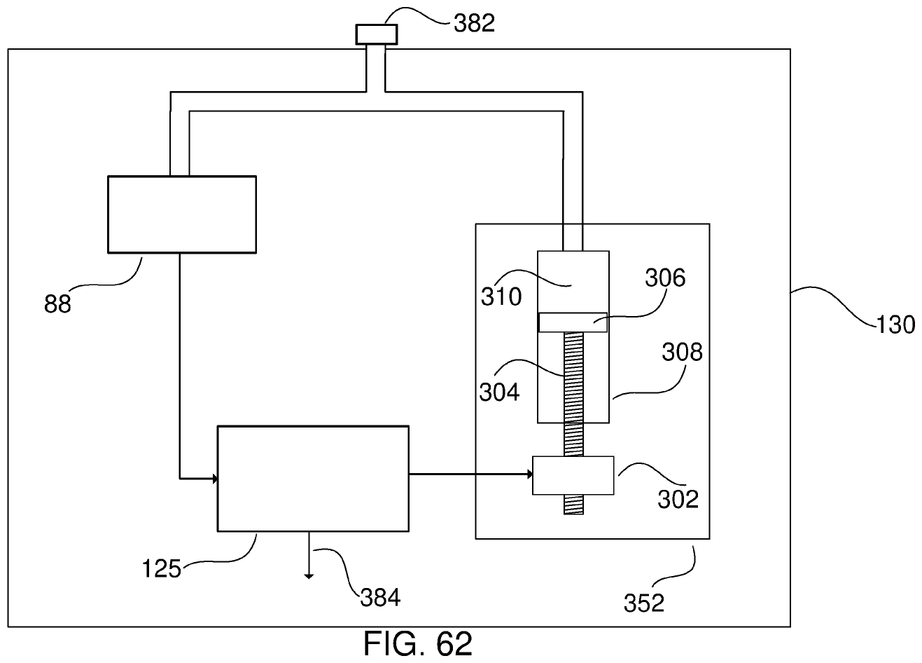

Reference is now made to FIG. 51, which is a schematic illustration of system 96, in accordance with some embodiments of the present invention. Reference is further made to FIG. 52B, which is a schematic illustration of conduit section 31, in accordance with some embodiments of the present invention. Reference is also made to FIG. 54, FIG. 60, and FIG. 62, which are schematic illustrations of control unit 130, in accordance with the embodiments of FIGS. 51 and 52B.

FIGS. 51 and 52B are similar to FIGS. 50 and 52A, respectively, in that moveable wall 343 (comprising piston 358 or diaphragm 344) is moved by pneumatic or hydraulic force. However, in FIGS. 51 and 52B, conduit section 31 does not comprise diaphragm 430 or pressure-measurement tube 410. Instead, pressure sensor 88 is coupled to the same fluid-filled lumen, such as pressure-application lumen 290 of tube 294, via which the pneumatic or hydraulic force is delivered. (Thus, tube 294 may be shaped to define only two lumens, rather than three.) The pressure sensor senses the pressure in the fluid-filled lumen (which varies with the internal pressure in the conduit), and communicates, to the controller, a signal indicating the pressure. In response thereto, the controller controls the actuator as described above with reference to FIG. 50.

In particular, as urine accumulates in the bladder, the increased pressure in inlet port 338 causes inlet valve 274 to open, such that the pressure in pump chamber 276 is also increased. This increased pressure pushes the moveable wall outward (i.e., causes the pump chamber to expand), thus increasing the pressure in cylinder 360, connection port 282, and lumen 290. This pressure is sensed by the pressure sensor, and in response to the increased pressure, the controller executes one or more pump strokes as described above with reference to FIG. 50. Following the final stroke, cylinder 360 is fixed at a predetermined pressure, by operating the actuator in response to feedback from the sensor until the predetermined pressure is reached. Subsequently, as urine flows into the pump chamber, the pressure in the cylinder, and therefore in lumen 290, increases from the predetermined pressure, such that the magnitude of the increase (as measured by the pressure sensor) is indicative of the amount of urine flowing in.

In some embodiments, the pressure in pump chamber 276 is below atmospheric pressure both at the start of (i.e., immediately before) the strokes and at the end of (i.e., immediately after) the strokes. Alternatively, the pressure may be above atmospheric pressure at the start of the strokes, but below atmospheric pressure at the end of the strokes. Alternatively, the pressure may be above atmospheric pressure both at the start of the strokes and at the end of the strokes.

(FIG. 54 is similar to FIG. 53, FIG. 60 is similar to FIG. 57, and FIG. 62 is similar to FIG. 59, except for pressure sensor 88 and actuator 352 being connected to a single fluid-filled lumen, e.g., via a single connector 382.)

Alternatively to the example embodiments shown in FIGS. 50, 51, and 52A-B, system 96 may comprise any other suitable pneumatically-actuated or hydraulically-actuated reciprocating pump that receives a driving force from actuator 352 through pressure-application lumen 290 and/or one or more other lumens.

Reference is now made to FIG. 61, which is a schematic illustration of system 96, in accordance with some embodiments of the present invention.

FIG. 61 is similar to FIG. 58, in that system 96 comprises a compound actuator. However, in FIG. 61, pressure-conveying tube 406 is omitted, and instead connection port 86 is coupled to fluid-filled tube 291 via a connecting tube 402. Fluid-filled tube 291 is coupled to both pressure sensor 88 and actuator 352, e.g., as shown in FIG. 54, FIG. 60, or FIG. 62. Thus, as the pressure within tube 291 varies with the internal pressure in the conduit, these variations in pressure are sensed by the pressure sensor.

In other embodiments, instead of sensing a fluid pressure via a fluid-filled lumen, pressure sensor 88, which is typically disposable, couples to the conduit or to the urinary catheter so as to sense the internal pressure in the conduit or the pressure at the outlet of the urinary catheter. To facilitate this, connection port 86 may be coupled to any portion of the conduit, such as any of the tubes belonging to the conduit or to conduit section 31, and may couple to the pressure sensor such that the pressure sensor senses the internal pressure in the portion of the conduit. In such embodiments, the pressure sensor is configured to communicate, to the controller, a signal indicating the pressure, and the controller is configured to control the pumping of urine responsively to the signal.

In this regard, reference is now made to FIG. 30, which is a schematic illustration of system 96 in accordance with some embodiments of the present invention.

In some embodiments, pressure sensor 88 senses the pressure at the outlet of urinary catheter 124 (e.g., via a T-connector connecting the outlet of the catheter to the catheter connector, as shown in FIG. 30), and communicates a signal indicating the pressure to controller 125. The controller controls the pump (which may comprise peristaltic pump 20 or any other type of positive-displacement pump) in response to the signal. For example, upon the pressure reaching a predetermined threshold due to accumulation of urine in the bladder, the controller may cause the pump to execute one or more strokes. (In effect, in such embodiments, the bladder itself functions as an upstream reservoir.)

In other embodiments, pressure sensor 88 couples to the conduit so as to sense a pressure in the conduit, and communicates a signal indicating this pressure to controller 125. For example, the pressure sensor may be coupled to tube 28, e.g., at the downstream thereof within control unit 130.

Alternatively to the example embodiments shown in FIGS. 50-51, 53-54, and 57-62, system 96 may comprise any other suitable pneumatic or hydraulic actuator that transmits a driving force to conduit section 31 or to actuator 396 through one or more lumens.

FIG. 67, to which reference is again made, also shows a technique by which signal-carrying element 76 (FIG. 12A) may be connected to the controller. In particular, in some embodiments, a first electrical interface 428a is coupled to the conduit, e.g., to conduit section 31, and is configured to connect (via signal-carrying element 76) to a sensor, such as an upstream sensor 50 monitoring a reservoir or a pressure sensor 88. The force-applying elements of the urine-pumping device, such as plunger 350 and actuator 352, are coupled (e.g., via case 348) to a second electrical interface 428b connected to the controller. (The connection to the controller is not shown in FIG. 67.) Second electrical interface 428b is configured to contact first electrical interface 428a, when conduit section 31 is coupled to the force-applying elements, such that the sensor communicates a signal to the controller via the first electrical interface and second electrical interface. In other words, as the conduit section is coupled to the urine-pumping device, an electrical connection between electrical interface 428a and electrical interface 428b is established, such that the sensor signal may be communicated to the controller.

Interface 428a and/or interface 428b may be flexible and/or springy. In some embodiments, one of the interfaces comprises a spring-loaded (“pogo pin”) connector, and the other interface comprises an electrical contact.

### Example Reservoirs and Sensors

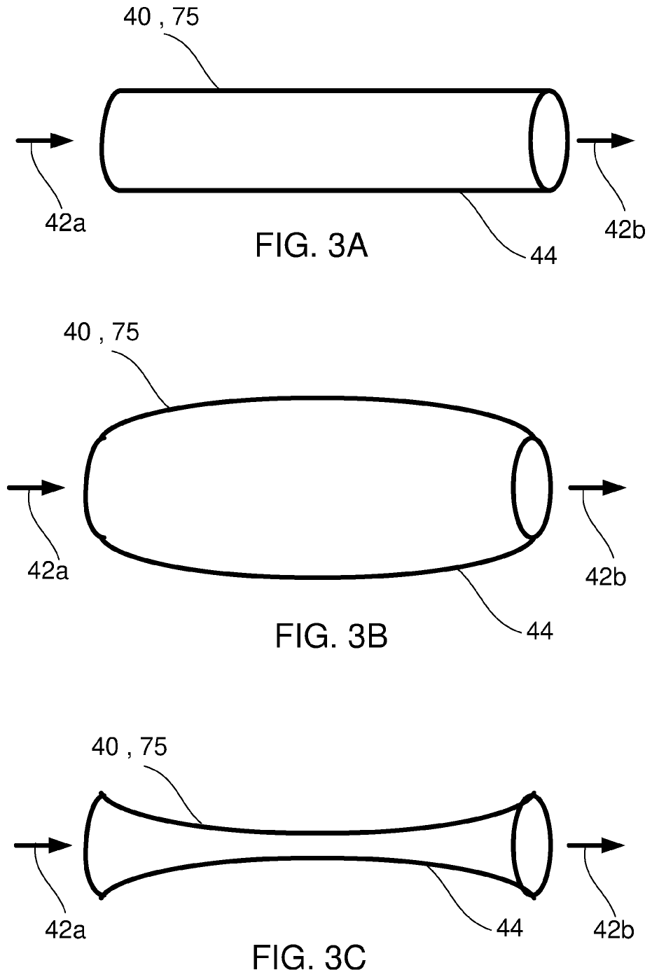

Reference is now made to FIGS. 3A-C, which are schematic illustrations of reservoir 40 in different respective states, in accordance with some embodiments of the present invention. First flow indicators 42a indicate the flow of urine into the reservoir, and second flow indicators 42b indicate the flow of urine from the reservoir.

As described above with reference to FIGS. 12A-B, in some embodiments, reservoir 40 comprises expandable tube 75. In some embodiments, the expandability of tube 75 is due to at least a portion of the tube having greater elasticity relative to other tubes (e.g., tube 28) belonging to the disposable kit. For example, at least a portion of the wall 44 of tube 75 may be thinner than that of the other tubes.

FIG. 3A shows tube 75 in its resting state, which the tube assumes when the internal pressure within the tube is equal to atmospheric pressure. FIG. 3B shows the tube in an expanded (or “inflated”) state, which the tube assumes when the internal pressure is greater than the atmospheric pressure, e.g., due to accumulated urine in the tube. FIG. 3C shows the tube in a deflated state, which the tube assumes when the internal pressure is less than the atmospheric pressure.

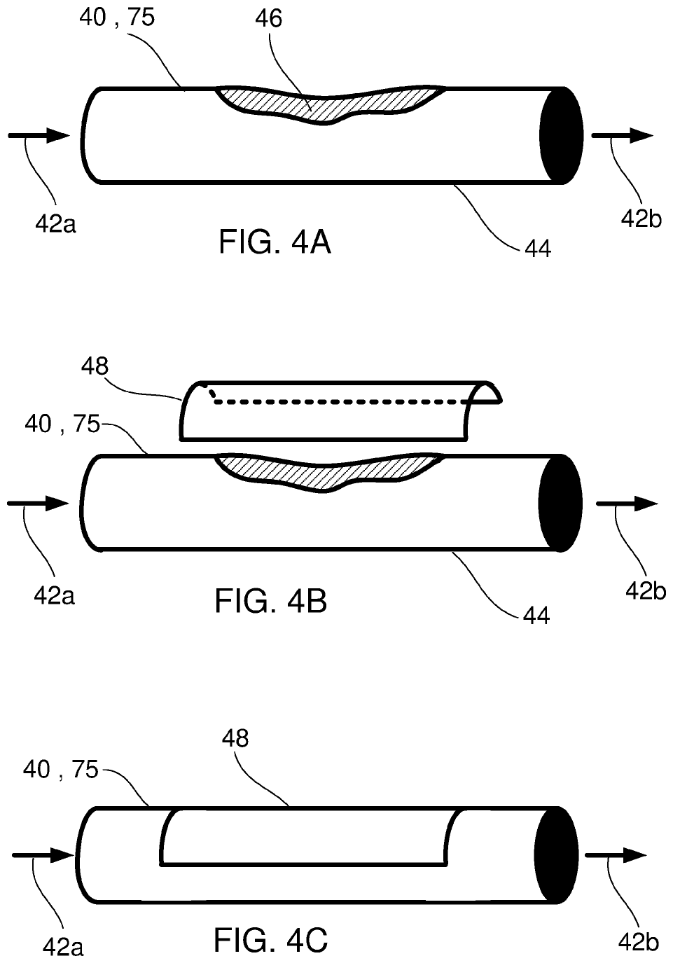

Reference is now made to FIGS. 4A-C, which are schematic illustrations of reservoir 40, in accordance with some embodiments of the present invention.

In some embodiments, wall 44 does not necessarily have greater elasticity. Rather, wall 44 is shaped to define an opening 46 (FIG. 4A), and tube 75 further comprises a flexible (elastic) diaphragm 48 (FIG. 4B). Diaphragm 48 is configured to couple to wall 44 over opening 46 (FIG. 4C), such that tube 75 is expandable by virtue of the flexibility of the diaphragm.

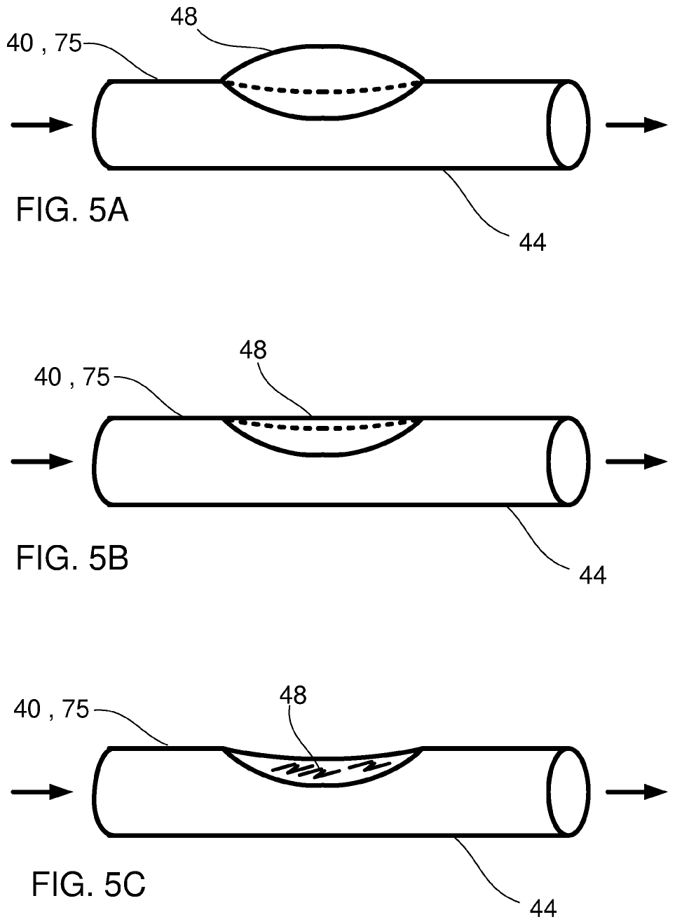

In this regard, reference is now further made to FIGS. 5A-C, which are schematic illustrations of reservoir 40, as shown in FIGS. 4A-C, in different respective states, in accordance with some embodiments of the present invention.

FIG. 5A shows the reservoir with diaphragm 48 expanded outward due to the internal pressure being greater than the atmospheric pressure. FIG. 5B shows the reservoir with the diaphragm in its relaxed state, which the diaphragm assumes when the internal pressure is equal to the atmospheric pressure. FIG. 5C shows the reservoir with the diaphragm collapsed inward due to the internal pressure being less than the atmospheric pressure.

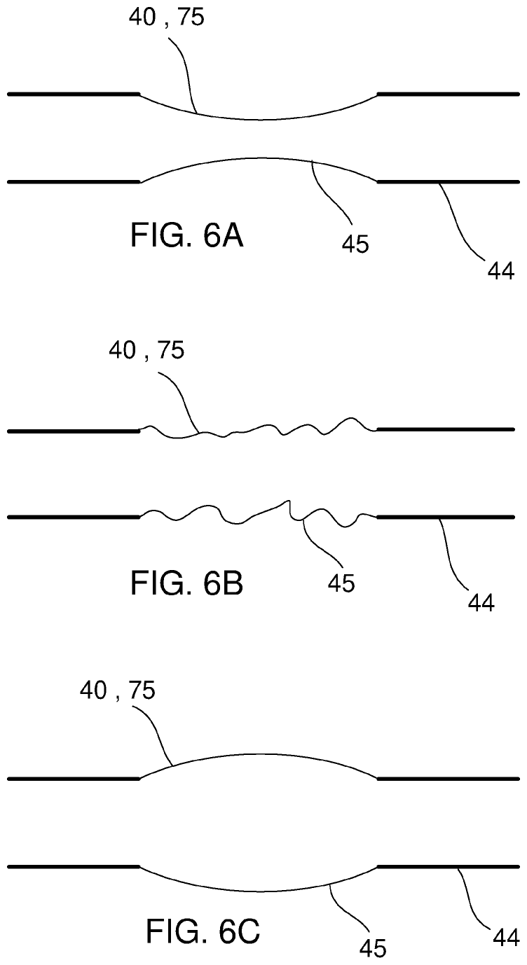

Reference is now made to FIGS. 6A-C, which are schematic illustrations of a longitudinal cross-section through reservoir 40 in different respective states, in accordance with some embodiments of the present invention.

In some embodiments, wall 44 comprises a material 45, such as nylon, that is not elastic, but rather, is creased, and tube 75 is expandable by virtue of material 45.

In particular, when reservoir 40 contains a smaller volume of urine, material 45 is collapsed inward, as shown in FIG. 6A. On the other hand, when the reservoir contains a larger volume of urine, material 45 is expanded outward, as shown in FIG. 6C. When reservoir 40 contains an intermediate volume of urine, material 45 is maximally creased and bulges neither inward nor outward, as shown in FIG. 6B.

(In the embodiments of FIGS. 3A-C and 4A-C, due to the elasticity of the tube wall or of diaphragm 48, reservoir 40 may apply positive and/or negative pressure to the bladder. On the other hand, in the embodiment of FIGS. 6A-C, the reservoir does not apply pressure to the bladder.)

For embodiments in which the conduit comprises an expandable portion, such as an expandable reservoir or pump chamber, the system may comprise sensor 50, which is configured to sense the degree of expansion of the expandable portion, i.e., the degree to which the expandable portion is expanded relative to the most compressed state of the expandable portion. In particular, the sensor senses a parameter that correlates with (and is thus indicative of) the degree of expansion, such as a position of a wall of the expandable portion.

In some embodiments, sensor 50 comprises an optical sensor configured to sense the degree of expansion of the expandable portion by emitting light at the expandable portion. In this regard, reference is now made to FIGS. 7A-C, 8A-C, 9, and 10A-C. By way of example, each of these figures assumes that the optical sensor is functionally coupled to reservoir 40 comprising expandable tube 75.

In some embodiments, as shown in FIGS. 7A-C, sensor 50 comprises a light source 52, configured to emit light 56 at reservoir 40 such that the light is reflected by the reservoir. (Optionally, as shown in FIGS. 8A-C, sensor 50 may further comprise at least one optical component 60, such as a lens, through which light 56 is emitted.) Sensor 50 further comprises a light detector 54, configured to detect the reflected light 58 and to generate a signal in response thereto.

Light source 52 and light detector 54 are disposed relative to one another such that the amount of reflected light 58 detected by the light detector varies as a function of the degree to which the reservoir is expanded or collapsed. In one such arrangement, light source 52 surrounds light detector 54.

Thus, when the reservoir is in its relaxed state as in FIG. 7A, a baseline amount of light is detected. When the reservoir is expanded as in FIG. 7B, more light is reflected away from the light detector due to the convexity of the reservoir; hence, the amount of light detected is less than the baseline amount. Conversely, when the reservoir is collapsed as in FIG. 7C, more light is reflected toward the light detector due to the concavity of the reservoir; hence, the amount of light detected is more than the baseline amount. Hence, the amount of detected light varies with the amount of urine in the reservoir.

Similarly to FIGS. 7A-C, FIGS. 8A-C show an embodiment in which sensor 50 emits light at the reservoir and generates a signal indicating the amount of the light that is detected. FIGS. 8A-C differ from FIGS. 7A-C, however, in that light source 52 and light detector 54 are disposed on opposite sides of the reservoir, such that the light detector detects light that is not reflected by the reservoir.

When the reservoir is in its relaxed state as in FIG. 8B, the light detector detects a baseline amount of light. When the reservoir is expanded as in FIG. 8C, the light detector detects less than the baseline amount. Conversely, when the reservoir is collapsed as in FIG. 8A, the light detector detects more than the baseline amount. Hence, the amount of detected light varies with the amount of urine in the reservoir.

In other embodiments, as shown in FIG. 9, sensor 50 comprises a fiber-optic core 62, which is oriented perpendicularly to reservoir 40 such that light 56 emitted through core 62 by light source 52 (FIGS. 8A-C) is reflected partially by the near wall 44a of the reservoir and partially by the far wall 44b of the reservoir. The phase difference dp between the two reflections, which is detected by light detector 54 (FIGS. 8A-C) indicates the degree to which the reservoir is expanded or collapsed, and hence, the amount of urine in the reservoir.

Similarly to FIGS. 7A-C, FIGS. 10A-C show an embodiment in which light source 52 and light detector 54 are disposed at the same side of reservoir 40 such that the amount of reflected light detected by the light detector varies with the amount of urine in the reservoir. However, in FIGS. 10A-C, the light source and light detector are spaced apart from one another and oriented obliquely with respect to the reservoir such that the overlap 64 between the area illuminated by light source 52 and the area from which light is reflected to light detector 54 varies with the degree to which the reservoir is expanded.

In particular, as shown in FIG. 10A, overlap 64 is an increasing function of the distance d0 between sensor 50 and the reservoir, which in turn depends on the degree to which the reservoir is expanded. Thus, for example, the overlap 64a when the reservoir is collapsed (FIG. 10B) is greater than the overlap 64b when the reservoir is in its resting state (FIG. 10C).

For embodiments in which sensor 50 comprises an optical sensor, the optical sensor may be further configured to sense a visual parameter (e.g., the color, opacity, and/or transparency) of the urine and to communicate, to the controller, a signal indicating the visual parameter.

Alternatively, a separate optical sensor may sense the visual parameter.

Reference is now made to FIGS. 11A-B, which are schematic illustrations of a contact sensor 50 functionally coupled to reservoir 40, which comprises expandable tube 75, in accordance with some embodiments of the present invention. (Alternatively, contact sensor 50 may be functionally coupled to any other expandable portion of the conduit.)

In some embodiments, sensor 50 comprises a conducting element 66, which is coupled to reservoir 40, and two electrical contacts 68. Conducting element 66 and electrical contacts 68 function as a binary switch, the state of which indicates whether the reservoir is expanded. In particular, when reservoir 40 is expanded as shown in FIG. 11A, the switch is closed, in that an electrical current may flow, via conducting element 66, between electrical contacts 68. On the other hand, when the reservoir 40 is not expanded as shown in FIG. 11B, the switch is open. (In view of above, this embodiment of sensor 50 is also referred to below as a “switch.”)

In other embodiments, sensor 50 comprises a pressure sensor configured to sense the pressure in a fluid-filled volume external to the reservoir, e.g., per any of FIGS. 13-15.

Alternatively, sensor 50 may be of any other suitable type, such as ultrasonic, capacitive, inductive, resistive, or electromagnetic.

Optionally, for any of the embodiments of sensor 50 described above, one or more components of the sensor (e.g., the entire sensor) may be disposable.

### Pump Control

The controller continually receives a signal that varies as a function of the amount of urine in the bladder of the subject or in the conduit connected to the urinary catheter that catheterizes the subject. As described above, the signal may be received from an optical sensor, a pressure sensor, or any other suitable type of sensor. The signal may indicate the pressure within the conduit (or within a fluid-filled tube coupled to the conduit), a degree of expansion of an expandable portion of the conduit, or any other parameter that varies with the amount of urine.

As further described above, in response to the signal (and, optionally, in response to one or more other inputs), the controller controls a pump, typically comprising a positive-displacement pump (e.g., a peristaltic pump or reciprocating pump), i.e., uses the pump to pump urine through the conduit. In some embodiments, the controller controls the pump so that the pressure within the conduit remains less than atmospheric pressure.

For example, using the pump, the controller may keep the volume of urine in the bladder relatively constant, e.g., within a range of 20 ml, e.g., within a range of 10 ml. One advantage of keeping the volume of urine relatively constant is that the subject's urine production (i.e., the amount of urine produced by the kidneys) may be more closely tracked in real-time.

As a specific example, the controller may pump the urine through the conduit such that the amount of urine in the bladder remains less than 20 ml, e.g., less than 10 ml. Keeping the bladder relatively empty facilitates measuring the intra-abdominal pressure of the subject, as further described below in the section entitled “Measuring intra-abdominal pressure (IAP).”

It is noted that the scope of the present invention includes using any pump—not necessarily a positive-displacement pump—to keep the volume of urine in the bladder relatively constant. Thus, for example, a pump that is not a positive-displacement pump may be used to keep the volume of urine in the bladder relatively constant, and the subject's urine output (which, due to the relatively constant volume in the bladder, may be approximately the same as the subject's urine production) may be measured manually, e.g., by noting the fill level of the urine-collection bag or another container, such as a graduated cylinder.

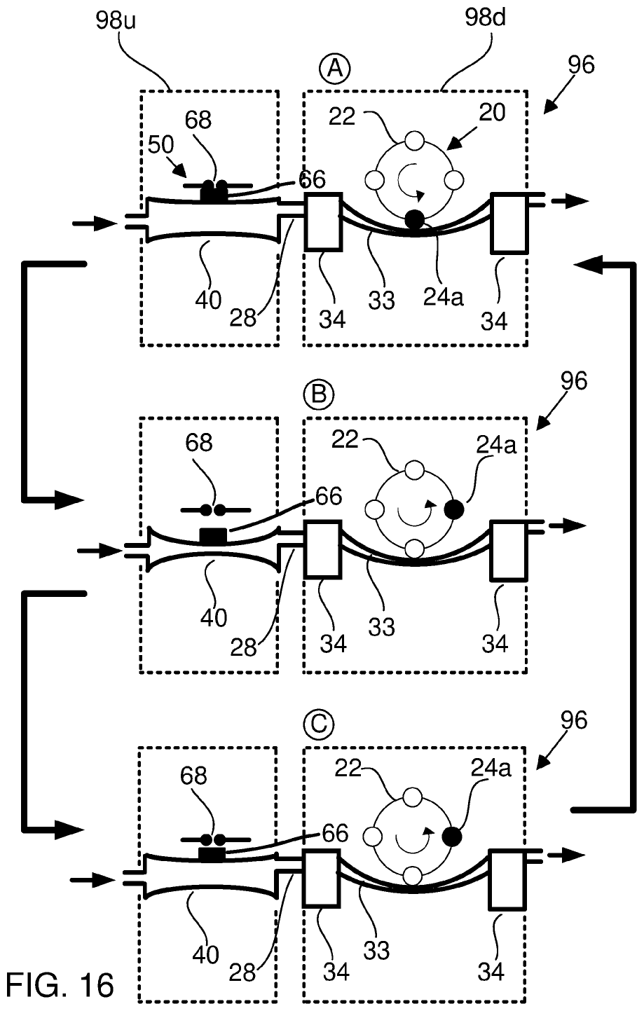

In this regard, reference is now made to FIG. 16, which is a schematic illustration of the operation of system 96, in accordance with some embodiments of the present invention. By way of example, FIG. 16 shows an embodiment in which the disposable kit comprises reservoir 40 and a binary switch 50, and the urine-pumping device comprises peristaltic pump 20. For simplicity and ease of illustration, some components of system 96 are omitted from FIG. 16, and system 96 is instead shown as comprising an upstream module 98u, which comprises reservoir 40 and sensor 50, and a downstream module 98d, which comprises pump 20 and peristaltic pump tube 33. Urine flows from upstream module 98u to downstream module 98d via tube 28 (FIG. 66A).

In stage A of the operation, reservoir 40 is filled with urine. The filling of the reservoir causes the signal from switch 50, which was previously low (0), to jump to high (1) and thus cross a predefined threshold (e.g., 0.5). In response to the signal crossing the predefined threshold, the controller activates the pump.

For example, in the case of a rotary peristaltic pump, the controller may execute a pumping stroke by turning rotor 22 (e.g., counterclockwise) such that a roller 24a pushes a known volume of urine from peristaltic pump tube 33 further downstream, toward the urine-collection bag. (In the example shown, pump 20 comprises four rollers, such that the controller executes a one-quarter turn of rotor 22.) As the urine is pushed from peristaltic pump tube 33, an equivalent volume of urine flows downstream from reservoir 40.

In stage B of the operation, the pumping stroke has finished. Reservoir 40 is therefore collapsed due to the urine having been pumped from the reservoir, and switch 50 is open.

In stage C, reservoir 40 has begun to fill again, due to the flow of urine into the reservoir. Eventually, switch 50 is again closed, and the operation returns to stage A.

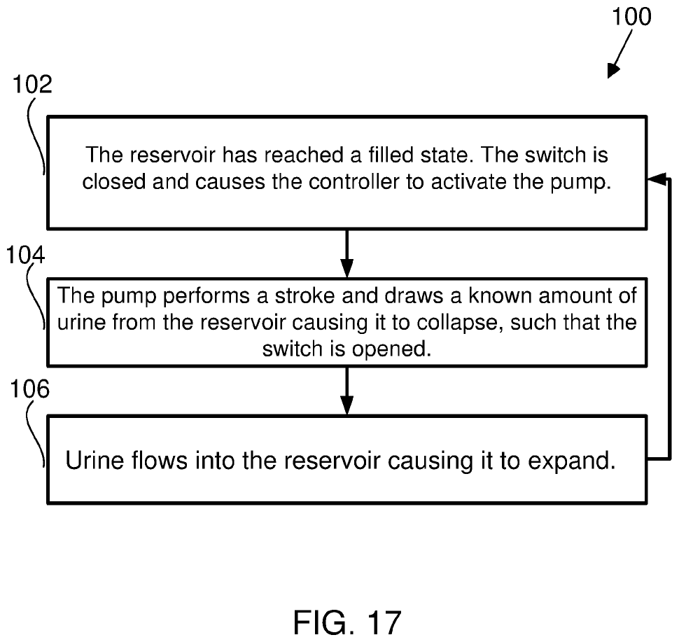

Reference is now made to FIG. 17, which shows a flow diagram 100 for the operation of system 96 per FIG. 16, in accordance with some embodiments of the present invention.

At a first step 102, the controller activates the pump, i.e., initiates a pumping stroke. At a second step 104, the pump performs a stroke, thereby drawing a known amount of urine from the reservoir. First step 102 and second step 104 correspond to stages A and B of FIG. 16.

At a third step 106, urine flows into the reservoir, as described above with reference to stage C of FIG. 16. The operation of the system then returns to first step 102.

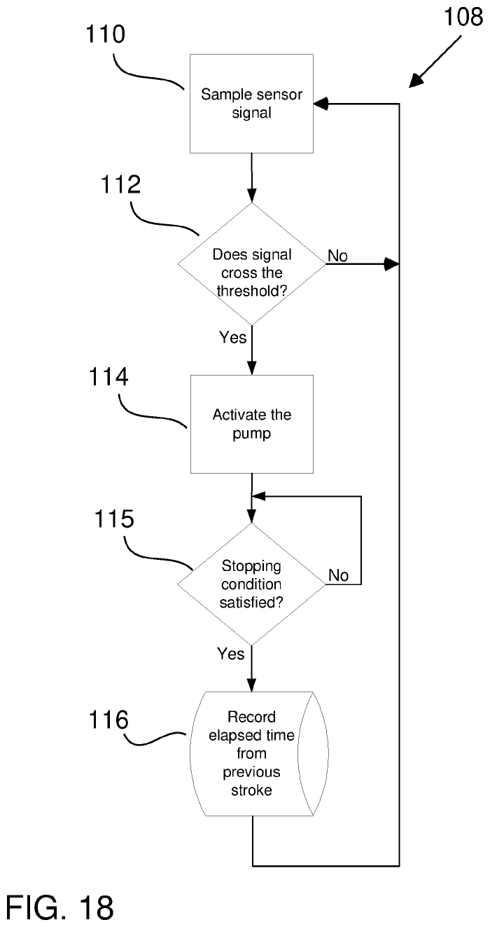

Reference is now made to FIG. 18, which shows a flow diagram for a control algorithm 108 executed by the controller, in accordance with some embodiments of the present invention. Algorithm 108 generalizes the control principles introduced above with reference to FIGS. 16-17.

At a signal-sampling step 110, the controller samples the signal received from the sensor. Subsequently, at an assessing step 112, the controller assesses whether the signal has crossed a predefined threshold. For example, in the case of a binary switch as in FIG. 16, the controller may assess whether the signal is high or low. In the case of an optical sensor monitoring an expandable reservoir, the controller may assess whether the amount of detected light crosses the threshold. In the case of a pressure sensor, the controller may assess whether the sensed pressure crosses the threshold.

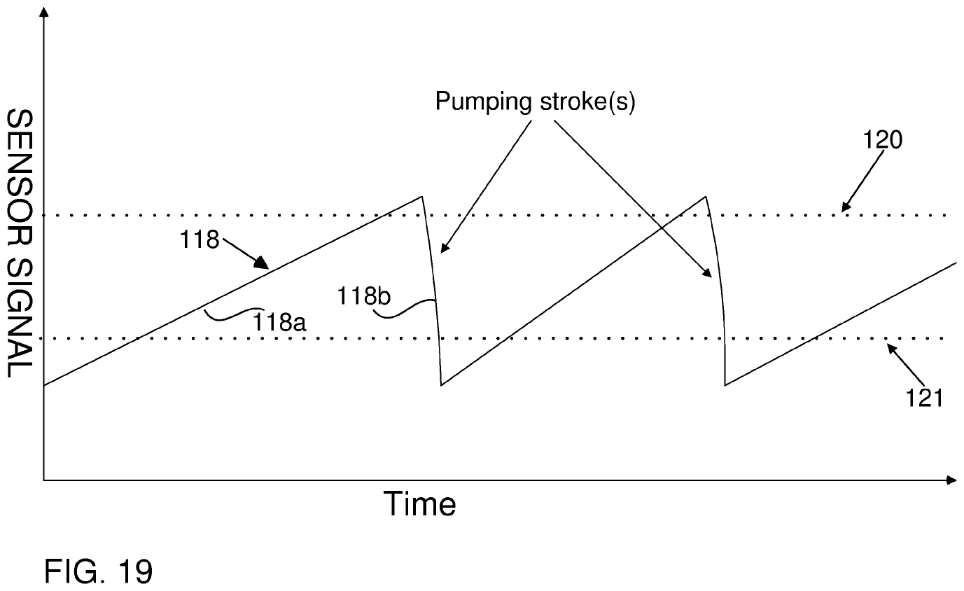

If not, the controller returns to signal-sampling step 110 (optionally following a wait period of predefined duration). Otherwise, the controller, at a pump-activating step 114, activates the pump, such that the pump begins pumping. Subsequently, at a checking step 115, the controller checks whether the predefined condition for stopping the pump is satisfied, e.g., as further described below with reference to FIG. 19. If not, the controller repeats checking step 115. Otherwise, the controller stops the pump and records the elapsed time from the previous stroke(s) at a recording step 116; this elapsed time may be used to calculate the pumped volume and/or the subject's rate of urine production. (The controller may also record other parameters used for these calculations, as described below in the section entitled “Calculating the pumped volume.”) The controller then returns to sampling step 110.

In other embodiments, the controller simply causes the pump to execute a predefined number of strokes (e.g., a single stroke as in FIG. 16). In such embodiments, at checking step 115, the controller may check whether the predefined number of strokes were executed.

For further details regarding the operation of system 96, reference is now made to FIG. 19, which is an example plot of a sensor signal 118 (indicating, for example, the volume of urine within a reservoir or the pressure at the outlet of the urinary catheter) as a function of time, in accordance with some embodiments of the present invention.

A first portion 118a of signal 118 corresponds to the gradual filling of the bladder or a portion of the conduit (e.g., a reservoir or pump chamber) with urine produced by the subject. Upon the signal crossing a first predetermined threshold 120, the controller activates the pump such that the pump begins pumping urine through the conduit, as indicated by a second portion 118b of the signal.

In some embodiments, as illustrated in FIG. 19, the controller causes the pump to pump a predefined number of strokes. Following the predefined number of strokes, urine again accumulates until the pump is triggered again.

In other embodiments, the controller stops the pump in response to the signal crossing first threshold 120 in the opposite direction. For example, if the pump was activated in response to the signal exceeding the first threshold, the pump may be stopped in response to the signal dropping below the first threshold. (In such embodiments, signal 118 remains within a narrow range straddling first threshold 120.)

Alternatively, the controller may stop the pump in response to the signal crossing a second predefined threshold 121 in the second direction after crossing the first threshold 120 in the second direction. For example, if the pump was activated in response to the signal exceeding the first threshold, the pump may be stopped in response to the signal dropping below second threshold 121 after dropping below first threshold 120.

Alternatively, the controller may stop the pump in response to the pump having pumped a predefined volume of urine.

In some embodiments, the controller redefines first threshold 120 and/or second threshold 121 dynamically, e.g., so as to balance the objective of keeping the bladder relatively empty (e.g., with an amount of urine less than 20 ml) with the objective of keeping the bladder tissue from clogging the urinary catheter. For example, if the controller ascertains that the bladder tissue was sucked into the catheter (e.g., as described below in the section entitled “Pressure and flow regulation and suction relief”), the controller may raise first threshold 120 and/or second threshold 121.

### Calculating the Pumped Volume

Typically, the pump is calibrated in advance and the results of the calibration are stored, e.g., in NVM 190 (FIG. 31). Using these calibration results, the controller may precisely calculate the pumped volume of urine.

Typically, the calibration results include a function or lookup table that maps one or more parameters to a pumped volume of urine. The parameters may include the “size” of the stroke, such as (i) the angle by which the rotor of a peristaltic pump was rotated, (ii) the distance by which a plunger was advanced against a moveable wall of a pump chamber, or (iii) the amount by which a pneumatic or hydraulic pressure pressing against the moveable wall was increased. The parameters may also include one or more of the ambient temperature, the pressure upstream from the pump, the pressure downstream from the pump, the elapsed time from the previous stroke, urine temperature, urine composition, and urine viscosity.

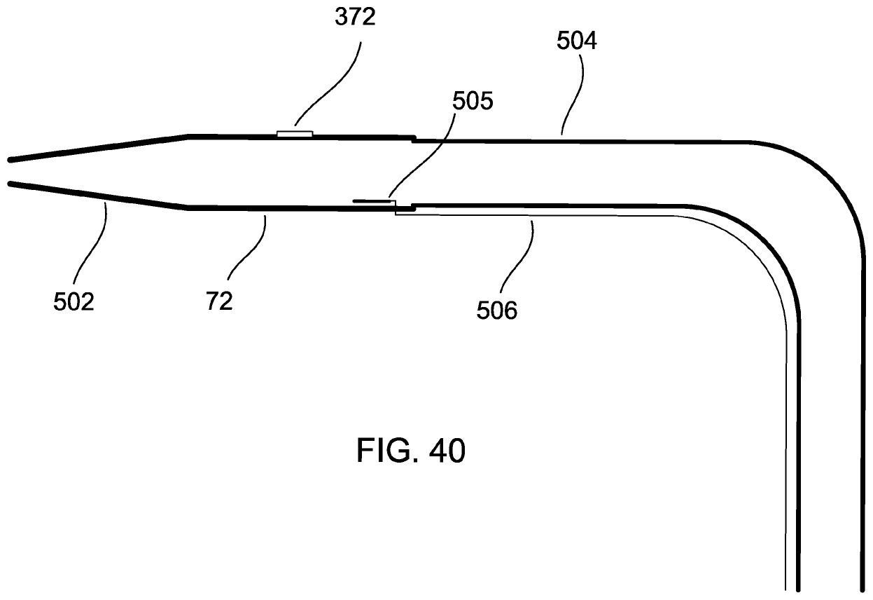

The ambient temperature may be determined by a temperature sensor in the control unit. The urine temperature may be determined by a temperature sensor placed inside conduit 371 (FIG. 66A), e.g., inside the catheter connector per FIG. 40, or fixed to the outside of the conduit.

To determine the pressure upstream and/or downstream from the pump, the pump inlet and/or outlet may comprise a thin wall, and a strain gauge may measure the deformation of the thin wall, which is a function of the pressure. Alternatively, the deformation may be measured in other ways, e.g., as described above for the various sensors included in the scope of the present invention. Alternatively, the downstream pressure may be calculated based on the amount of electric current consumed by the pump actuator during each pump stroke. (A higher current indicates greater resistance, and hence a higher downstream pressure.)

The elapsed time from the previous stroke may be tracked and recorded by the controller during operation.

The urine composition may be determined by spectroscopy and/or microscopy. The urine viscosity may be determined by processing images of the urine taken by a camera upstream from the pump during and following a pump stroke. (Such processing may be done by the controller or by a separate processor.) In particular, based on the images, a flow profile may be computed, and the viscosity may be calculated based on the speed and duration of the flow. (Less viscous fluids flow more quickly over shorter period, while more viscous fluids flow more slowly over longer periods.) Alternatively or additionally, the viscosity may be calculated based on the profile of pressure change during and immediately following the stroke. (Less viscous fluids undergo greater pressure changes more rapidly, while more viscous fluids undergo smaller pressure changes more slowly.)

For a peristaltic pump, the parameters may further include the physical dimensions and shore hardness of the peristaltic pump tube, the amount of time the tube remained squeezed in the pump, and the number of previous strokes the tube experienced. The physical dimensions and shore hardness of the tube may be determined at production and may be stored in the disposable kit, such as in or on cartridge 374 (FIG. 66A). The medium of storage may include a barcode, a quick response (QR) code, an engraved or printed string of characters, a non-volatile memory (e.g., a read-only memory (ROM)), or a radio frequency identification (RFID) tag. The control unit may comprise a reader configured to read this information from the storage medium. The amount of time the tube remained squeezed, and the number of previous strokes the tube experienced, may be tracked and recorded by the controller during operation.

Similarly, for a reciprocating pump configured to press against a diaphragm (e.g., per FIGS. 63A-B), the parameters may further include the physical dimensions and shore hardness of the diaphragm, the amount of time the diaphragm remained pressed, and the number of previous strokes the diaphragm experienced. These parameters may be determined and stored as described above.

Reference is again made to FIG. 66B.

In some embodiments, when a urine sample is required, a nurse (or any other user) submits an input to the controller (e.g., via display 378 and/or a keyboard) indicating the intended volume of the sample. In response thereto, the controller calculates the amount of time required for the designated urine volume to accumulate in the bladder, based on the current rate of urine production. After stopping the pump for this amount of time, the controller notifies the nurse (e.g., via display 378) that the sample may be taken via sampling port 372. After the nurse confirms that the sample was taken, normal pump operation is resumed. Subsequently, when computing the volume of urine production, the controller adds the sample volume to the total pumped volume.

In other embodiments, although the nurse may, optionally, enter an input indicating the nurse's intent to extract a urine sample, the nurse need not indicate the intended sample volume. Instead, as the urine is extracted, the controller causes the pump to pump urine upstream, thereby compensating for the extracted urine. For example, the controller may begin the upstream pumping in response to the upstream pressure dropping below a predetermined threshold (e.g., threshold 121 (FIG. 19)), and continue the upstream pumping so as to keep the pressure above the threshold.

### Pressure and Flow Regulation and Suction Relief

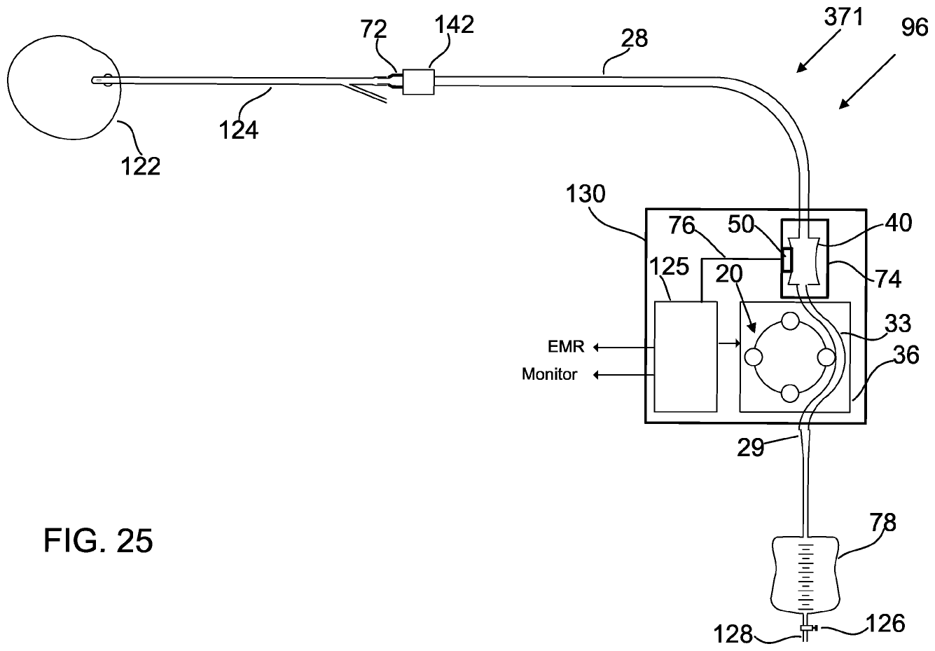

Reference is now made to FIG. 25, which is a schematic illustration of system 96, in accordance with some embodiments of the present invention.

In some cases, the wall of bladder 122 may be sucked into catheter 124, thereby inhibiting urine flow out of the bladder until enough urine (and hence, pressure) accumulates in the bladder so as to separate the bladder wall from the catheter. To address this challenge, some embodiments of the present invention provide a pressure valve 142, which is configured to reduce the force of suction on the bladder. Pressure valve 142 is typically coupled to the downstream end of catheter connector 72, e.g., between the catheter connector and tube 28. Alternatively, the pressure valve may be integral with the catheter connector.

It is noted that pressure valve 142 may be implemented regardless of whether urine is pumped or drained (via gravity) from the bladder. (When the urine is drained, the suction pressure on the bladder can be particularly high, e.g., 100 mbar; hence, the sucking of the bladder wall into the catheter may not only impede the flow of urine, but also cause harm to the bladder.)

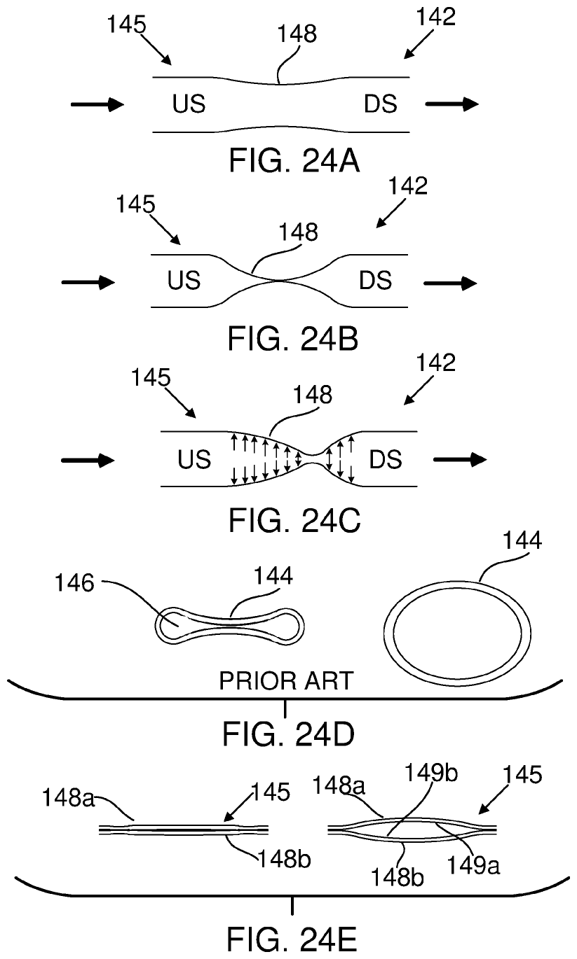

Reference is now additionally made to FIGS. 24A-C, which are schematic illustrations of pressure valve 142, in accordance with some embodiments of the present invention.

In some embodiments, conduit 371 comprises a first tube 145 configured to carry urine downstream from the bladder of the subject, and a second tube coupled to first tube 145 and configured to carry urine downstream from the first tube. For example, as shown in FIG. 25, the second tube may comprise tube 28, which is configured to carry urine downstream to a reservoir or pump. Alternatively, for embodiments in which tube 28 is omitted, the second tube may comprise exit tube 29 (or a multi-lumen tube having a urine lumen), which is coupled (via a pump) to the first tube and is configured to carry urine downstream to a urine-collection bag. Similarly, for embodiments in which the urine is drained, the second tube may be configured to carry urine downstream to a urine-collection bag.

First tube 145 functions as pressure valve 142, in that the first tube comprises one or more flexible walls 148 configured to collapse into the first tube, as the pressure within the first tube decreases, until the first tube is closed. The closing of the first tube isolates the upstream side (US) of the tube from the downstream side (DS), thereby relieving the upstream side from suction pressure (and also stopping the flow of urine through the tube).

For example, in FIG. 24A, the internal pressure within the pressure valve is relatively high (i.e., the suction pressure on the bladder is relatively low), such that the pressure valve is mostly open. In FIG. 24B, on the other hand, the internal pressure is lower (i.e., the suction pressure on the bladder is higher), such that the pressure valve is closed, thus isolating the upstream side of the valve from the downstream side.

In some embodiments, as shown in FIG. 24C, an upstream portion of flexible walls 148 is more flexible than is a downstream portion of flexible walls 148, e.g., due to the flexible walls being thinner upstream than downstream. Thus, even if the valve is closed, a relatively small increase in pressure on the upstream side due to the production of urine will cause the valve to reopen slightly and thus allow some urine to flow to the downstream side. Subsequently to the urine flowing to the downstream side, the pressure on the upstream side will decrease, and thus, the valve will close again. This process may be repeated any number of times until the pressure on the downstream side rises sufficiently to keep the valve open.

Reference is now made to FIG. 24D, which is a schematic illustration of a transverse cross-section through a prior-art tube 144 in inflated and deflated states. Reference is further made to FIG. 24E, which is a schematic illustration of a transverse cross-section through first tube 145 in accordance with some embodiments of the present invention.

The right side of FIG. 24D shows prior-art tube 144 in an open (relaxed) state at a relatively high internal pressure, while the left side of FIG. 24D shows prior-art tube 144 in a collapsed state at a lower internal pressure (higher suction). Even when tube 144 is collapsed, side channels 146 remain, such that tube 144 is unsuitable for use as a pressure valve.

First tube 145, on the other hand, is constructed differently from prior-art tube 144, such that the first tube is configured to collapse without leaving open side channels 146. For example, in some embodiments, flexible walls 148 comprise a first wall 148a, comprising a first face 149a, and a second wall 148b, comprising a second face 149b. Second face 149b is coupled to first face 149a at opposing edges of the first face such that, as the pressure between first wall 148a and second wall 148b decreases, the walls collapse toward one another until the first face and second face are fully in contact with one another between the edges, as shown at the left side of FIG. 24E. In this collapsed state, first tube 145 completely isolates upstream side US from downstream side DS.

In some embodiments, first tube 145 also functions as a reservoir, in that the first tube may expand as urine flows into the first tube. In such embodiments, sensor 50 may be placed adjacent to first tube 145, and the separate reservoir 40 shown in FIG. 25 may be omitted.

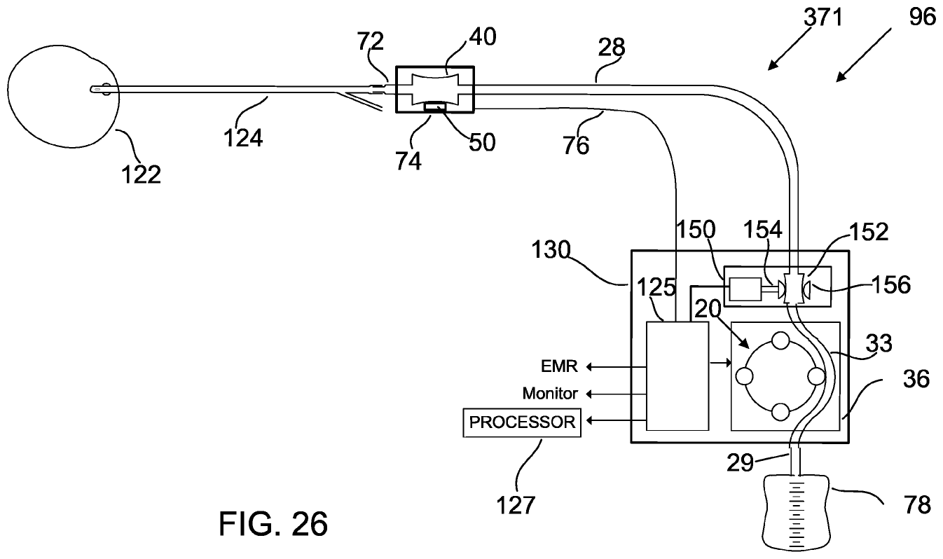

Reference is now made to FIG. 26, which is a schematic illustration of system 96 in accordance with some embodiments of the present invention.

In some embodiments, system 96 comprises a suction-relief mechanism 150 comprising a suction-relief tube 152, a plunger 154, and a counter-pressure fixture 156. Suction-relief tube 152 may be connected, for example, to the downstream end of tube 28, between tube 28 and the pump. Typically, suction-relief tube 152 is more flexible than is tube 28, e.g., by virtue of having thinner walls. It is noted that suction-relief mechanism 150 may be implemented regardless of whether urine is pumped or drained (via gravity) from the bladder.

In some cases, the controller may ascertain (e.g., based on the signal received from sensor 50) that the urine has at least partly ceased to flow downstream from bladder 122 through conduit 371. The cessation of flow may indicate that tissue of the bladder has been sucked into the urinary catheter.

In such embodiments, the controller, in response to identifying the cessation of flow, may stop pumping the urine. Furthermore, regardless of whether the urine is pumped or simply drained from the bladder, the controller may increase the pressure in the conduit so as to release the bladder tissue from the catheter.

For example, the controller may increase the pressure by pressing the conduit. For example, the controller may drive plunger 154 against suction-relief tube 152, such that the suction-relief tube is squeezed between the plunger and counter-pressure fixture 156. As the suction-relief tube is squeezed, urine may flow upstream, toward the bladder.

Subsequently to pressing the conduit, the controller may ascertain (e.g., based on the sensor signal) that urine has resumed flowing from the bladder. In response thereto, the controller may stop increasing the pressure, e.g., by stopping to advance the plunger. The controller may then gradually withdraw the plunger, thus allowing the suction-relief tube to re-expand; for example, the controller may withdraw the plunger at a rate proportional to the flow rate of urine into reservoir 40. For embodiments in which the urine is pumped, the controller, in response to ascertaining that urine has resumed flowing from the bladder, may resume pumping the urine downstream, e.g., following the withdrawal of the plunger.

For embodiments in which the urine is pumped, the controller may increase the pressure in the conduit upstream from the pump by operating the pump in reverse, i.e., in the upstream pumping direction, alternatively or additionally to using suction-relief mechanism 150. By operating the pump in reverse, the controller may cause urine to flow upstream.

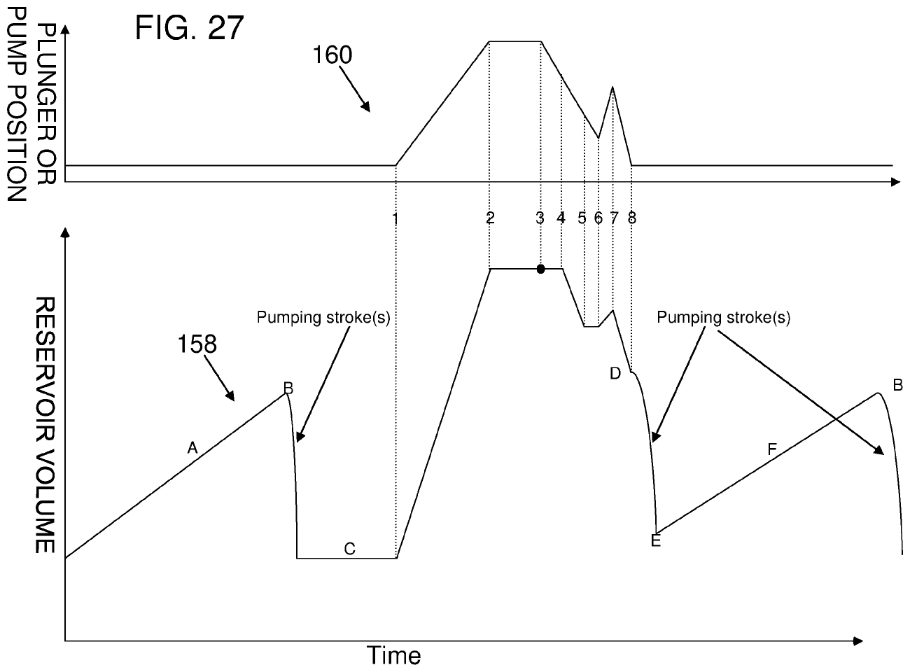

Reference is now further made to FIG. 27, which schematically illustrates an example performance of suction relief, in accordance with some embodiments of the present invention.

FIG. 27 shows a lower plot 158, which tracks the reservoir volume over time, and an upper plot 160, which tracks the position of plunger 154 (for embodiments in which the plunger is used to increase the pressure in the conduit) or of the pump (for embodiments in which the pressure is increased by reversing the pump).

As shown in plot 158, during period A, the reservoir is filled until the volume of the reservoir reaches a threshold value B. In response to the volume reaching the threshold, the controller causes the pump to pump one or more (forward) strokes, until the reservoir returns to its initial volume. Subsequently, as a result of the suction created by the pumping strokes, the bladder tissue blocks the urinary catheter such that, during period C, urine does not flow into the reservoir. In response to detecting the cessation of flow, the controller, from timepoint 1 to timepoint 2, drives the plunger against the suction-relief tube or pumps in reverse, thus causing the reservoir to expand beyond the threshold volume B. During this time, the controller registers the reservoir volume as a function of the position of the plunger or the position of the pump. In response to the reservoir volume increasing by more than the amount of urine pushed upstream by the plunger or pump, the controller may ascertain that the suction relief has succeeded.

In response to ascertaining that the suction relief has succeeded and to ascertaining, shortly after timepoint 2, that the reservoir is no longer expanding, the controller holds the plunger or pump in place until timepoint 3. Subsequently, the controller begins withdrawing the plunger or pumping in the forward direction. Initially, prior to timepoint 4, the reservoir volume remains constant, as any urine drawn from the reservoir is replaced with an equivalent volume from the bladder. Subsequently to timepoint 4, the bladder is empty, and the reservoir volume therefore decreases.

At timepoint 5, the bladder tissue is again sucked into the urinary catheter. As a result, the reservoir volume stops decreasing. In response thereto, at timepoint 6, the controller again begins to advance the plunger or to reverse the pump. At timepoint 7, the controller begins to withdraw the plunger or pump in the forward direction, until the plunger or pump reaches its initial position at timepoint 8. Subsequently, the controller operates the pump as usual.

As shown, at timepoint 8, the reservoir volume D is greater than the threshold B; therefore, the pump stroke(s) empty the reservoir to a volume E that is greater than the volume during period C. However, following period F, the volume reaches the threshold B, and the pump then empties the reservoir until the volume decreased to that of period C.

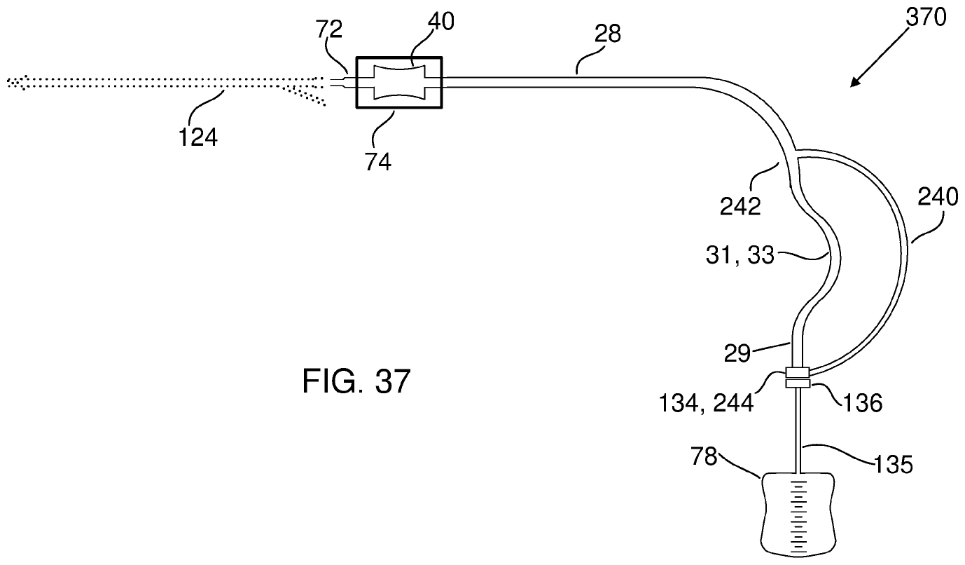

Reference is now made to FIG. 37, which is a schematic illustration of disposable kit 370 comprising a pressure-regulating bypass tube 240, in accordance with some embodiments of the present invention.

In some embodiments, disposable kit 370 comprises bypass tube 240. The upstream end of bypass tube 240 is connected to tube 28 (upstream from conduit section 31) at a junction 242. The downstream end of the bypass tube may be connected to a portion of the kit downstream from the conduit section, such as connector 134, exit tube 29, or urine-collection bag 78. Alternatively, the downstream end of the bypass tube may be connected to a separate urine-collection container.

In such embodiments, disposable kit 370 further comprises a pressure-relief valve 244 configured to prevent the flow of urine through the bypass tube when the pressure within the bypass tube is less than a predetermined threshold, and to allow the flow when the pressure exceeds the threshold, such that the urine bypasses the conduit section.

Hence, as long as the pressure is sufficiently low, valve 244 remains closed, such that the urine may be pumped through the conduit. On the other hand, in the event that the pump stops pumping urine (e.g., due to a mechanical fault in the pump, a fault in the controller, or a blockage in the conduit downstream from junction 242), the accumulation of urine causes a rise in pressure at the pressure-relief valve inlet. Upon the pressure passing the threshold, the pressure-relief valve opens, thereby allowing the downstream flow of urine through bypass tube 240.

For embodiments in which the bypass tube is connected to connector 134, such that the bypass tube passes between tube 28 and the connector, valve 244 may be integrated into the connector. Alternatively, regardless of whether the bypass tube is connected to connector 134, valve 244 may be coupled to any portion of the bypass tube, e.g., to the upstream end of the bypass tube at junction 242.

### Measuring Intra-Abdominal Pressure (IAP)

Reference is now made to FIG. 28, which is a schematic illustration of system 96, in accordance with some embodiments of the present invention.

By way of introduction, it is noted that the IAP of a subject may be measured by measuring the intra-bladder pressure of the subject at end-expiration when the bladder contains a predefined volume of fluid. Typically, this volume depends on the weight of the subject; for example, for an adult subject of average weight, the volume may be around 20-25 ml.

In some embodiments, controller 125 is configured to measure the subject's IAP in response to an instruction from a user. The instruction may be received via any suitable user interface to which the controller is connected, such as a touch screen belonging to display 378 (FIG. 66B) or a keyboard. The user may instruct the controller to measure the IAP once, or periodically at a rate defined by the user.

In such embodiments, the controller first empties bladder 122 by pumping urine from the bladder. (The bladder may be emptied even before the aforementioned instruction is received.) For example, the controller may empty the bladder based on a signal from pressure sensor 88 or sensor 50, as described above in the section entitled “Pump control.” The controller further calculates, based on the subject's urine-production rate, an estimated amount of time from the emptying of the bladder required for the predefined volume of urine to flow into the bladder from the subject's kidneys.

(Since, in practice, it may be impossible to literally empty the bladder of all urine, it should be understood that in the context of the present application, including the claims, using a pump to “empty” the bladder refers to pumping as much urine as possible from the bladder, e.g., such that the remaining volume of urine, which cannot be pumped from the bladder, is less than 20 ml, such as less than 10 ml.)

Subsequently to emptying the bladder, the controller refrains from pumping urine for the estimated amount of time (also referred to below as the “wait time”), thereby allowing urine to accumulate in the bladder and—for embodiments in which conduit 371 comprises reservoir 40—reservoir 40.

After the estimated amount of time has passed, the controller receives a signal that varies as a function of the pressure within the bladder. Based on the signal, the controller ascertains the pressure within the bladder.

For example, pressure sensor 88 may be coupled to the urinary catheter as described above with reference to FIG. 30, such that the pressure sensor senses the pressure at the outlet of the catheter. Provided the pressure sensor is at a predefined height such that the difference between the outlet pressure and the intra-bladder pressure is known, the controller may ascertain the pressure within the bladder based on the value of the signal.

Alternatively, pressure sensor 88 may be coupled to a fluid-filled tube in which the pressure varies as a function of the pressure within the bladder, e.g., per any of FIGS. 13, 50-51, 58, 61, and 67. In such embodiments, the controller may ascertain the pressure within the bladder by applying a function to the value of the sensor signal. The function may be calibrated in advance and stored in memory, e.g., NVM 190 (FIG. 31).

As yet another alternative, the controller may receive a signal from a sensor monitoring a reservoir, e.g., per any of FIGS. 12A-B, 20, and 22. Given that the reservoir expands as a function of the pressure in the bladder, the controller may ascertain the pressure within the bladder by applying a function to the value of the sensor signal. The function may be calibrated in advance and stored in memory, e.g., NVM 190 (FIG. 31).

Subsequently to receiving the signal, the controller may generate an output indicating that the pressure within the bladder, as indicated by the signal, is the IAP of the subject. The controller may then display the output on display 378 (FIG. 66B) or any other suitable display. Alternatively or additionally, the controller may communicate the IAP to another device or system such as a patient monitor, an EMR, a gateway, or a doctor's desktop computer, cellphone, or tablet computer.

For example, the output may include the numerical pressure value together with “IAP” or any other suitable explanatory string of characters. If the IAP is measured periodically, the output may include a plot of IAP over time.

Typically, the subject's breathing causes fluctuations in the intra-bladder pressure. As noted above, the IAP is measured at end-expiration, when the pressure reaches a local minimum. Hence, the controller may sample the signal periodically, e.g., at a rate of 10 times per second. (The controller may begin sampling the signal even before the wait time has transpired.) Based on the sampled pressure values (e.g., based on a frequency spectrum of the samples), the controller may estimate the breathing rate of the subject. Based on the estimated breathing rate, the controller may estimate a time to, following the wait time, at which an expiration will end. The controller may then select the first local minimum in pressure, within a predefined duration of t0, as the IAP.

Advantageously, this technique for IAP measurement does not necessitate injecting saline into the bladder, as required by conventional techniques. (It is noted that aside from the hassle and discomfort associated with the saline injection itself, the injection necessitates waiting 30-60 seconds for the subject's detrusor muscles to relax before the IAP is measured.)

Typically, for embodiments in which the controller keeps the bladder relatively empty during normal operation of the pump, the controller calculates the wait time based on the amount of urine pumped during a preceding period of time. For example, if a volume W of urine was pumped during a preceding period of length T, the controller may calculate the subject's rate of urine production as W/T. Denoting the target volume for IAP measurement as V, the controller may calculate the wait time as V*T/W.

In some embodiments, prior to generating the output, the controller verifies that the measured pressure is, in fact, the intraabdominal pressure. The controller then generates the output in response to the verification.

To perform the verification, the controller first re-empties the bladder, using the pump. The controller then ascertains that the amount of urine pumped from the bladder during the re-emptying deviates from the predefined volume V by less than a predefined threshold. In some embodiments, the predefined threshold is a percentage of V.

In some embodiments, the controller estimates the breathing rate of the subject based on the intra-bladder pressure signal (as described above), even without measuring the IAP. Alternatively or additionally, given that the intra-bladder pressure fluctuates with the subject's cardiac cycle, the controller may estimate the heart rate of the subject based on the intra-bladder pressure samples (e.g., based on a frequency spectrum of the samples). The breathing rate and/or heart rate may be displayed and/or communicated as described above for the IAP.

For further details regarding the IAP measurement, reference is now made to FIG. 29, which shows a flow diagram for an algorithm 164 for measuring IAP, in accordance with some embodiments of the present invention.

Algorithm 164 begins with a rate-determining step 166, at which the controller calculates the rate X of urine production, e.g., in units of ml/h, over a preceding period of time. Following the emptying of the bladder, the controller stops the pump at a pump-stopping step 168.

As described above, for IAP measurement, the volume of urine in the bladder must be allowed to reach a predetermined volume V, which, as indicated above, may be between 20 and 25 ml for some subjects. The controller therefore waits—i.e., keeps the pump stopped—for a period of time V/X, during a waiting step 170. For example, if X is in units of ml/h and V is in units of ml, the controller may wait 60*V/X minutes. Subsequently, at a pressure-measuring step 172, the pressure at the end of the subject's expiration is taken as the IAP.

Subsequently, at a resuming step 174, the operation of the pump is resumed. During another waiting step 176, the controller waits for the pump to stop, i.e., the controller waits until all the urine has been pumped from the bladder. Subsequently, at a calculating step 178, the controller calculates the amount of urine that was pumped from the bladder during waiting step 176. The controller then checks, at a checking step 180, whether the calculated amount is between V−α and V+α, where a is a predetermined percentage of V (e.g., 25%), for example. If the calculated amount is within these boundaries, the controller displays the IAP at a displaying step 182. Otherwise, the controller returns to stopping step 168, and repeats the measurement.

In other embodiments, the controller stops the pump for slightly more than the calculated wait time, e.g., such that an additional 5-15 ml accumulates in the bladder. Subsequently, the controller pumps urine from the bladder while sampling the intra-bladder pressure. Following the emptying of the bladder, the controller identifies the period in time at which the volume of urine in the bladder was V, based on the known volumes of urine pumped during each stroke. The controller then identifies, as the IAP, an intra-bladder pressure during this period at which the subject was at end-expiration.

In yet other embodiments, rather than waiting for a calculated wait time to transpire, the controller simply stops the pump and samples the intra-bladder pressure until the pressure stops rising. In response to ascertaining that the pressure stopped rising, the controller identifies the pressure at the next end-expiration event as the IAP.

In yet other embodiments, following the emptying of the bladder, the controller causes the pump to pump the volume V into the bladder. Following the 30-60 seconds required for the subject's detrusor muscles to relax, the controller identifies the pressure at the next end-expiration event as the IAP.

### Alerts

As described throughout the present application, in some embodiments, the controller is configured to pump urine from a bladder of a subject by controlling a pump. In such embodiments, the controller may be configured to generate an alert indicating a current or upcoming disruption to the pumping, which may include an inhibited flow of urine upstream or downstream from the pump. The alert may include a visual alert (e.g., a message displayed on a computer monitor and/or delivered to a cellphone) and/or an audio alert (e.g., beeping).

For example, the controller may generate an alert in response to an increased amount of power consumed by the pump, which indicates an increased resistance to the flow of urine downstream from the pump, e.g., due to collection bag 78 (FIG. 30) being full or due to a blockage in exit tube 29 (FIG. 30). (In the context of the present application, including the claims, a blockage in any particular conduit may include, for example, a kink in the tube or a solid body, such as a blood clot or kidney stone, lodged in the conduit.)

In other words, while the pump is in operation, the controller may monitor the amount of power consumed by the pump (in particular, by the actuator of the pump), e.g., by integrating the consumed current over time. The controller may further compare this amount of power to a baseline amount of power. (The baseline may change over time, e.g., due to peristaltic tube wear.) If the usage exceeds the baseline, the controller may generate an alert.

As another example, the controller may generate an alert in response to the collection bag being almost full, i.e., in response to the difference between the maximum capacity C of the collection bag and the pumped amount A of urine (as calculated by the controller) being less than a predefined threshold T. (It is noted that the controller need not necessarily explicitly calculate C−A and compare this difference to T; rather, the controller may simply compare A to C−T, and generate an alert in response to A exceeding C−T.)

In some cases, a blockage in the conduit upstream from the pump or in the urinary catheter may inhibit the flow of urine to the pump. Such a blockage may include, for example, a kink or a solid body such as a blood clot or a kidney stone. As described below, the controller may identify the existence of such a blockage using various techniques, and generate an alert accordingly.

For example, for embodiments in which the pump comprises a pump chamber (e.g., as in FIG. 67), the controller may monitor the volume of urine flowing into the pump chamber (e.g., based on the position of plunger 350). The controller may further calculate the minimum volume that is expected to flow into the pump chamber, given the operation of the pump and the expected production of urine. If the inflow is less than this estimate, the controller may generate an alert.

Alternatively, if the conduit includes a reservoir (e.g., as in FIG. 23), the controller may generate a blockage alert based on the amount of urine flowing into or out of the reservoir.

In particular, if the blockage is downstream from the reservoir, the controller may generate an alert in response to a signal indicating that the amount of urine that flowed from the reservoir (i.e., the gross outflow, which may be greater than the net change in the volume of urine in the reservoir) is less than the pumping volume of the pump, i.e., the volume that the pump would have pumped if the flow through the conduit were uninhibited. (The pumping volume may be calculated as described above in the section entitled “Calculating the pumped volume.”) For example, for embodiments in which the reservoir is expandable and sensor 50 (FIG. 23) detects the relative expansion or contraction of the reservoir, the controller may calculate the minimum amount by which the reservoir is expected to contract, given the operation of the pump and the expected production of urine. If the signal from sensor 50 indicates that the reservoir contracted by less than this estimate, the controller may generate an alert.

On the other hand, if the blockage is upstream from the reservoir, the controller may generate an alert in response to a signal indicating that an increase in the amount of urine in the reservoir is less than a predefined threshold. For example, the controller may calculate the minimum amount by which the volume of urine in the reservoir is expected to increase over a period of time, based on a recent rate of urine production. If, over the period of time (during which the pump is typically idle), the increase is less than this estimate, the controller may generate an alert.

Alternatively, if a pressure sensor is coupled to the conduit so as to sense the pressure in the conduit or a fluid pressure that varies with the pressure in the conduit (e.g., as in FIG. 58), the controller may generate a blockage alert based on the pressure.

In particular, if the blockage is downstream from the pressure sensor, the controller may generate an alert in response to a change in the pressure. For example, the controller may calculate the minimum amount by which the pressure is expected to decrease, given the operation of the pump and the expected production of urine. If the decrease in pressure is less than this estimate, the controller may generate an alert.

On the other hand, if the blockage is upstream from the pressure sensor, the controller may generate an alert in response to an increase in the pressure being less than a predefined threshold. For example, the controller may calculate the minimum amount by which the pressure is expected to increase over a period of time, based on a recent rate of urine production. If, over the period of time (during which the pump is typically idle), the increase is less than this estimate, the controller may generate an alert.

In this regard, reference is now made to FIG. 32, which shows a flow diagram for a controlling-and-alerting algorithm 200 executed by the controller, in accordance with some embodiments of the present invention. Controlling-and-alerting algorithm 200 includes sampling step 110, assessing step 112, and pump-activating step 114, as described above for algorithm 108 with reference to FIG. 18. In addition, per algorithm 200, the controller executes several series of steps in parallel to each other following pump-activating step 114. In particular:
(i) In a first series of steps, the controller checks for obstructions upstream from the pump, and alerts the user regarding any such obstructions.

First, the controller again samples the sensor at sampling step 110. The controller then checks, at a checking step 202, whether the sensor output indicates that the reservoir responded to the pump stroke, i.e., whether the outflow from the reservoir was within a predefined (small) deviation of the pumping volume of the pump. To estimate the outflow, the controller may estimate the volume of urine produced subsequent to the most recent pump activation, and subtract the net change in reservoir volume (which may be a negative number) from this estimated volume.

If the reservoir did not respond to the pump stroke, there is likely a kink (or another obstruction) in tube 28 (FIG. 66A), which passes between the reservoir and the pump; hence, the controller displays a tube-kinked alert (or a more general blockage alert) at an alerting step 204. Otherwise, the controller checks, at another checking step 206, whether the expected amount of urine flowed into the reservoir. If yes, the controller returns to the initial sampling step 110. Otherwise, the urinary catheter (or the conduit upstream from the reservoir) is likely clogged; hence, the controller displays a catheter-clogged alert (or a more general blockage alert) at another alerting step 208. Optionally, prior to generating the alert, the controller may raise the pressure in the conduit upstream from the pump, e.g., as described above with reference to FIG. 26, in case the cessation of flow is due to suction acting on the bladder. If the flow subsequently resumes, the alert may be omitted.
(ii) In a second series of steps, the controller calculates various flow parameters, and also checks whether the urine-collection bag is almost full.

First, the controller performs recording step 116, as described above with reference to FIG. 18. Subsequently, at a calculating step 210, the controller calculates the volume of urine that flowed through the pump and the flow rate, along with the accumulated volume of urine in the collection bag. The controller then displays one or more of these parameters at a displaying step 212. In addition, the controller checks, at another checking step 214, whether the collection bag is almost full. If yes, the controller displays an appropriate alert at another alerting step 216.
(iii) In a third series of steps, the controller checks the amplitude of the electrical current driving the pump, at another checking step 218. If the amplitude is higher than a predetermined threshold, it is likely that the urine-collection bag is full, such that the pump is encountering greater resistance; hence, the controller displays an appropriate alert at another alerting step 220.

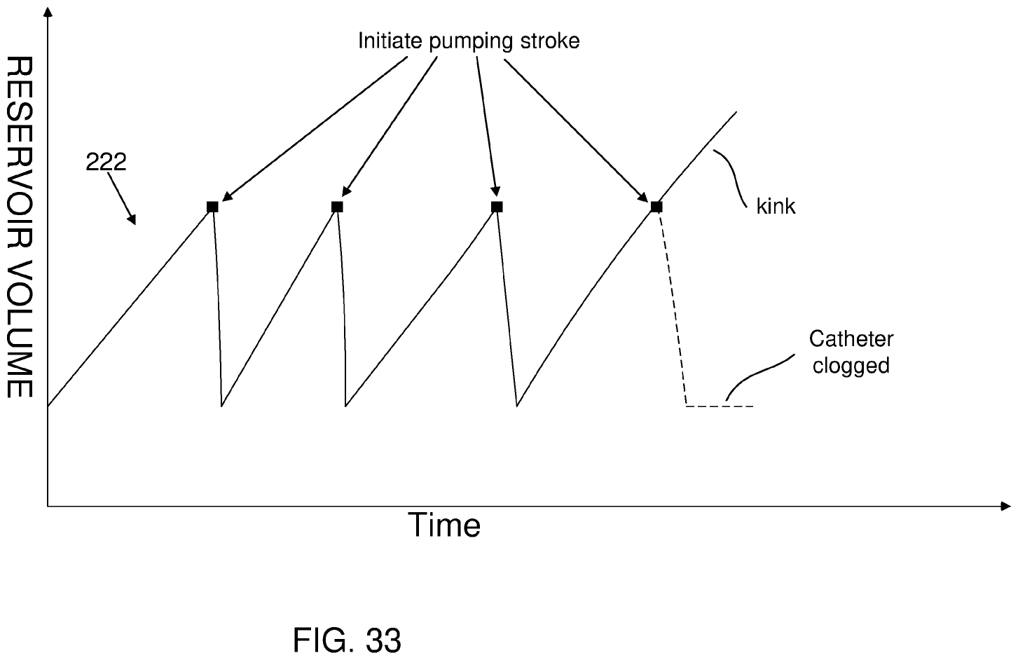

Reference is now made to FIG. 33, which shows a plot 222 tracking a reservoir volume over time, in accordance with some embodiments of the present invention.

Plot 222 is similar to the plot of FIG. 19, in that the plot shows points in time at which pump strokes are initiated. However, plot 222 also shows two cases in which a problem may be detected as described above with reference to FIG. 32: a first case in which there is a kink in the tube, such that no urine is pumped from the reservoir and the reservoir continues filling, and a second case in which the catheter is clogged, such that the reservoir does not refill.

### Noise Filtering and Display of Output

Reference is now made to FIG. 49, which is a schematic illustration of a system 246 for displaying urine-production parameters, in accordance with some embodiments of the present invention.

System 246 comprises a urine-production measuring system 248 configured to measure the amount of urine produced by a subject over time and to compute the rate at which the urine is produced as a function of time. In some embodiments, urine-production measuring system 248 comprises, or is connected to urinary catheter 124 via, tube 28 and connector 72. In some embodiments, urine-production measuring system 248 comprises one or more components of system 96, such as pump 20 and/or controller 125 (FIG. 66B).

In general, urine-production measurements may be distorted due to many factors. Such factors may include, for example, cardiac and respiratory activity of the subject, intestinal motion, body motion, and blockages of catheter 124, e.g., by tissue of the bladder. Hence, the output of urine-production measuring system 248 is a superposition of a clean signal, representing the true rate of urine production as a function of time, and additive noise. In other words, the signal representing the rate of urine production by the kidneys of the subject as a function of time, as computed by the urine-production measuring system, is a noisy signal.

To address this challenge, system 246 further comprises a filtering module 250, which is configured to receive the noisy output signal from the urine-production measuring system and to filter noise from the noisy signal so as to obtain a clean signal. In some embodiments, given that the noise is generally at a higher frequency than the clean signal, the execution of filtering module 250 includes the application of a low-pass filter to the noisy signal. Alternatively or additionally, filtering module 250 may include a neural network trained to filter out the noise.

In some embodiments, the filtering module is executed by the urine-production measuring system, e.g., by controller 125 (FIG. 66B). In other embodiments, as assumed in FIG. 49 and below, filtering module 250 is executed by a processor external to urine-production measuring system 248, such as processor 127 (FIG. 20).

As further described below with reference to FIG. 56, after filtering the signal, the filtering module, or another module executed by the processor, may compute a representative rate of change in the clean signal over at least 12 hours. Subsequently, the processor may generate an output, including a graphical output for example, indicating the representative rate of change. (Typically, as described below with reference to FIG. 56, the processor displays a plot of the clean signal, and marks the representative rate of change on this plot.) The processor may then display the output on a patient monitor 252 and/or another display 254 (e.g., display 378 (FIG. 66B)), and/or communicate the output to an EMR 256, a gateway, a nurse station monitor, and/or a device such as a cellphone or tablet.

Reference is now additionally made to FIG. 56, which is a schematic illustration of displayed output 320, in accordance with some embodiments of the present invention.

In general, given that the rate of urine production may fluctuate due to various factors (related, for example, to the administration of medication or fluids), it may be difficult to manually identify trends in the rate. To address this challenge, the filtering module (or another module) may compute a representative rate of change in the rate, e.g., by applying linear regression or a trained neural network to the clean signal representing the rate.

By way of example, FIG. 56 shows a plot 322 of a subject's urine-production rate over several days. From day 4 to day 8 there is a declining trend in urine production that may be difficult to identify manually. For example, point 326 on day 5 is higher than point 324 on day 4, point 328 on day 6 has approximately the same value as point 324, and point 330, several hours later, is higher than point 324. However, a trend line 332, which may be computed and displayed by the processor, clearly indicates a negative representative rate of change of approximately −50 ml/h/day.

In some embodiments, the processor is further configured to generate an alert in response to the magnitude (i.e., absolute value) of the representative rate of change exceeding a predefined threshold. For example, the processor may generate an alert in response to the representative rate of change being greater than R1 ml/h/h or less than −R2 ml/h/h, where R1 and R2 are positive and are optionally equal to one another.

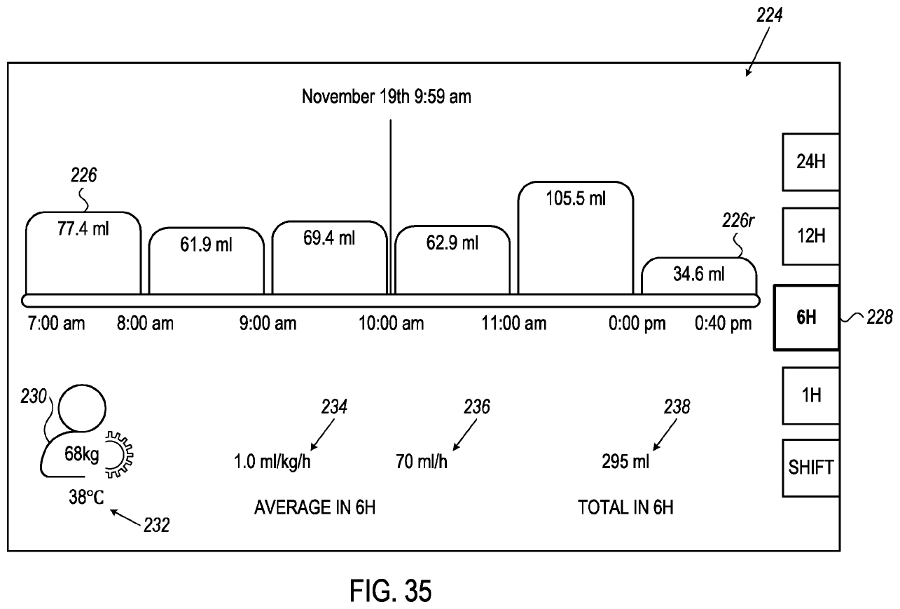

Reference is now made to FIG. 35, which is a schematic illustration of example output 224 that may be displayed on display 378 (FIG. 66B) and/or any other display (such as a patient monitor or a cellphone or tablet display) by the controller, in accordance with some embodiments of the present invention.

In this example, bars 226 indicate hourly volumes of urine production for a preceding period of time that may be selected by the user by clicking the appropriate tab 228. (The rightmost bar 226r shows the volume produced from the start of the current hour.) For example, by selecting the six-hour (6H) tab, the user may see the hourly volumes for the previous six hours. Alternatively, the user may select the preceding one hour (1H), 12 hours (12H), 24 hours (24H), or the period of time from the start of the current shift. In some embodiments, if the duration of the selected preceding period of time is one hour or less, the volumes are displayed with five-minute resolution, i.e., each bar shows the volume of urine production over five minutes.

Optionally, output 224 may further include an indicator 230 of the subject's weight, which may be entered by a nurse, for example. Alternatively or additionally, the display may include an indicator 232 of the subject's core body temperature, which may be measured, for example, as described below with reference to FIG. 40. Alternatively or additionally, for the selected preceding time period, output 224 may include an average per-weight rate 234 of urine production (e.g., in units of ml/kg/h), an average rate 236 of urine production, and/or a total volume 238 of urine production. In some embodiments, output 224 includes a total volume of urine production over the previous 60 minutes, regardless of which tab is selected.

### Bag Connector for Replaceable Fluid Bag

As described above with reference to FIG. 22, in some embodiments, exit tube 29 is connected to urine-collection bag 78 via connector 134, bag connector 136, and connecting tube 135. In such embodiments, when urine-collection bag 78 fills up, it can be replaced with a new bag without the need for replacing the entire disposable kit and without the need to empty the bag.

More generally, this type of connection may be used with any replaceable fluid bag; hence, the more general term “fluid bag” or “replaceable fluid bag” is used below instead of “urine-collection bag.”

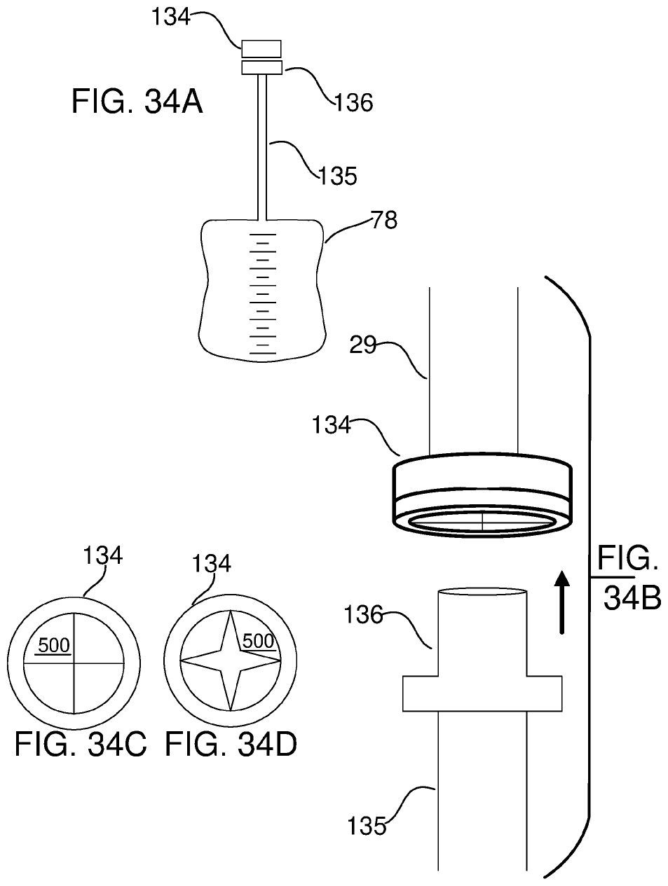

In this regard, reference is now made to FIG. 34A, which is a schematic side view of replaceable fluid bag 78 with spill-proof connector 134, in accordance with some embodiments of the present invention. Reference is further made to FIG. 34B, which is a schematic detail view of the spill-proof connector of FIG. 34A. Reference is further made to FIGS. 34C and 34D, which are schematic frontal views of spill-proof connector 134 in closed and open configurations, respectively.

Connector 134 in this embodiment is a non-spill female connector, which is fixed to the downstream end of exit tube 29, which receives a fluid output by a subject (such as urine) through its upstream end. Connector 134 comprises multiple flexible leaves 500, which close together across the connector to prevent outflow of the fluid, as shown in FIG. 34C. Leaves 500 comprise sections (e.g., quadrants) of a polymer diaphragm, made from rubber or silicone, that extends across connector 134. Bag connector 136 is a male connector, which is inserted into connector 134 and thus opens the flexible leaves by pushing the leaves inward, as shown in FIG. 34D. The fluid can then flow out of exit tube 29 and through connecting tube 135 into fluid bag 78, which is fixed to bag connector 136 (via connecting tube 135) so as to receive and store the fluid flowing out of the exit tube.

In other embodiments, connector 134 is male and connector 136 is female.

In yet other embodiments, connector 134 and connector 136 are genderless. For example, a lock, comprising snaps for example, may lock the connectors together. Optionally, in such embodiments, the connectors may be sealed to one another via an O-ring seal.

In some embodiments, the connectors are coupled to one another by pushing one connector toward the other. In other embodiments, the connectors are coupled to one another by turning one connector relative to the other (e.g., by a one-quarter turn).

In alternate embodiments, connector 134 is not a non-spill connector.

Catheter-Tube Connector with Temperature Sensor

FIG. 40 is a schematic side view of catheter connector 72 with an integral temperature sensor 505, in accordance with some embodiments of the present invention.

Connector 72 has an upstream end 502 for connection to a urinary catheter. A tube 504 is connected to the downstream end of connector 72 so as to receive urine flowing through the catheter. (An example of tube 504 is tube 28 per FIG. 30.) A temperature sensor 505 is functionally associated with connector 72. For example, temperature sensor 505 may be integral with the connector, as in FIG. 40. Alternatively, the temperature sensor may be near the connector, e.g., by virtue of being integral with a sampling port coupled to the connector or by virtue of being disposed in a separate housing coupled to the connector directly or via a short tube.

Temperature sensor 505 senses the temperature of the urine flowing into tube 504. In the pictured embodiment, temperature sensor 505 comprises an electrical sensor, such as a thermocouple, which is contained within connector 72 and outputs an electrical signal that is indicative of the temperature of the urine. Alternatively, other types of temperature sensors may be used, and the temperature sensor may be either within the connector or at a location outside the connector.

A wire 506 is connected to temperature sensor 505 and extends along tube 504 so as to convey the electrical signal to a measurement circuit, such as a monitor or another device for displaying and/or recording the subject's temperature (not shown in this figure). Wire 506 may be integral with tube 504, for instance by passing through the wall of the tube or through a lumen of the tube and terminating at the downstream end of the tube.

Upstream end 502 of catheter connector 72 plugs into urinary catheter 124 (FIG. 37), such as a Foley catheter. Connector 72 may have a sampling port 372 for taking urine samples. Sensor 505 may comprise a thermocouple, as noted above, or alternatively a thermistor or other resistance temperature detector (RTD), or other means for temperature measurement.

In some embodiments of the present invention, the end of tube 504, together with wire 506, may be connected to urine-production measuring system 248 (FIG. 49), which measures the urine flow. The measuring system may also comprise circuitry to estimate the temperature sensed by temperature sensor 505 in connector 72. The measuring system may display the temperature and/or transmit it to a hospital electronic medical record (EMR), a patient monitor, a nurse station monitor, and/or a gateway. Since the measuring system knows the urine flow-rate at each moment, it can sample the temperature sensor at times when the flow is above a certain rate, thus making sure that the urine that came out from the bladder did not cool before it reached the sensor. If the measuring system is associated with means for controlling the flow (as in some embodiments of this invention, such as embodiments in which the urine is pumped), then when the subject's urine production is very low, the measuring system can stop the urine flow out of the bladder (e.g., by stopping the pump) for a short period in order for urine to accumulate in the bladder and can then let the urine flow out at a high rate and sample the temperature sensor when the warm urine from the bladder reaches connector 72.

In an alternative embodiment, the temperature sensor comprises a capillary tube, which extends along tube 504 and is connected to a pressure measurement device at the downstream end of the tube. The pressure in the capillary tube varies with the temperature; hence, the pressure measurement device may measure (or “estimate”) the temperature indirectly, by measuring the pressure in the capillary tube. Thus, the temperature can also be measured in a non-electrical manner. This embodiment can be implemented using a dual-lumen tube (as shown in FIG. 15, for example), with one lumen for the urine and the other lumen for pressure changes caused by temperature changes. The pressure is then measured at the downstream end of the tube and translated into a temperature, which is displayed and/or registered on a monitor and/or another measurement or display device. Pressure-based temperature estimation can be carried out, for instance, using a sealed bellows or bulb along with capillary elements, which are filled with a gas or liquid that expands or contracts in response to the temperature.

### Spring-Loaded Safety Release for a Peristaltic Pump

FIGS. 36A and 36B are schematic side views of peristaltic pump 20 with a spring-loaded safety release in normal and released configurations, respectively, in accordance with some embodiments of the present invention. Pump 20 comprises a pumping mechanism, such as rotor 22 comprising rollers 24, which propels fluid through a flexible part of peristaltic pump tube 33. (Typically, although not necessarily, the entire length of pump tube 33 is flexible.) Alternatively, the principles of this embodiment may be applied in conjunction with other sorts of pumping mechanisms and tube configurations.

FIGS. 36A and 36B show an example of a mechanism for releasing the grip of pump 20 on pump tube 33. In normal operation, rollers 24 roll and press against a part of pump tube 33, while clamp 26 presses the part of the pump tube against the rollers, so that the rollers compress the pump tube. If there is some system failure (e.g., a pump actuator failure, software bug, or power failure) that causes rotor 22 to be unable to turn, however, the urine flow will be blocked since clamp 26 in pump 20 keeps pressing pump tube 33 against rollers 24 of rotor 22. Therefore, pump 20 comprises a release mechanism, which receives an indication of a malfunction in a fluid circuit to which pump tube 33 is connected and, in response to the indication, releases clamp 26 so that urine will be able to flow freely through the pump tube.

In the present example, the indication of the malfunction comprises an increase in a pressure in a section 518 of pump tube 33 at a location upstream of pump 20. The release mechanism comprises a moveable rod 516, having one end in contact with section 518 of pump tube 33, so that the increase in pressure causes the tube to swell and move the rod, which releases clamp 26. In normal operation, a spring 511 applies compression against clamp 26 so as to press the clamp against pump tube 33. The release mechanism releases the compression in spring 511 in response to the malfunction indication.

More specifically, a rod 512 pushes against a spring 511, which pushes clamp 26 against pump tube 33. Rod 512 is normally held in place by a rod 513, which in turn pushes against a spring 521 and is held in place by rod 516. Rod 516 is held against pump tube 33 by a spring 515. Section 518 of pump tube 33 has a thinner wall than the rest of the tube. When a malfunction occurs, urine backup will cause the pressure in pump tube 33 to increase, and section 518 will start to inflate, pushing rod 516 against spring 515 until rod 513 retracts into a notch 522 by spring 521. Retraction of rod 513 releases the hold of rod 512, which is then pushed back by the expansion of spring 511, thus causing clamp 26 to release its pressure on pump tube 33 and allowing urine to flow freely through the tube.

Additionally or alternatively, an electromechanical switch, such as a solenoid 517, can be used to release clamp 26 in response to an electrical signal that is indicative of a malfunction. Solenoid 517 can be actuated by software, by a manual command, by power failure, or by action of a safety sensor. The plunger of solenoid 517 is out, as shown in FIG. 36B, unless power is applied to the solenoid as shown in FIG. 36A. During normal operation of pump 20, power is applied to solenoid 517, which pulls the plunger in. When there is a failure, power is cut off to the solenoid, which causes the plunger to push rod 516, which will then release rod 512 as described above.

### Pressure Control Using Spring-Loaded Components in a Peristaltic Pump

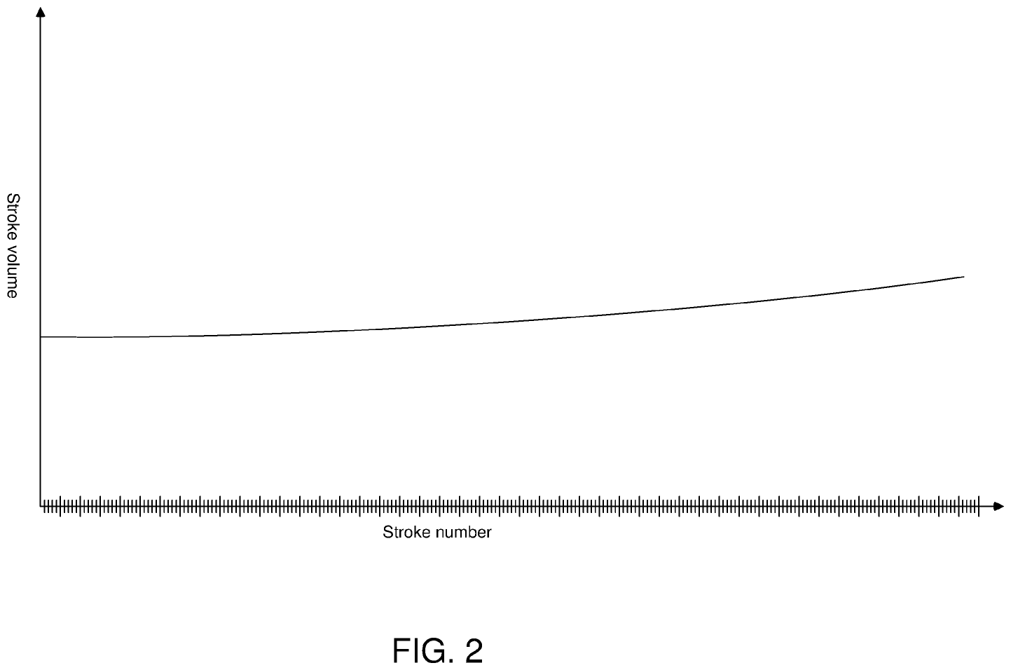

FIG. 2 is a plot showing an example of an aging graph of peristaltic pump tube 33 (FIGS. 1B-C). The graph shows the change in stroke volume of a peristaltic pump as a function of the number of strokes applied to the pump tube in the pump. As can be seen from the graph, the stroke volume tends to increase as the pump tube wears out.

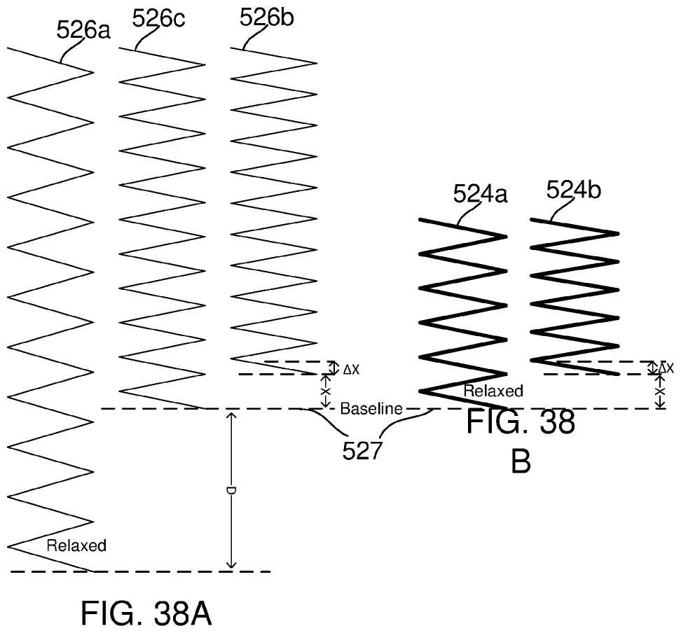

FIGS. 38A and 38B are schematic side views of springs 526 and 524, which are used in controlling pressure exerted by a clamp in a peristaltic pump (such as clamp 26 in pump 20), in accordance with some embodiments of the present invention. Spring 526 has a spring constant k1, while spring 524 has a spring constant k2. The springs are labeled 526a and 524a, respectively, in their relaxed (not squeezed) state, and they are labeled 526b and 524b when squeezed a distance X from a given baseline 527.

The force applied by a spring is calculated according to Hooke's law as F=k*X, wherein F is the force, k is the characteristic (spring constant) of the spring, and X is the displacement of the edge of the spring. In order for springs 524 and 526 with different characteristics to exert the same force when displaced the same distance X, the weaker spring should be biased (pre-squeezed from its relaxed state). This sort of biased state of spring 526 is labeled 526c, with a bias offset of D. In this case F1=k1*(D+X), and F2=k2*X. To satisfy F1=F2, the offset should be chosen so that k1*(D+X)=k2*X. When D>>X, this condition will be satisfied for k2>>k1.

If both springs are compressed from their squeezed states 524b and 526b by an additional distance ΔX, then the additional force applied by each spring will be k*ΔX. For spring 526, the additional force will be k1*ΔX, while for spring 524, the additional force will be k2*ΔX. Since k1<<k2, the additional force applied by spring 526 as a result of the additional movement ΔX will be much smaller than the additional force applied by spring 524 as a result of the same additional movement ΔX. In other words, the change in force applied by a spring with a small k as a result of squeezing the spring by a certain additional amount will be much less than the change in force of a spring with a large k that is squeezed by the same additional amount. By using a long spring with a small k, the force applied by the spring will remain nearly constant for small displacements of the spring.

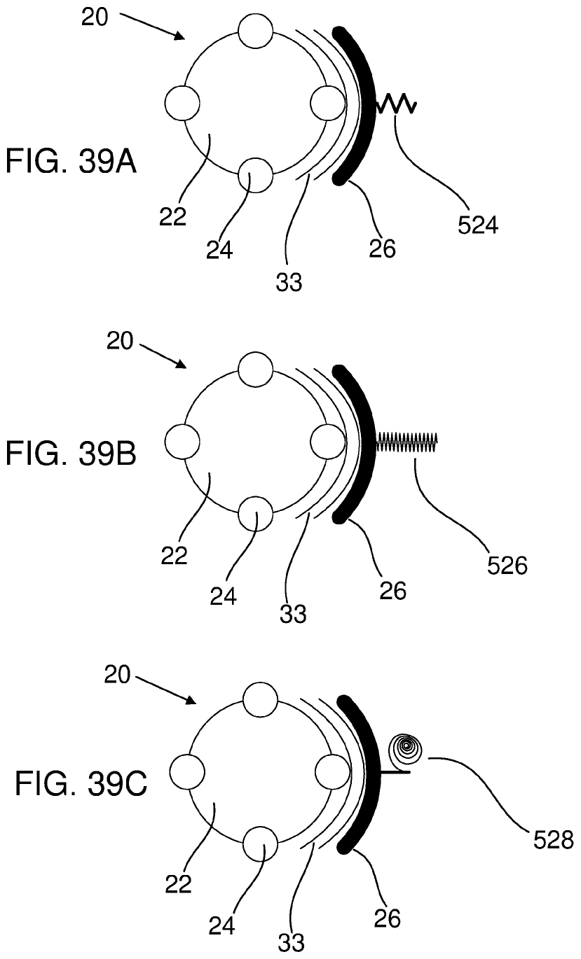

FIGS. 39A, 39B and 39C are schematic side views of peristaltic pumps 20 with spring-loaded pressure clamps 26, in accordance with embodiments of the invention. Each pump 20 comprises a rotor 22, comprising multiple pressing elements in the form of rollers 24 (for example, four rollers), which roll and press against a part of flexible pump tube 33, through which a fluid flows from a fluid source, such as a urinary catheter. One or more springs 524, 526, 528 apply a compression (i.e., a compressive force) between rollers 24 and clamp 26 so that the rollers apply a force against pump tube 33 that remains substantially constant irrespective of variations in the hardness or thickness of the tube. (In the context of this embodiment and in the claims, the term “substantially constant” means that the force remains within ±5% of its initial value.)

Specifically, in the embodiments shown in FIGS. 39A-C, springs 524, 526, and 528 are attached to clamp 26. (Alternatively, in the embodiments that follow, springs are coupled to the rollers or to the rotor.) Springs 524 and 526 are linear springs, with high and low spring constants, respectively, as explained above. Spring 528 is a coiled spring, i.e., a spiral-wound torsion spring (also referred to as a rotor spring). An advantage of this type of spring is that it is small in its outer dimensions, yet can be very long.

According to some embodiments of the present invention, pump tube 33 is disposable; for example, the pump tube may belong to disposable kit 370 (FIG. 66A). In addition, clamp 26 may be disposable. Hence, the pump is used with multiple different pump tubes and, optionally, multiple different clamps. Since there may be tolerances in the dimensions of these elements during manufacturing, the force applied by the clamp on the pump tube may vary, because the spring will be pressed a different amount due to variations in the pump tube and/or clamp dimensions.

To avoid this variation in force, which can lead to variation in the stroke volume of the pump, the spring should be chosen to exert a substantially constant force over a range of displacements. For example, a long spring with low spring constant, such as spring 526, or a spiral spring, such as spring 528, each of which is biased, i.e., squeezed significantly relative to the working displacement, could be used for this purpose. As a result, small differences in the clamp and pump-tube geometry will practically not affect the force applied by the spring, since the variance in the spring displacement as a result of these differences will be much smaller than the biasing displacement (as explained with reference to FIG. 38). By the same token, the force applied by the spring on the pump tube changes very little as the pump tube wears out, thus maintaining a uniform stroke volume and prolonging the life of the pump tube.

The embodiments that are described below relate to rotational peristaltic pumps, in which rollers 24 press against and compress a part of a flexible pump tube in order to propel fluid through the pump tube. In these embodiments, one or more springs apply a compression between the pressing elements, i.e., rotor 22 comprising rollers 24, and clamp 26 so that the pressing elements apply a force against the part of the flexible pump tube such that the force remains substantially constant irrespective of variations in mechanical characteristics of components of the pump, for example due to wear of the pump tube. Alternatively, the principles of these embodiments may be applied, mutatis mutandis, to peristaltic pumps of other types, with other sorts of pressing elements. For example, these principles may be applied in enhancing the performance of linear peristaltic pumps, in which the pressing elements comprise linear translational elements (“fingers”), which press sequentially against a flexible pump tube.

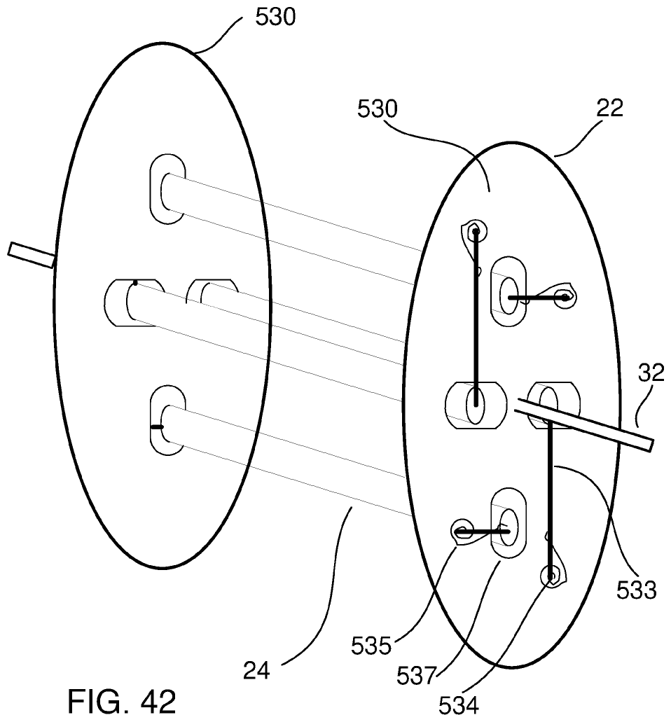

FIGS. 41 and 42 schematically illustrate rotor 22 of a peristaltic pump with spring-loaded rollers 24, in accordance with embodiments of the invention. FIG. 41 is a side view, while FIG. 42 is a pictorial view. In both of these embodiments, rotor 22 comprises a rotating drum 530, in which rollers 24 are mounted. Springs 535 are coupled to shift the rollers radially outward within drum 530, thus maintaining a substantially constant force between the rollers and pump tube 33 (not shown in these figures). Specifically, rollers 24 are mounted on respective rods 533, which pivot about respective axes 534 on drum 530. Springs 535 are attached to rods 533 and exert a rotational force on the rods about the respective axes.

In the pictured examples, springs 535 are stretched counterclockwise around axes 534 and push rods 533 clockwise around the axes, thus pushing rollers 24 outward relative to axle 32 at the center of rotor 22. The force that springs 535 apply causes rollers 24 to squeeze pump tube 33 against clamp 26. Springs 535 may be designed to apply a force that increases with displacement, in accordance with Hooke's law, or they may be designed to exert a substantially constant force, as explained above, for overcoming manufacturing tolerances. Alternatively, the springs may comprise a constant-force spring for adjusting the force of a Hooke's-law spring so as to reduce sensitivity to tolerances of the pump components (such as the clamp, pump tube, and rotor).

In FIG. 41, a pin 536 serves as a stopper for limiting the movement of rods 533 when there is no pump tube in the pump and roller 24 is not pressing on the pump tube.

In FIG. 42, rollers 24 are mounted within respective radial slots 537 in drum 530. The tension applied by springs 535 shifts the rollers radially within the radial slots, while the bounds of the slots limit the movement of the rollers.

In the embodiments of FIGS. 41 and 42, drum 530 has a larger diameter than the diameter of a circle 538 where rollers 24 are disposed. This difference in diameters leaves room on drum 530 for springs 535 and rods 533 to be placed outside the radius of circle 538.

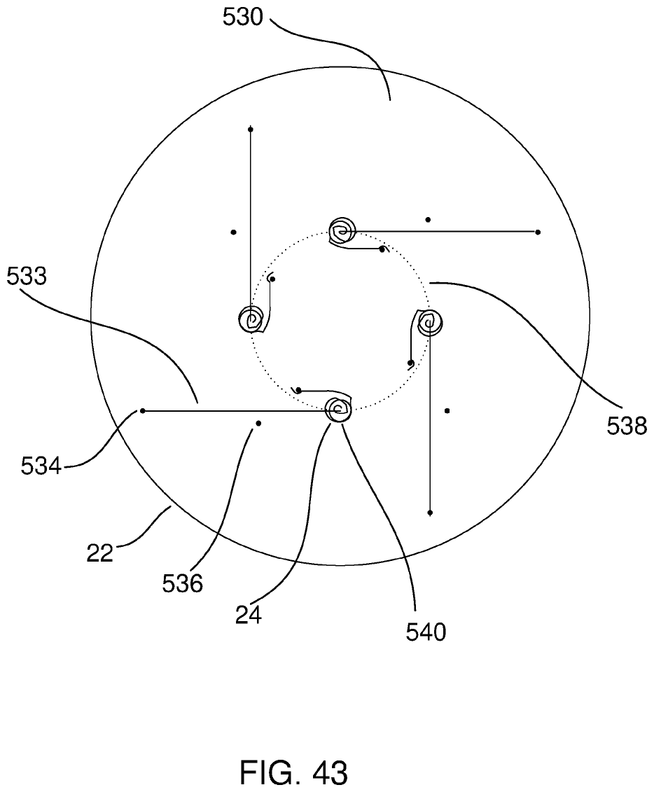

FIG. 43 is a schematic side view of rotor 22 of a peristaltic pump with spring-loaded rollers 24, in accordance with yet another embodiment of the invention. This embodiment is similar to the embodiments of FIGS. 41 and 42, except that in the present embodiment, springs 540 are attached to rollers 24. Rollers 24 are attached to the end of rods 533 as in the preceding embodiments, but springs 540 push the rollers directly rather than pushing the rods. Thus, rods 533 serve only to hold and guide the rollers in their travel. This arrangement is advantageous in that smaller springs can be used since smaller force is needed.

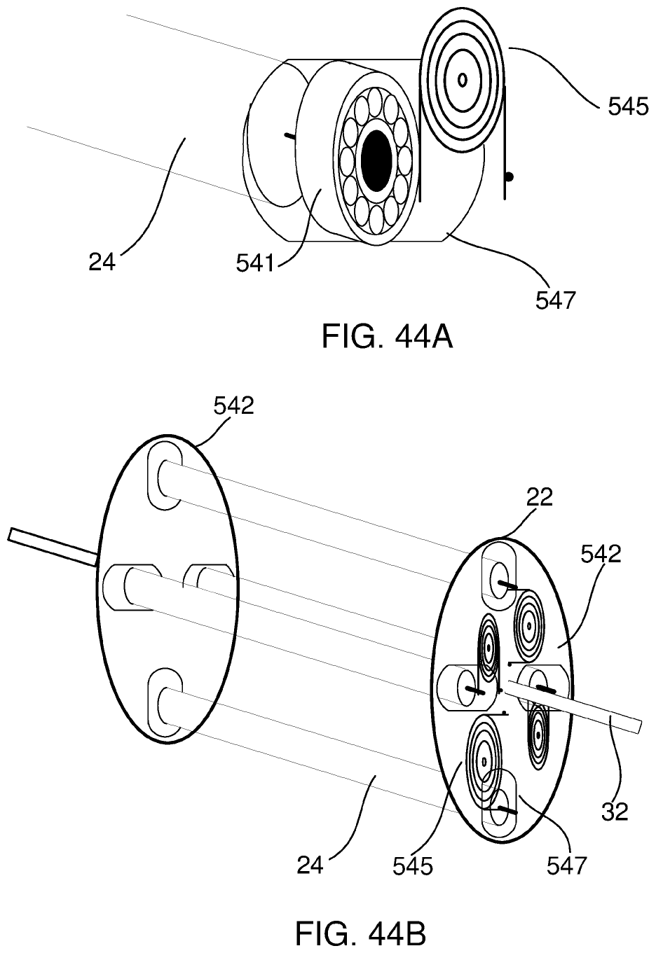

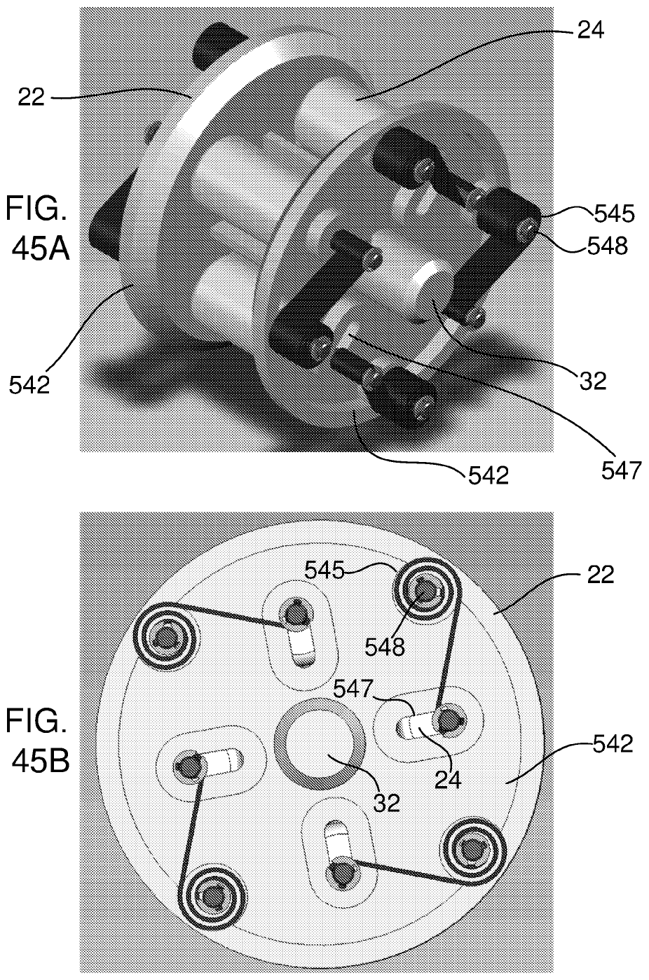

Reference is now made to FIGS. 44A and 44B, which schematically illustrate rotor 22 of a peristaltic pump with spring-loaded rollers 24, in accordance with a further embodiment of the invention. FIG. 44A is a schematic detail view of one of rollers 24 with a spring-loaded rotational bearing 541, while FIG. 44B is a schematic pictorial view of the entire rotor 22.

In this example, rollers 24 comprise rotational bearings 541, which are connected to the ends of the rollers and slide radially within radial slots 547 in a drum 542, which limits the motion of the rollers. Springs 545 push the rollers radially outward. As in the preceding embodiments, springs 545 may be designed to apply a force that increases with displacement, in accordance with Hooke's law, or they may be designed to exert a substantially constant force, or they may comprise a constant-force spring for adjusting the force of a Hooke's-law spring. Bearings 541 are useful in reducing friction so that the roller will roll smoothly against the pump tube, thus reducing pump-tube wear. In this example, the diameter of drum 542 is only slightly larger than the distance between the far edges of two opposite rollers.

FIGS. 45A and 45B are schematic pictorial and side views, respectively, of spring-loaded rollers 24 of a peristaltic pump, in accordance with still another embodiment of the invention. As in the preceding embodiment, rollers 24 are mounted to shift radially within slots 547. Springs 545, mounted on respective axes 548, apply a force to drive rollers radially outward against a flexible pump tube. In this embodiment, drum 542 has a larger diameter than in the preceding embodiment in order to accommodate the longer springs.

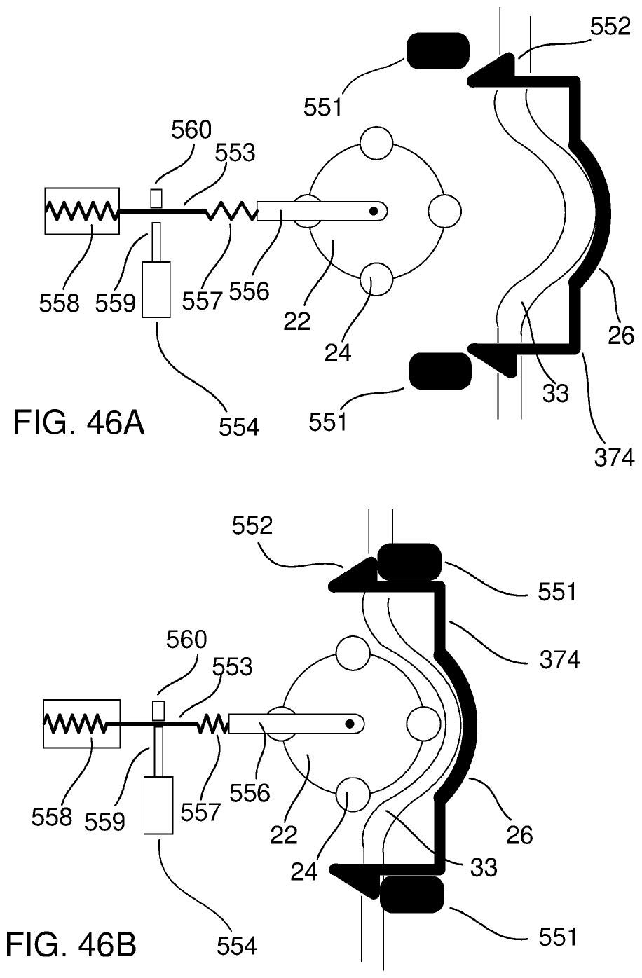

FIGS. 46A and 46B are schematic side views of a peristaltic pump with a replaceable (i.e., disposable) cartridge 374 before and after attachment of the cartridge to the pump, in accordance with some embodiments of the present invention. Cartridge 374 includes pump tube 33, clamp 26, and a latch 552 for holding the cartridge in place when it is inserted into the pump. Springs 557 and 558 are coupled to press rotor 22 toward clamp 26.

Since cartridge 374 is replaceable and has a manufacturing tolerance, the pump cannot be pre-calibrated for any specific cartridge, and there is thus a need for a mechanism that will be able to tolerate these tolerances for achieving high precision pumping volumes. In addition, pump components, such as the rotor, the rollers, the bearings, and the clamp, wear during operation, and there is a need to accommodate this wear to maintain high accuracy.

In the present example, rotor 22 is attached by a strut 556 to a spring 557, which pushes the rotor to the right. Spring 557 in turn is pushed by a constant-force spring 558 through a rod 553. When cartridge 374 is initially plugged into the pump, rotor 22 is turned to a predefined position (for example, the position shown in these figures). Cartridge 374 is pushed toward the left, causing spring 557 to compress until it reaches the force of spring 558. At this point, spring 558 will start to compress, while spring 557 will not compress any further in view of the approximately constant force applied by spring 558.

When cartridge 374 has been fully inserted (moving to the left), latches 552 will snap into place against stoppers 551. In this position, with spring 557 squeezed at the constant force of spring 558, a solenoid 554 pushes a plunger 559 against rod 553 to hold the rod in place against a stopper 560. Thus, solenoid 554 and plunger 559 serve as a lock, which opens during insertion of cartridge 374 into the pump in order to permit springs 557 and 558 to drive rotor 22 toward clamp 26 to a location at which rollers 24 apply the desired constant force against flexible pump tube 33. Solenoid 554 and plunger 559 then lock the end of spring 557 that is farther from the rotor in this location during operation of the pump. When the pump starts running and rotor 22 turns, spring 557 is anchored by rod 553 on its left side and pushes rotor 22 to squeeze pump tube 33 against clamp 26 on its right side.

This mechanism reduces the sensitivity of the pump to tolerances in the dimensions of cartridge 374 since at the time of insertion of the cartridge, spring 557 is squeezed with the force of spring 558, which is substantially constant, rather than with a force that is a function of the thickness of clamp 26 or of the pump tube, for example, as would be the case if spring 557 were simply anchored to the body of the pump. In an alternative embodiment, a similar arrangement of springs can be used to push the cartridge toward the rotor, while the rotor is held in a fixed position. In still another embodiment, the cartridge and the rotor are fixed, and the rollers of the rotor are pushed by this sort of combination of springs with a lock.

In all of these embodiments, when the cartridge is inserted into the pump, the rotor or the cartridge or the rollers are brought to a known, predefined position. Constant-force spring 558 plays a role while the cartridge is inserted in order to compensate for any tolerances in the dimensions of the cartridge components and clamp so that spring 557 will exert the same force regardless of the cartridge dimensions. Once the cartridge is inserted, solenoid 554 locks the position of spring 557 in place. Thus, spring 557 causes rollers 24 and clamp 26 to apply the same force on pump tube 33 regardless of dimensional variations of the clamp and the cartridge component dimensions. As spring 557 behaves according to Hooke's law, this arrangement ensures that the initial force on pump tube 33 will be fixed regardless of mechanical tolerances, but will change, for example, as the pump tube wears.

In an alternative embodiment, spring 557 and solenoid 554 are omitted, and only spring 558 applies force against rotor 22 or clamp 26. In this arrangement, too, the force applied on pump tube 33 is constant regardless of mechanical tolerances and wear.

### Hanging Scale for Fluid Bag

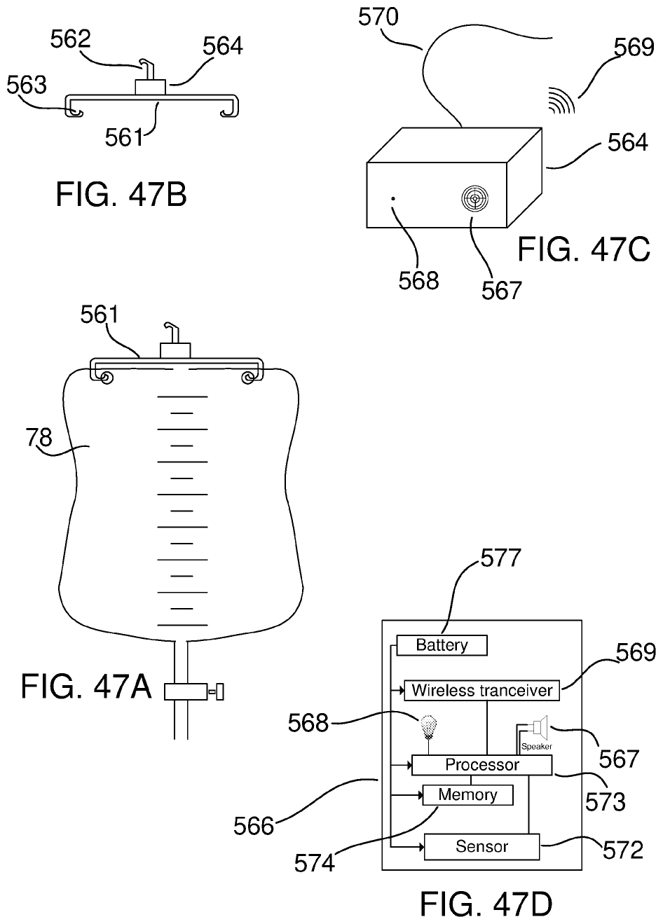

Reference is now made to FIGS. 47A-D, which schematically illustrate a hanging scale 561 for a fluid bag that receives fluid excreted from a body of a subject, such as urine-collection bag 78, in accordance with some embodiments of the present invention. FIG. 47A is a side view of hanging scale 561 and bag 78, while FIG. 47B is a detail view of scale 561. FIG. 47C is a detail view of a controller 564 that is integrated into hanging scale 561, and FIG. 47D is a block diagram that schematically illustrates circuitry 566 in controller 564. Scale 561 comprises a hanger 562, having hooks 563 from which urine-collection bag 78 is suspended. Hanger 562 has a hook for hanging or otherwise attaching hanging scale 561 to a support, such as an IV pole, a subject's bed, an infusion pump, urine-production measuring system 248 (FIG. 49), or the wall. A sensor 572 senses a quantity of a fluid in bag 78, which is indicative of the quantity of fluid excreted by the subject.

A similar arrangement to that shown in FIGS. 47A-D can be used to hang and measure the quantity of fluid in other sorts of fluid bags, such as an intravenous infusion bag. In this case, sensor 572 is used to sense the quantity of fluid delivered to the subject.

Hanging scale 561 includes a controller 564 comprising electronic circuitry 566, including sensor 572 for detecting the fill level of the bag. In some embodiments, sensor 572 measures the weight of the fluid in bag 78. For this purpose, sensor 572 may comprise, for example, a strain gauge, a load cell, or a spring combined with a detector such as a potentiometer, an optical detector, a variable capacitor, or a variable inductor. Additionally or alternatively, sensor 572 measures the level of the fluid in bag 78, for example using an ultrasonic or optical detector to detect the liquid surface level. As another option, sensor 572 may detect the inflation and deflation of bag 78 in order to determine the quantity of fluid in the bag. This approach is advantageous in that it is not influenced by the weight of the bag itself and by strain on the tube, which may affect the weight measurement. Controller 564 may measure the quantity of fluid in bag 78 using any of the above methods individually or in combination, as well as using other methods that will be apparent to those skilled in the art after reading the present description.

Controller 564 issues an alarm when the quantity of fluid in bag 78 reaches a predefined limit (for example when bag 78 is almost full or almost empty as the case may be). For this purpose, controller 564 may include an audible alarm 567 and/or a light 568. The functions of controller 564 are coordinated by a processor 573 with a memory 574. A battery 577, which may be rechargeable or non-rechargeable, provides power to these and the other elements of circuitry 566. Alternatively or additionally, circuitry 566 may receive power from the mains. Processor 573 handles functions such as communications, control calibration and zeroing of sensor 572, receiving readings from the sensor, calculating and determining whether the quantity of fluid in the bag has reached a threshold, and operating the audible and visual alarms. Memory 574 stores the program code, data, configuration data, and historical data, for example.

In the pictured embodiment, controller 564 comprises a communication link, such as a wireless transmitter or transceiver 569 or a wired link 570, to convey an indication of the sensed quantity of the fluid to a receiver. The wireless transmitter or transceiver may operate in accordance with any suitable standard or proprietary protocol, such as Wi-Fi, Bluetooth, Zigbee, or NFC, for example. The communication link can be used to send alarm messages when the quantity of fluid in bag 78 reaches the predefined limit. The communication link may also be used to configure controller 564, such as by setting the alarm configuration and threshold levels. Several threshold levels may be set, such that each threshold will trigger an alarm with a different severity level. Other configurable parameters may include the alarm volume, audio type, and visual type (such as blink speed and intensity), for example.

The communication link may also be used to send historical data, either upon request or periodically. Such history data may include, for example, the number of bags replaced, the time each bag was replaced, the time from alarm to bag replacement, and the fill level when the bag was replaced. Additionally or alternatively, the communication link may be used to alert that battery 577 is low and needs to be recharged or replaced.

The data may be transmitted from hanging scale 561 to a receiver, such as a gateway, which may also communicate with other hanging scales of this sort. The data may be communicated to a monitoring system (comprising, for example, a patient monitor and/or a nurse station monitor) and/or to the EMR, either via the gateway or directly from the hanging scale. Alternatively or additionally, the data may be transmitted to urine-production measuring system 248 (FIG. 49) so as to indicate, to system 248, the fill level of the bag and/or that the fill level reached a threshold. In any of these cases, the gateway, monitoring system, or measuring system receives an indication from hanging scale 561 of the sensed quantity of fluid in bag 78 and/or that the fill level reached a threshold, and is thus able to compute and display information regarding excretion of urine by the subject over time and/or alert that the bag is almost full. Alternatively, when the hanging scale is used to hang an infusion bag, controller 564 may send a signal to an infusion pump, a gateway, a monitoring system, and/or an EMR indicating the fill level of the bag and/or that the bag has emptied to a predetermined threshold. The infusion pump, gateway, monitoring system, and/or EMR is thus able to compute and display information regarding fluid administration to the subject over time.

A monitoring system may receive data from hanging scale 561 via either a wired or a wireless link. The monitoring system may comprise means to alert the medical staff by visual and/or vocal alarm, such as a speaker, buzzer, and/or a lamp. The monitoring system may also have means to input data, such as a keyboard, for setting configuration parameters of hanging scale 561, such as alarm thresholds. The monitoring system may have means to output data, such as a display for displaying historical data, such as the number of bags replaced, total volume of the bags filled (in the case of intravenous infusions, for example) or emptied (in the case of urine excretion, for example), as well as real-time information, such as bag fluid levels. Thus, the monitoring system can display information regarding both excretion of fluid by a subject and input of fluids to the body of the subject over time. These sorts of data can be displayed with respect to multiple subjects concurrently.

The weight of fluid measured by hanging scale 561 may not be stable for several reasons, such as shaking of bag 78 and strain on the bag or on the tube connected to the bag. This instability is referred to herein as “noise.”

In some embodiments, the device receiving indications of fluid quantity from the hanging scale may filter out the noise (e.g., as described with reference to FIG. 49) in order to estimate the actual weight. Alternatively, processor 573 of circuitry 566 of the hanging scale may filter out the noise. The monitoring system, urine-production measuring system, or infusion pump may display the current weight of the bag and/or the current flow rate (i.e., bag fill or empty rate) in real-time and may provide alerts when the flow rate goes above or below predetermined limits and/or when the bag fill level reaches a threshold.

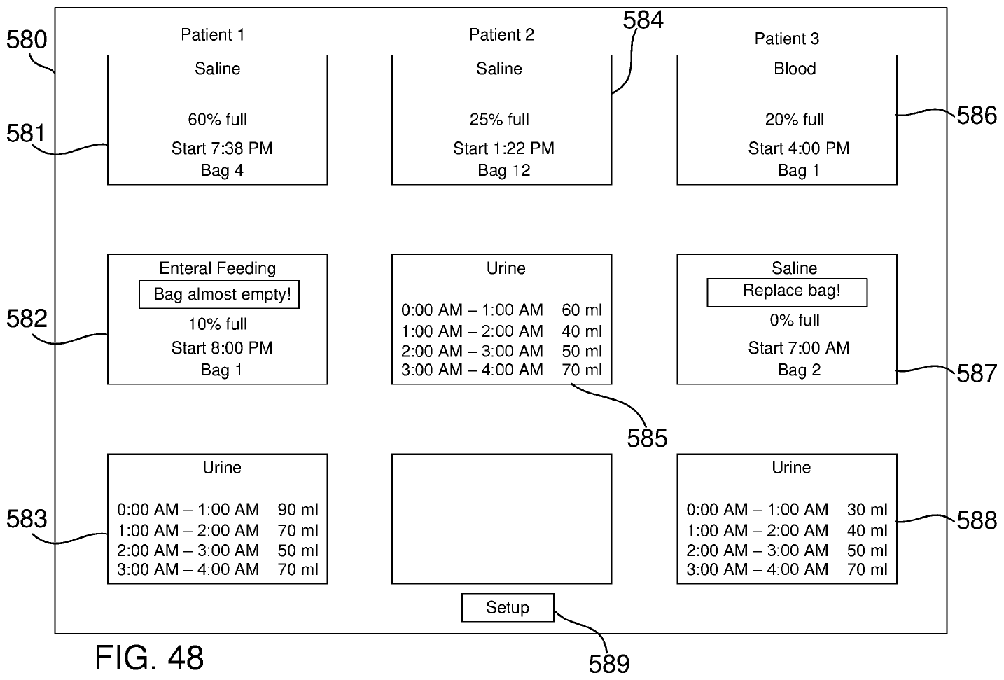

FIG. 48 is a schematic representation of a display screen showing a fluid-management dashboard 580, in accordance with some embodiments of the present invention. Dashboard 580 is displayed by a receiving device, such as a monitoring system, and presents information regarding multiple bags (including both infusion bags and urine-collection bags) that are connected to each of several subjects.

In the present example, dashboard 580 presents information regarding eight bags that are connected to three different subjects. Blocks 581, 582 and 583 display information regarding three bags of subject 1; blocks 584 and 585 display information regarding two bags of subject 2; and blocks 586, 587 and 588 display information regarding three bags of subject 3. Block 581 displays information regarding an IV saline bag connected to subject 1 and hanging from a hanging scale (as shown in FIGS. 47A-D) near subject 1. According to block 581, the fourth bag was replaced at 7:38 PM and is 60% full. Block 582 displays information regarding an enteral feeding bag connected to subject 1 and hanging from another hanging scale near subject 1. The first enteral feeding bag was started at 8:00 PM and is 10% full, and an alert that the bag is almost empty is displayed. Block 583 displays information regarding a urine bag connected to subject 1 and hanging from yet another hanging scale near subject 1. Block 583 shows the hourly urine output for the last four hours. This list can be scrolled to see more historical data.

Regarding subject 2, block 584 displays information regarding an IV saline bag connected to subject 2 and hanging from a hanging scale near subject 2. According to block 584, the twelfth bag was replaced at 1:22 PM and is 25% full. Block 585 displays information regarding a urine bag connected to subject 2 and hanging from a hanging scale near subject 2. The displayed information includes the hourly urine output for the last four hours, and the list can be scrolled to see more historical data.

Regarding subject 3, block 586 displays information regarding an IV blood bag connected to subject 3 and hanging from a hanging scale near subject 3. Block 586 indicates that the first bag was started at 4:00 PM and is 20% full. Block 587 displays information regarding an IV saline bag connected to subject 3 and hanging from a hanging scale near subject 3. Block 587 indicates that the second bag was replaced at 7:00 AM and is empty and displays an alarm calling for the empty bag to be replaced. Block 588 displays information regarding a urine bag connected to subject 3 and hanging from a hanging scale near subject 3. The displayed information includes the hourly urine output for the last four hours, and the list can be scrolled to see more historical data.

Each of the above blocks can be tapped to open a window for displaying additional information or for setup. A setup control 589 can be used to configure parameters such as:
The type of bag (urine, saline, blood, etc.).
The empty or fill threshold level for which a visual alarm or alert will be displayed.
The empty or fill level for which a vocal alarm or alert will sound.
The fill or empty rate above or under which a visual alarm or alert will be displayed.
The fill or empty rate above or under which a vocal alarm or alert will sound.
Enabling and disabling visual and/or audible alarms.
Setting the alarm volume, audio type, and visual type.

Additional information that may be displayed may include historical data, such as the time each bag was replaced and the time from alarm to bag replacement. Other information may include battery level.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a conduit that is configured to carry urine downstream from a bladder of a subject, the apparatus comprising:
one or more force-applying elements, configured to:
reversibly couple to the conduit, and
apply force to the conduit when coupled to the conduit; and
a controller, configured to:
control the force-applying elements such that the force-applying elements apply the force to the conduit, thereby forcing the urine from the conduit in a downstream direction, and
calculate a volume of the urine that was forced, based on the controlling of the force-applying elements.

2. The apparatus according to claim 1, further comprising the conduit.

3. The apparatus according to claim 1, wherein the force-applying elements comprise an actuator configured to reversibly couple to the conduit, and to apply the force to the conduit, via a fluid in a fluid-filled tube.

4. The apparatus according to claim 3, further comprising a pressure sensor configured to:
couple to the fluid-filled tube so as to sense a fluid pressure of the fluid, and
communicate, to the controller, a signal indicating the fluid pressure,
wherein the controller is configured to control the force-applying elements responsively to the signal.

5. The apparatus according to claim 1, wherein the conduit includes a chamber including a moveable wall, and wherein the force-applying elements are configured to apply the force to the moveable wall.

6. The apparatus according to claim 1, further comprising a case coupled to the force-applying elements, wherein the force-applying elements are configured to reversibly couple to the conduit by virtue of the case reversibly coupling to the conduit.

7. The apparatus according to claim 6, wherein the conduit is at least partly contained in a cartridge, wherein the case is shaped to define a slot, and wherein the case is configured to reversibly couple to the conduit via insertion of the cartridge into the slot.

8. The apparatus according to claim 6, wherein the conduit is coupled to one or more latches, and wherein the case is configured to reversibly couple to the conduit by virtue of the latches latching onto the case.

9. The apparatus according to claim 6, wherein the case comprises one or more latches configured to latch onto a housing of the conduit, thereby reversibly coupling the case to the conduit.

10. The apparatus according to claim 6, wherein the case comprises:
an electrical interface connected to the controller, and configured to couple to a cable such that the controller is powered via the cable; and
a communication interface connected to the controller and configured to couple to the cable,
wherein the controller is configured to communicate the calculated volume, or a parameter derived therefrom, via the communication interface and the cable.

11. The apparatus according to claim 1, wherein the force-applying elements comprise:
a pressing element; and
an actuator, configured to apply the force to the conduit by causing the pressing element to press against the conduit,
wherein the controller is configured to control the actuator.

12. The apparatus according to claim 11, wherein the conduit includes a tube, and wherein the pressing element is configured to press against the tube.

13. The apparatus according to claim 12, wherein the pressing element comprises a rotor configured to rotate while pressing against the tube.

14. The apparatus according to claim 11, wherein the actuator is further configured to measure a reciprocal force exerted by the conduit on the pressing element, and wherein the controller is configured to control the actuator responsively to the reciprocal force.

15. The apparatus according to claim 11, wherein the actuator comprises an encoder configured to detect a position of the pressing element, and wherein the controller is configured to control the actuator responsively to the position.

16. The apparatus according to claim 1, further comprising a sensor configured to communicate, to the controller, a signal that varies as a function of an amount of the urine in the conduit or in the bladder, wherein the controller is configured to control the force-applying elements responsively to the signal.

17. The apparatus according to claim 16, wherein the sensor comprises a pressure sensor configured to couple to the conduit so as to sense a pressure in the conduit, and wherein the signal indicates the pressure.

18. The apparatus according to claim 16,
wherein the conduit includes an expandable portion configured to expand as the urine flows into the expandable portion,
wherein the sensor is configured to sense a degree of expansion of the expandable portion, and
wherein the signal indicates the degree of expansion.

19. The apparatus according to claim 18, wherein the expandable portion includes a reservoir disposed upstream from a portion of the conduit to which the force is applied.

20. The apparatus according to claim 18, wherein the expandable portion includes a moveable wall and is configured to expand via movement of the moveable wall, and wherein the force-applying elements are configured to apply the force to the moveable wall.

21. The apparatus according to claim 18, wherein the sensor comprises a pressure sensor configured to sense a pressure that varies with the degree of expansion.

22. The apparatus according to claim 18, wherein the sensor comprises an optical sensor configured to sense the degree of expansion by emitting light at the expandable portion.

23. The apparatus according to claim 16, wherein the conduit is coupled to a first electrical interface configured to connect to the sensor, and wherein the force-applying elements are coupled to a second electrical interface connected to the controller and configured to contact the first electrical interface, when the force-applying elements are coupled to the conduit, such that the sensor communicates the signal to the controller via the first electrical interface and second electrical interface.

24. The apparatus according to claim 16, further comprising a pressure-conveying tube configured to couple to the conduit and to contain a fluid such that a fluid pressure of the fluid varies with an internal pressure in the conduit,
wherein the sensor comprises a pressure sensor configured to couple to the pressure-conveying tube so as to sense the fluid pressure, and
wherein the signal indicates the fluid pressure.

25. The apparatus according to claim 1, further comprising an optical sensor configured to sense a visual parameter of the urine and to communicate, to the controller, a signal indicating the visual parameter.

26. A method for use with a conduit that is configured to carry urine downstream from a bladder of a subject, the method comprising:
controlling one or more force-applying elements, which are reversibly coupled to the conduit, such that the force-applying elements apply force to the conduit, thereby forcing the urine from the conduit in a downstream direction; and
calculating a volume of the urine that was forced, based on the controlling of the force-applying elements.

27. The method according to claim 26, wherein controlling the force-applying elements comprises controlling an actuator such that the actuator applies the force to the conduit via a fluid in a fluid-filled tube.

28. The method according to claim 27, wherein controlling the actuator comprises controlling the actuator in response to a signal indicating a fluid pressure of the fluid.

29. The method according to claim 26, wherein controlling the force-applying elements comprises controlling an actuator such that the actuator causes a pressing element to press against the conduit.

30. The method according to claim 29, wherein controlling the actuator comprises controlling the actuator responsively to a reciprocal force exerted by the conduit on the pressing element.

31. The method according to claim 29, wherein the actuator includes an encoder configured to detect a position of the pressing element, and wherein controlling the actuator comprises controlling the actuator responsively to the position.

32. The method according to claim 26, wherein controlling the force-applying elements comprises controlling the force-applying elements responsively to a signal that varies as a function of an amount of the urine in the conduit or in the bladder.

33. The method according to claim 32, wherein the signal indicates a pressure in the conduit.

34. The method according to claim 32,
wherein the conduit includes an expandable portion configured to expand as the urine flows into the expandable portion, and
wherein the signal indicates a degree of expansion of the expandable portion.

35. The method according to claim 32, wherein the signal indicates a fluid pressure of a fluid in a pressure-conveying tube coupled to the conduit such that the fluid pressure varies with an internal pressure in the conduit.

36. Apparatus for use with one or more force-applying elements, the apparatus comprising:
at least one tube, configured to carry urine that flows downstream from a bladder of a subject via a urinary catheter that catheterizes the subject; and
a conduit section configured to couple to the tube in fluid communication with the tube and to reversibly couple to the force-applying elements so as to facilitate the force-applying elements applying force to the conduit section, thereby forcing the urine from the conduit section in a downstream direction.

37-39. (canceled)

40. The apparatus according to claim 36, wherein the force-applying elements are coupled to a case, and wherein the conduit section is configured to reversibly couple to the force-applying elements by reversibly coupling to the case.

41. The apparatus according to claim 40, further comprising a cartridge containing the conduit section.

42. The apparatus according to claim 41,
wherein the case is shaped to define a slot, and
wherein the conduit section is configured to reversibly couple to the case via insertion of the cartridge into the slot.

43. The apparatus according to claim 40, further comprising one or more latches coupled to the conduit section and configured to latch onto the case, thereby reversibly coupling the conduit section to the case.

44. The apparatus according to claim 40, further comprising a housing that houses the conduit section,
wherein the case includes one or more latches configured to latch onto the housing, and
wherein the conduit section is configured to reversibly couple to the case by virtue of the latches latching onto the housing.

45. The apparatus according to claim 36, wherein the force-applying elements include a pressing element configured to apply the force to the conduit section by pressing against the conduit section.

46. The apparatus according to claim 45, wherein the conduit section comprises a peristaltic pump tube, and wherein the conduit section is configured to reversibly couple to the pressing element so as to facilitate the pressing element pressing against the peristaltic pump tube.

47. The apparatus according to claim 36, wherein the conduit section comprises a chamber comprising a moveable wall, and wherein the conduit section is configured to reversibly couple to the force-applying elements so as to facilitate the force-applying elements applying the force to the moveable wall.

48. The apparatus according to claim 36, further comprising a reservoir disposed upstream from the conduit section.

49. The apparatus according to claim 48, wherein the reservoir is configured to expand as the urine flows into the reservoir.

50. The apparatus according to claim 48, further comprising a sensor configured to communicate a signal that varies as a function of an amount of the urine in the reservoir.

51. The apparatus according to claim 36, further comprising a sensor configured to communicate a signal that varies as a function of an amount of the urine in the conduit section.

52. The apparatus according to claim 36, further comprising a pressure sensor configured to sense an outlet pressure at an outlet of the urinary catheter and to communicate a signal indicating the outlet pressure.

53. The apparatus according to claim 36, further comprising a connection port coupled to the tube or to the conduit section and configured to couple to a pressure sensor such that the pressure sensor senses an internal pressure in the tube or in the conduit section.

54. The apparatus according to claim 36, further comprising a connection port coupled to the tube or to the conduit section and configured to couple to a pressure-conveying tube containing a fluid such that a fluid pressure in the pressure-conveying tube varies in response to an internal pressure in the tube or in the conduit section.

55. The apparatus according to claim 54, further comprising the pressure-conveying tube.

56. The apparatus according to claim 36, further comprising a first electrical interface coupled to the conduit and configured to connect to a sensor, wherein the force-applying elements are coupled to a second electrical interface connected to a controller and configured to contact the first electrical interface, when the conduit section is coupled to the force-applying elements, such that the sensor communicates a signal to the controller via the first electrical interface and second electrical interface.

57-171. (canceled)
